(19) United States
(12) Patent Application Publication
(10) Pub. No.: US 2007/0122852 A1
(43) Pub. Date: May 31, 2007

(54) Method for identifying antigen specific b cells

(75) Inventors: Patrick Bauerle, Gauting (DE); Patrick Hoffman, Penzberg (DE); Susanne Weinberger, Munchen (DE); Roman Kischel, Zorneding (DE)

Correspondence Address: FULBRIGHT & JAWORSKI L.L.P., 600 CONGRESS AVE., SUITE 2400, AUSTIN, TX, 78701, US

(73) Assignee: Micromet AG

(21) Appl. No.: 10/534,788

(22) PCT Filed: Nov. 12, 2003
(86) PCT No.: PCT/EP03/12664

§ 371 Date: Apr. 12, 2006

(30) Foreign Application Priority Data
Nov. 13, 2002 (EP) 02025335.7

(51) Int. Cl. C40B 30/06; C40B 50/06; C40B 40/10; G01N 33/567; C07K 16/28

(52) U.S. Cl. 435/7.21; 435/70.21

(57) ABSTRACT

The present invention relates to a method of identifying a B cell carrying a surface immunoglobulin molecule having a binding site for an antigen of interest comprising contacting a sample putatively containing said B cell with the antigen of interest wherein said antigen is labeled with a first label and with a receptor specifically binding to said surface immunoglobulin molecule wherein said receptor is labeled with a second label and wherein said first label, when being brought into a spatial proximity of between 10 and 100 Angstrom with said second label emits a detectable signal upon activation of said second label by an external source and assessing the presence of said detectable signal, wherein said presence is, in turn, indicative of the B cell carrying a surface molecule having a binding site for the antigen of interest.

The present invention relates to a method of identifying a B cell carrying a surface immunoglobulin molecule having a binding site for an antigen of interest comprising contacting a sample putatively containing said B cell with the antigen of interest wherein said antigen is labeled with a first label and with a receptor specifically binding to said surface immunoglobulin molecule wherein said receptor is labeled with a second label and wherein said first label, when being brought into a spatial proximity of between 10 and 100 Angstrom with said second label emits a detectable signal upon activation of said second label by an external source and assessing the presence of said detectable signal, wherein said presence is, in turn, indicative of the B cell carrying a surface molecule having a binding site for the antigen of interest.

In this specification, a number of documents is cited. The disclosure content of these documents including manufacturers' manuals, is herewith incorporated by reference in its entirety.

There is a high medical interest in therapeutic applications of antibodies in human patients. Monoclonal antibodies are routinely produced according to established procedures by hybridomas generated by fusion of mouse lymphoid cells with an appropriate mouse myeloma cell line (first published by Köhler & Milstein, 1975, Nature 256, 495). Therapeutical administration of murine monoclonal antibodies, however, may have severe side effects. For example, in patients with minimal residual colorectal cancer, a murine monoclonal antibody specific for the human 17-1A-antigen decreased the 5-year mortality rate by 30% compared to untreated patients; in total each patient was treated with 900 mg of murine antibody (Riethmüller, Lancet 343(1994), 1177-1183). However, during the course of antibody treatment patients developed a strong antibody response against murine immunoglobulin.

Mouse antibodies are per definition 100% mouse-derived and are recognized as foreign bodies by the human immune system, resulting in an immune response against the drug, specifically a human anti mouse antibody (HAMA) response. As a result, the antibody drug is neutralised on repeated dosing. This results in rapid clearance of the drug from the body and possible allergic responses. Moreover, preformed HAMAs induced by former antibody treatment or another contact with murine immunoglobulin can severely interfere with later antibody therapies. Therefore, drugs based on murine antibodies can only be used in acute indications, where the patient is treated once or at most twice.

Due to those problems associated to murine antibodies, it has been a challenge to develop methods for the production of antibodies useful for antibody therapy which do not have the disadvantage of producing HAMA.

In one approach, chimaeric antibodies were developed (Boss, 1989, U.S. Pat. No. 4,816,397; Cabilly, 1989, U.S. Pat. No. 4,816,567). Chimaeric antibodies are composed of human and non-human amino acid sequences. Such chimaeric antibodies are genetically engineered. They contain approximately 66% human and 33% non-human protein. Accordingly, hybrid antibody molecules have been proposed which consist of amino acid sequences from different mammalian sources. The chimaeric antibodies designed thus far comprise variable regions from one mammalian source, and constant regions from human or another mammalian source (Morrison et al. (1984) Proc. Natl. Acad. Sci. USA., 81:5851-6855; Neuberger et al. (1984) Nature 312: 604-608; Sahagan et al. (1986) J. Immunol. 137:1066-1074; European patent applications EP 04302368.0 (Genentech); EP 85102665.3 (Research Development Corporation of Japan); EP 85305604.2 (Standord); PCT application PCT/GB85/00392 (Celltech Limited). Chimaeric antibodies potentially have improved therapeutic value as they presumably elicit less circulating human antibody against the non-human immunoglobulin sequences. However, an immune response, the so-called human anti-chimaera antibody (HACA) response, is often generated against such drugs.

Therefore, humanised monoclonal antibodies have been designed (Adair, 1999, U.S. Pat. No. 5,859,205; Queen, 1996, U.S. Pat. No. 5,530,101). Humanised antibodies differ from chimaeric antibodies in that they contain close to 90% human-derived protein sequence, including a largely human-derived variable domain sequence. This is made possible by retaining the minimum non-human sequence required to retain the original monoclonal antibody's binding properties. The variable domain of humanised antibodies usually consists of a human antibody framework (FR) and the complementary determining regions (CDRs) of the parental (murine) antibody, which provides the binding specificity. Humanised antibodies, however, tend to have reduced substrate-binding activity and may still provoke an immune response. (Dr. Sydney Welt, May 1998, Cancer Research Institute (CRI) Symposium, New York, “The use of humanized antibodies to treat cancer”). Generally, to prevent the problems of HAMA and HACA, therapeutic antibodies with minimal immunogenicity but which still possess high substrate binding activity, would be preferable.

To achieve this goal, it has, of course, been envisaged to use therapeutic antibodies or antibody derivatives that are completely human by their amino acid sequence and wherein the immunogenic profile of the human antibody idiotype is minimized by using human Ig-variable regions likely to be tolerated by the human immune system.

Several techniques have been developed to generate human antibodies.

1.) Human hybridoma or other human cell immortalisation methods have been developed but proved to be quite inefficient in generating human antibody producing cell lines compared to the murine hybridoma technology. Human monoclonal antibodies are difficult to produce by cell fusion techniques since, among other problems, human hybridomas are notably unstable, and removal of immunized spleen cells from humans is not feasible. It has proven difficult to find suitable human myeloma-fusion partners. Human-human hybrids are not as stable and do not produce as great a quantity of antibody as can be attained in mouse-mouse fusion systems. With the application of in vitro immunisation using human cells, another difficulty is that human cells contain various repressed lethal viruses which may be activated and expressed upon hybridisation and subsequent recombination. These viruses can be infectious, and pose issues of health and safety for lab workers. Furthermore, it is difficult to totally remove all lethal viruses from the monoclonal antibodies, and thus such antibodies cannot readily be used therapeutically for humans. Another difficulty of the hybridoma technology lies in the fact that naturally rarely occurring antibodies and corresponding B lymphocytes are rarely immortalized. Namely, the size of the original pool of hybridomas is limited by the number of stable antibody clones that can be generated and screened in a reasonable time and by the intrinsic inefficiency of the process. Thus, of the antibody producing cells present in the population of immunized cells that are subjected to the fusion process, only a small fraction form stable antibody-producing hybrids and are available to a screen for the desired antibody. Furthermore, antibodies must be subcloned in a tedious growth and subcloning process during which the desired antibody-forming cell may be lost. If the desired antibody is formed by only a small fraction of antibody-forming cells involved in an immune response and is, for example, an antibody which mimics an enzyme or an autoreactive antibody, the likelihood that this antibody will be produced by any of the stable hybrids available for screening is correspondingly small.

2.) Human antibodies have become much more readily accessible since the availability of transgenic mice expressing human antibodies (Brüggemann, Immunol. Today 17 (1996), 391-397). The transgenic technology involves the introduction of human antibody genes info the mouse genome. Advantages of transgenic technologies include fully human protein sequences, high affinity, and fast and efficient production processes. However, a potential drawback of the technique is that it is difficult to introduce enough of the human antibody genes to ensure that the mice are capable of recognising the broad diversity of antigens relevant for human therapies. In addition, transgenic animals are very difficult to generate and antibodies with certain specificities even more laborious to find.

3.) Another way for human antibody production is the combinatorial antibody library and phage display technology allowing the in vitro combination of variable regions of Ig-heavy and light chains. (VH and VL) and the in vitro selection of their antigen binding specificity (Winter, Annu. Rev. Immunol. 12 (1994), 433.455). By using the phage display method, rare events like one specific binding entity out of 10⁷ to 10⁹ different VL/VH- or VH/VL-pairs may be isolated; this is especially true when the repertoire of variable regions has been enriched for specific binding entities by using B-lymphocytes from immunized hosts as a source for repertoire cloning. With combinatorial phage libraries, the problem occurs that often the frequency of specific binding entities is substantially lowered in naturally occurring antibody repertoires. This is particularly true for cases of antibodies binding to self-antigens. Random combinations of VL- and VH-regions from a self-tolerant host resulting in combinatorial antibody library of a conventional size (10⁷ to 10⁹ independent clones) most often are not sufficient for the successful in vitro selection of rare antibody specificities by the phage display method. To isolate low frequency antigen-specificities it is possible to use very large combinatorial antibody libraries that compensate by the library size for the low frequency of autoreactive antibodies in naturally occurring repertoires. Combinatorial antibody libraries exceeding a size of 10⁹ independent clones, however, are difficult to obtain because of the current technical limit of the transformation efficiency for plasmid-DNA into E. coli-cells.

To avoid the self-tolerance mediated bias in naturally occurring antibody repertoires, that underrepresents autoreactive antibodies and markedly decreases the chances of isolating antibodies specifically recognizing self-antigens, approaches using semisynthetic or fully synthetic VH- and/or VL-chain repertoires have been developed. For example, almost the complete repertoire of unrearranged human V-segments has been cloned from genomic DNA and used for in vitro recombination for functional variable region genes, resembling V-J or V-D-J-recombination in vivo (Hoogenboom, J. Mol. Biol. 227 (1992), 381-388; Nissim, EMBO J. 13 (1994) 692-698; Griffiths, EMBO J. 13 (1994), 3245-3260). Usually, the V-D-/D-J-junctional and the D-segment diversity mainly responsible for the extraordinary length and sequence variability of heavy chain CDR3 as well as the V-J-junctional diversity contributing to the sequence variability of light chain CDR3 is imitated by random sequences using degenerated oligonucleotides in fully synthetic and semisynthetic approaches (Hoogenboom (1994), supra; Nissim, supra; Griffiths, supra; Barbas, Proc. Natl. Acad. Sci. U.S.A 89 (1992), 4457-4461).

Synthetic human libraries often have the disadvantage that they are difficult and laborious to create and screening for a certain specificity needs high throughput tools. Further, VL/VH- or VH/VL-pairs selected for binding to a human antigen from such systematic repertoires based on human V-gene sequences are at risk of forming immunogenic epitopes that may induce an undesired immune response in humans (Hoogenboom, TIBTECH 15 (1997), 62-70). Especially the CDR3-regions derived from completely randomised sequence repertoires are predestined to form potentially immunogenic epitopes as they have never had to stand the human immune surveillance without being recognized as a foreign antigen resulting in subsequent elimination. This is equally true for human antibodies from transgenic mice expressing human antibodies as these immunoglobulin molecules have been selected for being tolerated by the murine but not the human immune system.

Quite often the success of any one of these methods largely depends on the frequency with which the desired antigen specificity is represented in the source material. Antibodies with an antigen specificity directed against an antigen that the individual was previously immunized with, will constitute a high percentage of the total reservoir of antigen-specificities present in the pool. Antibodies of naïve, unprimed B cells, where no previous immunization has taken place, will be represented to a much lower percentage in the total reservoir of antigen-specificities present in the pool. The most rarely occurring antibodies are those that have undergone a previous counter-selection like the antigen-specificities of autoreactive antibodies. Furthermore, antibodies directed against self red blood cells are also part of antibodies occurring with very low frequency. The chances of isolating an antibody with antigen-specificity against an auto-antigen or against a self red blood cell by the methods described above are extremely low.

Prior art approaches to isolate low-frequency antibody specificities include those described in U.S. Pat. No. 5,326,696 and in U.S. Pat. No. 5,627,052. U.S. Pat. No. 5,326,696 assigned to Tanox Biosystems, Inc., describes a method for identifying and isolating low-frequency B-cells that relies on the use of two antigen populations wherein the antigen populations differ by their fluorescent labels. B-cells carrying Ig molecules with the desired specificity for the antigen on their surface will bind to the labeled antigens. Using a multi-channel FACS machine, those B-cells are isolated that have picked up both type of antigens, i.e. antigens labeled with the first and with the second fluorescent label. The fidelity of the method may be enhanced by counter selecting against autofluorescent cells and sticky cells of various leukocyte subpopulations as well as by additionally marking B-cells with a labeled receptor for B-cell specific surface antigens such as CD19, y-chain, κ or λ-chain, or Fc-receptors. In the case that the additional selection means are employed, fluorescent labels different from the labels attached to the desired antigens are necessary. Thus, the claimed invention envisages four different labels for an optimal selection and a correspondingly equipped FACS machine. It is of note that the optional additional labeling of surface components of B-cells cannot enhance to any large extend the frequency of B-cells expressing antibody to the desired antigen but is useful for clearing the non-specific contamination of T-cells, macrophages, monocytes, B-cells expressing IgM and other cells.

U.S. Pat. No. 5,627,052 assigned to B. R. Centre, Ltd., describes a process for the identification of a protein of choice, preferably of an antibody with a desired specificity from which the variable regions may be cloned and subsequently employed to generate a novel protein of interest. The claimed invention makes use of a functional assay for identifying the antibody of interest. The functional assay relies on the suspension of antibody-forming cells in a medium wherein the medium comprises an indicator system which indicates the presence and location of the antibody forming cells. The indicator medium may contain, for example pathogenic microorganisms and cells susceptible in viability to said pathogenic microorganisms. If the sample to be accessed comprises an antibody with specificity to the pathogenic microorganism, it will inhibit infection of the susceptible cells by the pathogenic microorganism. As a consequence and surrounding the cell capable of producing the desired antibody, a layer of cells susceptible to the pathogenic microorganism will grow due to the inhibition of the pathogenic effects normally exerted by the microorganism due to the presence of the antibody. Cells producing the desired antibody may then be subjected to conventional recombinant DNA technologies and V_{H} and V_{L} region genes involved in the antibody production may be cloned. Alternatively, the selection system makes use of, for example, haemolytic plaques assays involving coupling the antigen to the erythrocyte surface, rosetting techniques or techniques relying on the enhanced growth or morphological change of cells due to the presence of antibodies having an effect analogous to a protein selected from a group of differentiation and growth factors. The claimed method is allegedly suitable to detect antibody forming cells even if present in a very low frequency in a sample only. However, the selection step is time consuming and only useful for the analysis of a confined number of antibody-producing cells.

As discussed, the methods described above for the generation of human or humanized antibodies are not suitable for the convenient and reliable isolation of very rare antibodies, in particular non-immunogenic autoreactive antibody specificities. On top of this, the prior art did not even disclose methods to reliably identify and isolate corresponding rare B cells. The technical problem underlying the present invention therefore was to provide such methods. Starting from rarely naturally occurring B cells, antibody genes giving rise to the desired antibody specificities might then be cloned and used for the desired downstream developments.

The solution to said technical problem is achieved by providing the embodiments characterized in the claims.

Accordingly, the present invention relates to a method of identifying a B cell carrying a surface immunoglobulin molecule having a binding site for an antigen of interest comprising (a) contacting a sample putatively containing said B cell (aa) with the antigen of interest wherein said antigen is labeled with a first label and (ab) with a receptor specifically binding to said surface immunoglobulin molecule wherein said receptor is labeled with a second label and wherein said first label, when being brought into a spatial proximity of between 10 and 100 Angstrom with said second label emits a detectable signal upon activation of said second label by an external source and (b) assessing the presence of said detectable signal, wherein said presence is, in turn, indicative of the B cell carrying a surface molecule having a binding site for the antigen of interest.

The term “surface immunoglobulin molecule” refers to immunoglobulin molecules inserted by way of their C-terminus into the surface of B cells. In principle, this term is well established in the art; see, for example, W. E. Paul (ed.) “Fundamental Immunology”, second edition 1989, Raven Press, New York, Roitt et al, “Immunology”, 1985, The C. V. Mosby Company, St. Louis, Mo. It includes sIgM, sIgD, sIgA, sIgG and sIgE and all subclasses thereof. In the following, these surface immunoglobulins are also referred to as IgM, IgD, IgA, IgG and IgE.

The term “receptor” refers to a molecule that is capable of specifally recognizing and binding to an epitope of the surface immunoglobulin molecule. Potential receptors include aptamers and antibodies.

The term “activation” according to the present invention describes a transient or perpetual change in the energy level of the respective molecules. Advantageously, “activation” means an excitation generated e.g. by a laser source. In another preferred interpretation, “activation” relates to a substrate turnover, such as coelenterazine, which is a substrate for the enzyme luciferase (Wang, 2002, Mol. Genet. Genomics 268 (2), 160-168).

A “detectable signal” means, in accordance with the present invention, any signal that can be qualitatively or quantitatively assessed by means of a suitable signal detector. Such signals include phosphorescent, bioluminescent and fluorescent signals.

The term “B-cell” in the present invention comprises all lymphocytes that develop in the adult bone marrow or in the fetal liver and are destined to produce antibodies. All different stages in the development of a B cell are included, such as pre B cells, naïve, unprimed B-cells, which have not come into contact with an antigen yet or mature B cells, as well as plasma cells, which have been activated to proliferate and mature through antigen contact.

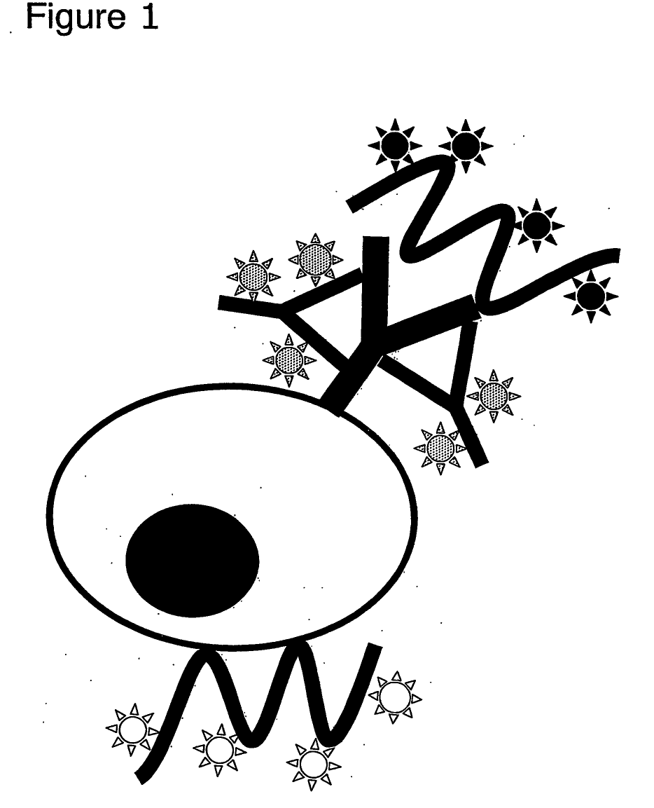

As has been outlined above and in other terms, the invention solves the recited technical problem by a highly sensitive positive selection approach. B cells are isolated from a sample e.g. peripheral blood mononuclear cells (PBMCs) from the blood stream and labeled using two different detectable labels such as fluorescent dyes. One label is coupled to the antigen of choice for which a corresponding antigen-specificity shall be found. The second label is coupled to a receptor such as a monoclonal or polyclonal (serum-derived) antibody specific for the surface immunoglobulin molecule. The surface immunoglobulin may be an IgD surface marker on naïve unprimed B cells (FIG. 1 schematic). Most cells from the cellular sample will not bind the antigen. Only very few antigen-specific B cells will recognize the antigen. In those cases, in our example the IgD-coupled label (such as a fluorochrome) and the antigen coupled label (such as second fluorochrome) come into close spatial proximity. In case of antibody coupled labels, such antibodies may be monoclonal or polyclonal antibodies, which may be directed against any epitope on the surface receptor which is not the antigen binding epitope or an epitope too distant from the antigen binding epitope in order to allow fluorescence resonance energy transfer between the two labels to occur. If the fluorochromes chosen constitute a donor-acceptor pair, then there exists a FRET system. The selection of antigen-specificities is highly sensitive and specific due to the discriminative power of the Förster distance. Therefore, even the isolation of extremely rare antigen-specificities like autoreactive antigen-specificities is possible. This is preferably accomplished by the use of a fluorescence activated cell sorter (FACS) in combination with fluorescence resonance energy transfer (FRET) as a selection principle. The principles of FACS-based analysis as well as of the FRET principle are well established in the art and briefly outlined herein below.

FACS (fluorescence activated cell sorter) denominates a cytofluorimetric device that allows the analysis and isolation of cell populations according to the scattering and the fluorescent signals of those cells. Therefore, the cells get labeled with fluorescent dyes which are usually coupled to antibodies that recognize a certain cell type (Römpp Lexikon, 1999, Biotechnologie und Gentechnik, Georg Thieme Verlag, 2^{nd} edition). The resulting signals are detected using e.g. a photo multiplier, CCD- and CMOS-detectors, and photon counting assemblies.

Fluorescence energy transfer (FRET) is a process by which a fluorophore donor in an excited state may transfer its excitation energy to a neighbouring chromophore acceptor non-radioactively through dipole-dipole interactions. In principle, if one has a donor molecule whose fluorescence emission spectrum overlaps the absorbance spectrum of a fluorescent acceptor molecule, they can exchange energy between one another through a non-radioactive dipole-dipole interaction. This energy transfer manifests itself by both quenching of donor fluorescence in the presence of acceptor and increased emission of acceptor fluorescence. Energy transfer efficiency varies most importantly as the inverse of the sixth power of the distance separating the donor and acceptor chromophores. The critical distance is the so-called Föster distance (usually between 10-100 Angstrom). The phenomenon can be detected by exciting the labeled specimen with light of a wavelength corresponding to the maximal absorption (excitation) of the donor and detecting light emitted at the wavelengths corresponding to the maximal emission of the acceptor, or by measuring the fluorescent lifetime of the donor in the presence and absence of the acceptor. The dependence of the energy transfer efficiency on the donor-acceptor separation provides the basis for the utility of this phenomenon in the study of cell component interactions. The conditions that need to exist for FRET to occur are: (1) the donor must be fluorescent and of sufficiently long lifetime; (2) the transfer does not involve the actual reabsorption of light by the acceptor; and (3) the distance between the donor and acceptor chromophores needs to be relatively close (usually within 10-50 Angstrom) (Herman, 1998, Fluorescence Microscopy, Bios scientific publishers, Springer, 2^{nd} edition, page 12)

A further possibility to generate a signal is given with the so called “bioluminescence energy transfer” (BRET) system. This system is described in Arai et al., 2001, Anal. Biochem. 289 (1), 77-81. Said BRET system can also be used for the present invention and its sensitivity can be even higher than that of FRET. The example given in Arai et al. comprises Renilla luciferase, (Rluc) and enhanced yellow fluorescent protein (EYFP).

Further, intramolecular energy transfer has been shown between Renilla luciferase (Rluc) and Aequorea “green fluorescent protein” (GFP) (Wang et al. 2002, Mol. Genet. Genomics 268(2), 160-8). In the presence of the luciferase substrate coelenterazine a GFP emission could be measured at the wave length of 508 nm, without UV excitation. Thus a “double emission” at 475 nm (luciferase) and 508 nm (GFP) could be measured.

Furthermore, donor acceptor interactions in the systematically modified lanthanides such as Ru(II)-Os(II) have been described (Hurley & Tor, 2002, J. Am. Chem. Soc. 124(44), 1323-13241). Analyzes showed a Förster dipole-dipole energy transfer mechanism.

The present invention, in contrast to U.S. Pat. No. 5,326,696, relies on only one detectable signal and thus significantly simplifies the handling of the experiments as well as the necessary technical equipment of the FACS machine employed. In addition, the method of the present invention bears advantages over the prior art multicolor approach because multicolor staining can easily cause false positive results due to unspecific staining. For example, if phycoerythrin (PE) is used as fluorochrome it can, due to its size, cause quenching of the fluorescein signal. As a consequence, the multicolor staining signal can be lost. This is also shown in Reference example 1, where a multicolor sort system was used in order to isolate B cells specific for a defined antigen. With this multicolor system it was however not possible to reliably sort the B cells specific for said antigen. The FRET signal generated by the method of the present invention, however, only occurs if both probes (antigen and anti-surface immunoglobulin) have bound very closely together (Förster distance). Additionally, the fluorochromes used in multicolor FACS selection partially overlap, especially Texas red and allophycocyanin (APC). Therefore, it is problematic to apply multicolor FACS as a selection principle to very rare cells. The extreme gating, which is necessary in this case, results in quenching of signals. Accordingly, cells which actually fulfil the selection criteria, are expected to be lost. Further, without the use of a propidium iodide counter staining to eliminate dead cells the multicolor FACS assay becomes even more difficult to handle and the recovery of living antigen-specific auto reactive B cells is expected to be extremely poor. Recovery of living cells is important, however, if subsequent efficient RNA recovery and V region cloning are envisaged.

Another principal problem with the multicolor FACS selection method is unspecific binding. Antigenic peptides are prone to stick unspecifically to cell surfaces or bind unspecifically to other surface proteins like CD45. Even with the additional signal from anti IgG antibody conjugate or anti CD19 antibody conjugate as suggested in U.S. Pat. No. 5,326,696 the false positive signal remains. In the method of the invention, in contrast, unspecific signals are eliminated. The signal only occurs when antigen and anti-surface immunoglobulin have bound very close together such that FRET occurs (Föster distance). This results in

-a significantly increased specificity.

In accordance with the present invention and according to the example fluorochromes Fluorescein and Alexa Fluor 546, it is preferred that the spatial proximity amounts to at least 50 Angstrom.

The mammalian immune systems such as the human immune system selects against immune competent cells and molecules that are specific for self-antigens. Dysregulation of the immune system in this regard may result in autoimmune diseases such as rheumatoid arthritis or allergy. However, therapeutically it would be advantageous to have autoreactive antibodies that are directed to antigens expressed in the mammalian, and in particular, the human body. Such antigens are, for example, tumor associated antigens. B cells producing such autoreactive antibodies are relatively efficiently depleted from naturally occurring antibody repertoires due to the mechanisms mediating self-tolerance. 90% of the B cells produced every day die without ever leaving the bone marrow (Kuby, 2000, Immunology, 4^{th} edition, W.H. Freeman and company, page 273). Some of this loss is attributed to negative selection and clonal deletion. However, some of those autoreactive B cells escape the clonal deletion process and enter the peripheral human blood stream. This reservoir of natural fully human antibodies potentially represents a broad diversity of antigen specificities and can serve as a valuable source for the isolation of fully human antibody sequences useful in therapeutic applications. Due to the negative selection process in the bone marrow, however, such fully human autoreactive antibody specificities are extremely rare in the total reservoir of antibody specificities. Clonal deletion operates early in life (Burnet, 1959, The clonal selection theory of acquired immunity, Cambridge University Press, London). Later in life all autoreactive B cells not eliminated during ontogeny are prevented from expanding and secreting anti-self antibodies by a compensatory suppressor mechanism (Cunningham A. J., 1976, Transplant. Rev. 31, 23). Therefore, autoreactive antibodies are produced only in minute quantities allowed by the suppressor mechanism (Tomer & Schoenfeld, 1988, Immunological Investigations 17(5), 389-424). It is thus extremely rare to find a certain antigen-specificity against auto-antigens within the population of mature naïve unprimed B cells. Further, primed B cells, which are also present in peripheral blood, are over represented in their antigen-specificity due to clonal proliferation. Accordingly, the probability of finding such antibody specificities in the peripheral blood stream is very low.

Consequently, in a preferred embodiment of the method of the present invention said B cell is an autoreactive B cell.

In another preferred embodiment of the method of the present invention said surface immunoglobulin molecule is an IgD, an IgE, an IgM or an IgG. During the antigen-independent phase of B cell development the B cells mature in the bone marrow. Once the B cells express membrane-bound IgM and IgD immunoglobulins they are mature and leave the bone marrow. Subsequently, when those naïve B cells encounter an antigen the cells are activated and switch their immunoglobulin production to other classes like IgG (Kuby, 2000, Immunology, 4^{th} edition, W.H. Freeman and company, page 269). Therefore, membrane-bound IgD is a marker molecule for the naïve unprimed B cell population. This is the population which comprises rare autoreactive antibody producing B cells.

Consequently, in a particularly preferred embodiment said B-cell is a naïve, IgD-positive B-cell.

The method of the invention is suitable, in principle, to identify B cells carrying surface receptors against abundantly occurring or rarely occurring antigens. The specific advantages of the method of the invention in particular take effect when it comes to the isolation of rarely occurring antigens as has been outlined above. Such rarely occurring antigens may belong to the group of receptors and cellular proteins or fragments thereof. In a preferred embodiment of the method of the present invention said antigen of interest is selected from the group consisting of auto-antigens, allergens and immunoglobulins.

The term “auto-antigen” means, in accordance with the present invention, any self antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

The problems with the isolation of antibodies against auto-antigens have been detailed herein above. Similarly, antibodies to allergens, in particular belonging to the IgE class, have rarely been identified by conventional technologies. Inter alia, this is due to the low frequency of 0.3 μg/ml IgE antibodies in the serum (Kuby, 2000, Immunology, 4^{th} edition, W.H. Freeman and company, page 101). Rheumatoid factors rarely have been isolated as well. Rheumatoid factors are a dominant class of autoantibodies in rheumatoid arthritis and certain other autoimmune syndromes. They are IgM or IgG antibodies formed against IgG immunoglobulins, which is usually triggered by slight alterations of such IgGs. High affinity rheumatoid factor B cells are essentially lacking in high affinity rheumatoid factor transgenic mice. Analysis of bone marrow suggests that central tolerance prevents high affinity rheumatoid factor B cell development, receptor editing, or both (Wang & Shlomchik, 1997, J. Immunol. 159, 1125-1134).

The formation of rare autoreactive antibodies may also be triggered by environmental factors such as the sun, drugs or infections (Abu-Shakra & Schoenfeld, 1991, Immunol. Ser. 55, 285-313). Such autoantibodies may belong to different immunoglobulin classes and include rheumatoid factor, anti-DNA, anticardiolipin, and anti-red blood cell antibodies. The association between infectious agents and autoimmune disorders was reported with acute infections as well as with infections with a chronic course. The appearance of rheumatic fever was observed following streptococcal infection (Zabriskie, 1982, Pediatr. Ann. 11, 383-396) and the onset of diabetes mellitus type I following mumps or coxsackievirus infection (King et al., 1983, Lancet 1, 1397-1399; Christiansen et al., 1983, Sem. Arthritis Rheum. 17, 1-23). Also, an association between autoimmunity and tuberculosis, syphilis, AIDS, malaria, Leishmania, schistosomias, and mycobacterial infection was reported. Mycobacterial infections for example induce autoimmunity via antigenic similarity between host antigens and mycobacteria. The antibodies formed against the mycobacteria subsequently cross react with host antigen depending on the patient's genetic background. An example for the frequency of anti-self red blood cell antibodies showed that only 1/104 000 B cells produced anti-self A/B IgM and 1/350 000 B cells made anti-self A/B IgG (Rieben et al., 1992, Eur. J. Immunol. 22, 2713-2717).

Generally, the sample may be any sample putatively containing B cells. For example, the sample may be serum or lymph. In this case, the source of the sample may be any animal, preferably any mammal and most preferably a human. Alternatively, the source may be a spleen, lymph node, bone marrow or other organ that contains B cells or parts thereof. In these cases, it is preferred that the source is a non-human animal. In a further preferred embodiment of the method of the present invention said sample is a sample of essentially purified B cells. This embodiment is particularly useful for lowering the background in the readout system due to the absence of other cells containing surface molecules potentially being a source of cross-reactivities to the antigen or the receptor such as T cells. Essentially purified B cells may be employed according to techniques well established in the art including Ficoll density gradient centrifugation (Ficoll-Paque from Amersham, density 1.077 g/ml, Amersham Biosciences, Buckinghamshire, UK) or use of Milteny Columns (i.e. magnetic depletion of T cells, Milteny B cell isolation kit, Auburn, Calif., USA) and methods described in the appended examples.

In an additional preferred embodiment of the method of the present invention said first label is a fluorophore or fluorochrome. Fluorophores and fluorochromes are fluorescent agents which, as has been detailed above, can efficiently be employed in FACS analyses, advantageously in combination with FRET analyses. In a particularly preferred embodiment of the method of the present invention said fluorophore is Alexa 546. This particularly advantageous fluorophore is employed, in accordance with the present invention, as a FRET acceptor.

In a further preferred embodiment of the method of the present invention said second label is a fluorophore or fluorochrome.

In a particularly preferred embodiment of the method of the present invention said fluorophore is fluorescein, Cy2, or BODIPY_FL™. These most preferred agents serve in accordance with the invention as a FRET donor.

In another preferred embodiment, said second label is fluorescein and said first label is Alexa 546.

In another preferred embodiment of the method of the present invention said spatial proximity is such that fluorescence resonance energy is transferred from the second to the first label. This technology is also referred to FRET as has been explained above.

Furthermore, advantages of FRET comprise that only the second label, the donor, is excited by a specific wavelength, whereas the signal that is assessed derives from the first label, the acceptor. Thus, a signal only occurs when resonance energy transfer takes places. Consequently, only low background noise occurs and high sensitivity and selectivity of the assay can be achieved.

In a more preferred embodiment of the method of the present invention said receptor is an antibody or a fragment or derivative thereof. Fragments of antibodies include F(ab′)₂ and Fv fragments. Derivatives of antibodies are, for example, single-chain Fv constructs, chimeric as well as humanized antibodies; see also, for example, Harlow and Lane, “Antibodies, A Laboratory Manual”, CSH Press 1989, Cold Spring Harbor. Antibodies include monoclonal and polyclonal antibodies, i.e. serum antibodies.

In a most preferred embodiment of the method of the present invention said antibody is directed against the Fc-part of the surface immunoglobulin molecule. Antibodies against the Fc part of surface immunoglobulins can be easily prepared according to standard procedures. Cross reactivity with different Ig classes is tested for, e.g., by assessing the replacement rate of binding to the surface Ig constant region of choice vs, the unwanted constant regions in a turbidimetric assay. Replacement rate of binding to the surface Ig constant region of choice may also be determined by a competitive assay such as an ELISA where the Ig constant region is coated to the wells and competition between differently labeled antibodies or other substances like peptides is measured. The choice of the Fc portion as the receptor target has the advantage that it minimizes the risk of interference of binding of the surface receptor with the desired antigen. The antigen binding epitope of the receptor has to be located at the Fc portion in such a way that the maximal allowable Förster distance of 100 Angstrom can be achieved between the two labels.

In an additional preferred embodiment of the method of the present invention said antibody is an anti-idiotypic antibody, wherein said anti-idiotypic antibody does not interfere with the binding site to the antigen. If this preferred embodiment is selected in the method of the invention, care needs to be taken that the anti-idiotypic antibody, i.e. the antibody directed to the variable region of the surface immunoglobulin, does not interfere with the binding of the surface immunoglobulin with the antigen of choice. Accordingly, an appropriate test must be performed by the practitioner prior to implementing the method of the invention. Such appropriate tests are available in the art; see, for example, tests described in Harlow and Lane, loc. Cit. which may be slightly modified by the person skilled in the art, if desired. Appropriate tests are for example epitope-mapping which uses overlapping peptides and ELISA, dot blots, or PepScan™ membranes for detection. Radioactively labeled or fluorescently or bioluminescently labeled peptides may be used for competitive studies in solution.

In a further preferred embodiment of the method of the present invention said external source is a laser source. Again, the laser source is particularly appropriate for performing the FRET assay. In a further embodiment of the method of the present invention said laser source is an Argon laser 488.

In another preferred embodiment of the method of the present invention said detectable signal is a light emission detected by a photomultiplier.

In order to clone antibody variable regions, it is important beforehand to isolate the B cells which have been identified with the method of the present invention. Hence, in a particularly preferred embodiment of the present invention the method further comprises the step of isolating identified B cells.

The B cells can, for example, be isolated from samples of peripheral blood gained from humans and as described in Example 4 of the present invention.

In a further particularly preferred embodiment, said B cells are “low frequency” B cells. The term “low frequency” as employed in the present invention describes B cells occurring only rarely in the entire pool of B cells of a sample and mammal, respectively. Consequently, in one embodiment, said low frequency B cells occur at a frequency of about 1 low frequency B cell in 10⁵ of all the B cells in the pool, in another more preferred embodiment they occur at a frequency of about 1 in 10⁶, in a more preferred embodiment in a frequency of about 1 in 10⁷, in an even more preferred embodiment in a frequency of about 1 in 10⁸ and in a most preferred embodiment in a frequency of about 1 in 10⁹ B cells.

An example for low frequency B cells has been mentioned above. This type of rarely occurring B cells produces anti-self red blood cell antibodies in humans (Rieben et al., 1992, Eur. J. Immunol. 22, 2713-2717).

As has been stated above, one of the most important goals of the method of the invention is the cloning of antibody variable regions from the identified B cells. These variable regions may subsequently be employed in the construction of proteins such as antibodies or fragments or derivatives thereof and these agents may be used in therapeutic approaches. Accordingly, in a different preferred embodiment, the method of the present invention further comprises the step of cloning VH- and VL-domains from the selected B cells. Preferably, these V-domains (also referred to as V-regions) comprise the complete functionally rearranged VDJ regions. Alternatively parts thereof such as at least one of the complimentarily determining regions may be cloned. For example, RNA or DNA can be isolated from selected single B cells and the VH and VL regions can be cloned via RT-PCR or PCR using specific primers. These V regions then, can be further subcloned. In one of different alternatives, variable regions may also be cloned by generating cDNA libraries of preferably expanded selected B cells and functionally rearranged variable region genes isolated using appropriate probes. Further suitable approaches have been summarized in U.S. Pat. No. 5,326,696. VH and VL regions may be combined according to their natural sequence or in arbitrary combination. The VH/VL regions may be combined by the means of fusion PCR introducing a linker sequence in between. These VH/VL fusions may further be subcloned into various antibody formats and constructs like complete antibodies, antibody fragments, single-chain antibodies or bispecific constructs i.e. constructs with two different binding specificities (Sambrook & Russel: Molecular Cloning: A Laboratory Manual, third edition 2001, Cold Spring Harbor Laboratory Press, Cold Spring Harbor, N.Y.).

In a particularly preferred embodiment of the method of the present invention said cloning comprises isolating RNA from the selected B cell, followed by an RT-PCR and followed by fusing the DNA or fragments thereof into an expression vector.

The vector employed may be a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector used may comprise expression control elements, allowing proper expression of the coding regions in suitable hosts. Such control elements are known to the artisan and may include a promoter, a splice cassette, translation initiation codon, translation and insertion site for introducing an insert into the vector. Preferably, the DNA is operatively linked to said expression control sequences allowing expression in eukaryotic or prokaryotic cells.

Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook (1989), loc. cit., and Ausubel, Current Protocols in Molecular Biology, Green Publishing Associates and Wiley Interscience, N.Y. (1989), (1994). Relevant sequences can be transferred into expression vectors where expression of a particular (poly)peptide/protein is required. Typical expression vectors include pTRE, pCAL-n-EK, pESP-1, pOP13CAT.

The term “control sequence” refers to regulatory DNA sequences which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which is necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

An “expression vector” is a construct that can be used to transform a selected host cell and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotic and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the P_{L}, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). An alternative expression system which could be used to express a cell cycle interacting protein is an insect system. In one such system, Autographa califomica nuclear polyhedrosis virus (AcNPV) is used as a vector to express foreign genes in Spodoptera frugiperda cells or in Trichoplusia larvae. The coding sequence may be cloned into a nonessential region of the virus, such as the polyhedrin gene, and placed under control of the polyhedrin promoter. Successful insertion of said coding sequence will render the polyhedrin gene inactive and produce recombinant virus lacking coat protein coat. The recombinant viruses are then used to infect S. frugiperda cells or Trichoplusia larvae in which the protein is expressed (Smith, J. Virol. 46 (1983), 584; Engelhard, Proc. Nat. Acad. Sci. USA 91 (1994), 3224-3227).

In a further preferred embodiment the method of the present invention is used as alternative to phage display for the gain of antibodies or fragments thereof. Preferably, the method for the production of such binding molecules further comprising the steps of (a) introducing mutations (e.g. as described in Barbas III, 1996, TIBTECH 14, 230; Schier, 1996, J. Mol. Biol. 263, 551; Hawkins, 1992, J. Mol. Biol. 226, 889) in the sequences encoding said VH- and/or VL-domain/s of at least one of said B cells/antigen binders, wherein said mutations result in amino acid substitutions and wherein the number of mutations ranges from one to thirty, preferably from one to fifteen, more preferably from one to ten and most preferred from one to five, such that one or more modified antibodies is/are obtained; and/or (b) shuffling a repertoire of V-domains to the VH- or VL-domains of said B cells/antigen binders (e.g. as described in Raum, 2001, Cancer Immunol Immunother 50, 141-150), such that one or more modified antibodies is/are obtained; and/or (c) grafting at least one CDR of at least one of the cloned VH- and/or VL-domain/s of said B cells/antigen binders into the corresponding position/s of the variable regions of a first antibody library (e.g. principle of inserting predetermined CDR/s into the context of human antibody libraries e.g. as described in Rader, 1998, PNAS 95, 8910-5; Steinberger, 2000, J Biol Chem 17, 36073-8); such that a second antibody library is obtained; and (d) subjecting the resulting modified antibody/ies and/or antibody library to further selection on the antigen or parts thereof (e.g. for the purpose of affinity maturation) using a biological display system such as e.g. phage display, ribosomal display, bacterial display or yeast display etc. The antigen or parts thereof used for selection may be expressed (naturally or via transfection) on cells or isolated from a natural source or produced as recombinant protein or synthesized as peptide.

Mutations as described in (a) may occur in non-CDR segments or CDR segments. Said mutations include amino acid substitutions, which increase the affinity of the antigen binder (affinity maturation), which increase the stability of the antigen binder, or which increase the production rate of the antigen binder in a certain host like e.g. E. coli, yeast or mammalian cells. The amino acid substitutions may for example be achieved by using error prone PCR (Hawkins, 1992, J. Mol. Biol. 226, 889).

The term “isolated B cells” refers to single B cells, which recognize/interact or bind with a chosen antigen that was used for isolation of the B cells. The isolated B cells express and comprise antigen binders/antigen binding molecules, which in particular, recognize or interact with said antigen(s). These antigen binders on their part can be cloned, further subcloned and modified as described resulting in antibodies or fragments thereof (such as VH, VL, Fv, Fab, Fab′, F(ab′)₂, scFvs, or other antigen-binding partial sequences of antibodies) or derivatives thereof e.g. bispecific single chain antibody constructs.

The term “bispecific single chain antibody construct” relates to a construct comprising a first and a second antibody derived binding domain, preferably scFvs. The term “single-chain” as used in accordance with the present invention means that said first and second domain of the bispecific single chain construct are covalently linked, preferably in the form of a co-linear amino acid sequence encoded by a single nucleic acid molecule. It is of note that such a construct may comprise, in addition to the first and second domain (an) additional domain(s), e.g. for the isolation and/or preparation of recombinantly produced constructs. The intramolecular orientation of the V_{H}-domain and the V_{L}-domain, which are linked to each other by a linker-domain, in the scFv format is not decisive for the single chain antibodies or bispecific single chain constructs. Thus, scFvs with both possible arrangements (V_{H}-domain-linker domain-V_{L}-domain; V_{L}-domain-linker domain-V_{H}-domain) are particular embodiments of recited single chain antibody formats.

A “repertoire of V-domains” circumscribes a multitude of antibody variable (V)-domains representing a high level of sequence diversity. Such a repertoire of V-domains can be derived from naturally expressed antibody sequences isolated from e.g. blood, bone marrow or spleen as natural source (Raum et al., 2001, Cancer Immunol Immunother 50, 141-150). It can also be derived from a non-natural such as a synthetic source. Such a large number of V-domains is, after cloning, represented in a library such as a combinatorial antibody library, which then can be further used for in vitro selection.

“Shuffling” stands for a procedure of mixing VL and/or VH domains or fragments thereof. For the shuffling of e.g. a human light chain repertoire to a human heavy chain repertoire, fragments of light chain encoding DNA sequence can be e.g. PCR-amplified and cloned into the human heavy chain library using appropriate restriction enzymes (Raum et al., 2001, Cancer Immunol Immunother 50, 141-150).

“Grafting” describes a process of transferring/copying (a) sequence(s) from one sequence environment into another homologous sequence environment, for example, a CDR sequence from a donor V-region into an acceptor V region framework. This grafting technique may for example be used for humanization of mouse, rabbit or other non-human antibodies, scFvs or the like by transferring one or several CDR(s) of the non-human antibody into a human framework (Rader, 1998, PNAS 95, 8910-5; Steinberger, 2000, J Biol Chem 17, 36073-8).

In the context of “grafting”, preferably CDR grafting, “corresponding position” means the conservation of the functional arrangement of the grafted donor sequence within the acceptor sequence, e.g. CDR3 of the heavy chain is grafted between framework region three and four of the “acceptor V-sequence environment”, therefore maintaining its contribution to antigen binding in the grafted antibody, scFv or the like.

In a further preferred embodiment, the method of the present invention further comprises the step of expressing said V-domains in an expression system. In a particularly preferred embodiment of the method of the present invention said expression system is of eukaryotic origin. Advantageously, eukaryotic expression systems from yeasts, insects or bacteria, and more preferred from mammals are employed. Such expression systems are commercially available, e.g., from Stratagene or Promega .

In a most preferred embodiment the method of the present invention further comprises the step of generating antibodies or fragments or derivatives from said V-domains. Such derivatives may also comprise a construct comprising a single chain antibody and an effector molecule such as a chemokine, or cytokine, or structural protein and a linker amino acid sequence.

In another preferred embodiment the method of the present invention comprises after generation of antibodies or fragments thereof an additional protein purification step. This protein purification step may include but is not limited to a cation exchange chromatography, a gel filtration and a protein quantification step. But also other protein purification procedures like anion exchange chromatography, immobilized metal affinity chromatography (IMAC) or protein L affinity chromatography or a combination of these procedures may be employed.

These antibodies, fragments or derivatives may, possibly after further manipulations, in particular by recombinant DNA technologies to improve their binding specificity, avidity, half life etc. or to reduce their potential residual antigenicity then be formulated into pharmaceutical compositions or kits.

The pharmaceutical composition produced in accordance with the above may further comprise a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 106 to 1012 copies of the DNA molecule. The compositions may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition may comprise further agents such as interleukins or interferons depending on the intended use of the pharmaceutical composition.

In a particularly preferred embodiment the method of the present invention further comprises the steps of rearranging all possible combinations of different V_{H} and V_{L} domains. Thus, VH and VL domains deriving from different B cells can be combined in order to achieve a higher antibody diversity. In addition, the binding affinity and/or avidity of the antibody can possibly be improved.

In a further preferred embodiment, the VH- and VL-domains are specific for CD28. In a most preferred embodiment, the VH- and/or VL-domains (a) comprise (an) amino acid sequence(s) selected from the group consisting of SEQ ID Nos: 78, 80, 82, 84, 86 and 88; and/or (b) are encoded by (a) nucleic acid sequence(s) comprising sequences selected from the group consisting of SEQ ID NOs: 60, 61, 79, 81, 83, 85, 87 and 89. The sequences with the SEQ ID NOs: 60 and 61 are the originally isolated VH- and VL-domains, respectively. The sequence with SEQ ID NO: 76 is the amino acid sequence of a scFv fragment according to the invention, and the sequence with SEQ ID NO: 77 is the nucleic acid sequence encoding said fragment. The sequences with SEQ ID NOs: 78, 80, 82, 84, 86 and 88 are the amino acid sequences of CDRs according to the invention, and the sequences with SEQ ID NOs: 79, 81, 83, 85, 87 and 89 are the corresponding nucleic acid sequences.

In a further preferred embodiment, the VH- and VL-domains are specific for the murine Ig part of a fusion protein like the recombinant fusion protein of human CD28 and murine Ig. (recCD28-murine Ig/rCD28) or human. CD40 and murine Ig. In a most preferred embodiment, the VH- and/or VL-domains (a) comprise (an) amino acid sequence(s) selected from the group consisting of SEQ ID Nos: 64, 66, 68, 70, 72 and 74; and/or (b) are encoded by (a) nucleic acid sequence(s) comprising sequences selected from the group consisting of SEQ ID NOs: 58, 59, 65, 67, 69, 71, 73 and 75. The sequences with the SEQ ID NOs: 58 and 59 are the originally isolated VH- and VL-domains, respectively. The sequence with SEQ ID NO: 62 is the amino acid sequence of a scFv fragment according to the invention, and the sequence with SEQ ID NO: 63 is the nucleic acid sequence encoding said fragment. The sequences with SEQ ID NOs: 64, 66, 68, 70, 72 and 74 are the amino acid sequences of CDRs according to the invention, and the sequences with SEQ ID NOs: 65, 67, 69, 71, 73 and 75 are the corresponding nucleic acid sequences.

In a further preferred embodiment, the method of the present invention further comprises the step of generating, bispecific antibody constructs or single chain antibodies.

The term “single chain antibody” refers to an antibody containing one binding specificity for a (preferably predefined) epitope. Single chain antibodies comprise one VL and one VH region and a linker amino acid sequence. Single chain antibodies have been described, for example, in Bejcek, 1995, Cancer Research 55, 2346-2351.

The term “bispecific antibody construct” refers to a construct that comprises two different binding specificities for (preferably predefined) different epitopes and optionally different antigens. Bispecific antibody constructs have been described, for example, in Mack, 1995, PNAS 92, 7021-7025.

In a further preferred embodiment, said derivatives comprise at least one binding site specific for CD28.

In a more preferred embodiment, said derivatives (a) comprise the amino acid sequence as set forth in SEQ ID NO: 76; and/or (b) are encoded by a nucleic acid sequence comprising the sequence as set forth in SEQ ID NO: 77.

In a further preferred embodiment, said derivatives comprise at least one binding site specific for the murine Ig part of a fusion protein like the recombinant fusion protein of human CD28 and murine Ig (recCD28-murine Ig/rCD28) or human CD40 and murine Ig.

In a more preferred embodiment, said derivatives (a) comprise the amino acid sequence as set forth in SEQ ID NO: 62; and/or (b) are encoded by a nucleic acid sequence comprising the sequence as set forth in SEQ ID NO: 63.

In an additional preferred embodiment the method of the present invention further comprises an assay for antibody evaluation. To verify the binding specificity of the antibodies evaluation assays and preferably binding assays may be performed. These binding assays advantageously use the initial fishing antigen or an equivalent thereof. Assays such as ELISA, FACS-based assays, BIAcore™, or dot blot may then be performed.

In an alternative embodiment, the present invention relates to an antibody generated by the method of the invention, which is specific for human CD28.

In a preferred embodiment, said antibody is generated by any of the methods according to the invention, wherein said antibody (a) comprises (an) amino acid sequence(s) selected from the group consisting of SEQ ID NOs: 76, 78, 80, 82, 84, 86 and 88; and/or (b) is encoded by (a) nucleic acid sequence(s) comprising sequences selected from the group consisting of the SEQ ID NOs: 60, 61, 77, 79, 81, 83, 85, 87 and 89.

In an alternative embodiment, the present invention relates to an antibody generated by the method of the invention, which is specific for the murine Ig part of a fusion protein like the recombinant fusion protein of human CD28 and murine Ig (recCD28-murine Ig/rCD28) or human CD40 and murine Ig.

In a preferred embodiment, said antibody is generated by any of the methods according to the invention, wherein said antibody (a) comprises (an) amino acid sequence(s) selected from the group consisting of SEQ ID NOs: 62, 64, 66, 68, 70, 72 and 74; and/or (b) is encoded by (a) nucleic acid sequence(s) comprising sequences selected from the group consisting of the SEQ ID NOs: 58, 59, 63, 65, 67, 69, 71, 73 and 75.

Additionally, the present invention relates to a device for assessing the presence of a detectable signal as defined in the method as described above, wherein said device comprises a closed system for the detection laser-beam and a catcher tube, and wherein the B cell of interest can be collected as a single cell by means of an electrochemical device which is triggered by an electric signal generated by the FACS device, wherein the electrochemical device moves the nozzle of the steady catcher tube liquid stream for a programmed time over a collecting tube, microtiter plate or other container after a B cell is sorted.

In the device described in U.S. Pat. No. 5,326,696 the cells of interest are singled out in drops. Subsequently, the emission is measured and the drops containing the cells of interest are deflected by means of an electrochemical device. However, the method of the invention does not properly function using this device since the signal obtained by measuring single drops is qualitatively not sufficient for use in the method of the invention due to manifold scattering and light loss. Thus, the present invention, including the signal generation and detection, is advantageously carried out in a solid fluid stream, wherein the cells are collected directly from said liquid stream without being singled out beforehand.

Preferred embodiments of this method of the invention include those that have been detailed in accordance with the method of the invention that has been characterized herein above. These preferred embodiments apply mutatis mutandis to this embodiment of the invention.

The figures show:

FIG. 1:

Schematic drawing representing the detection principle of autoreactive B cells by FACS sort using FRET as selection principle. [symbol] Spotted stars represent a second label like fluorescein coupled to a receptor like anti-IgD antibody, which specifically binds to a surface immunoglobulin molecule on B cells. [symbol] White stars represent a first label like Alexa Fluor 546 coupled to an antigen of interest, which is not activated due to lack of spatial proximity to the second donor label. [symbol] Black stars represent a first label like Alexa Fluor 546 coupled to an antigen of interest, which is activated by the second donor label, since antigen and receptor have bound closely together on the same surface immunoglobulin molecule.

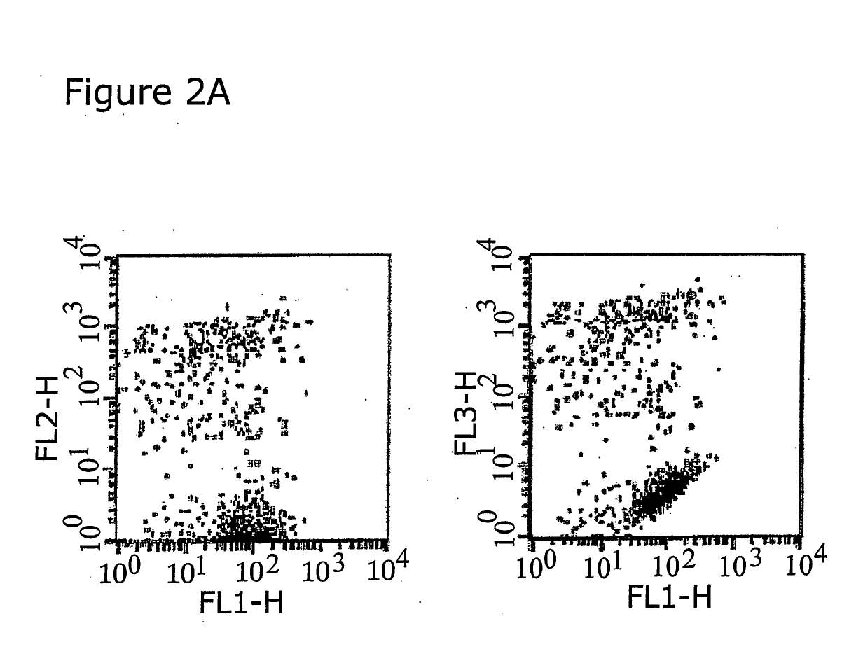

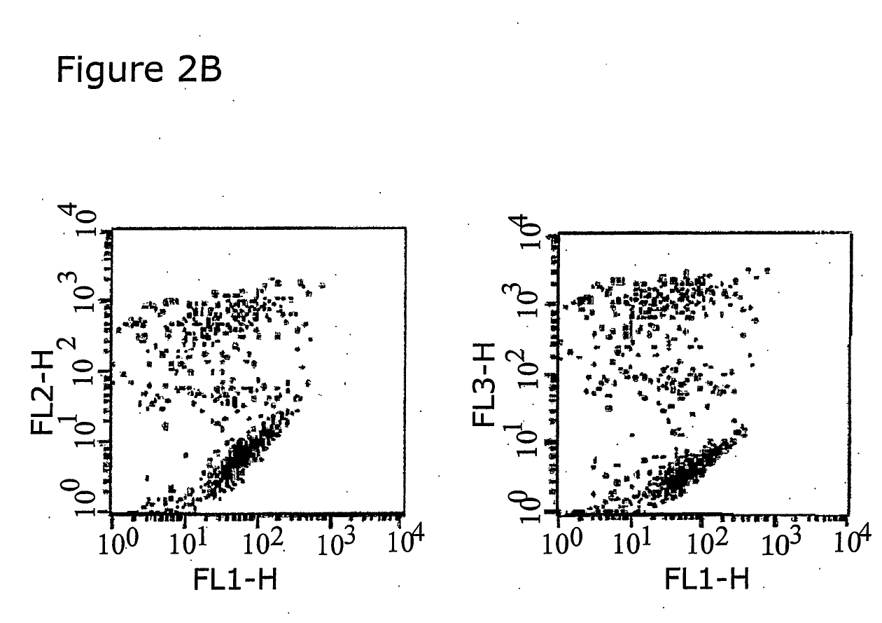

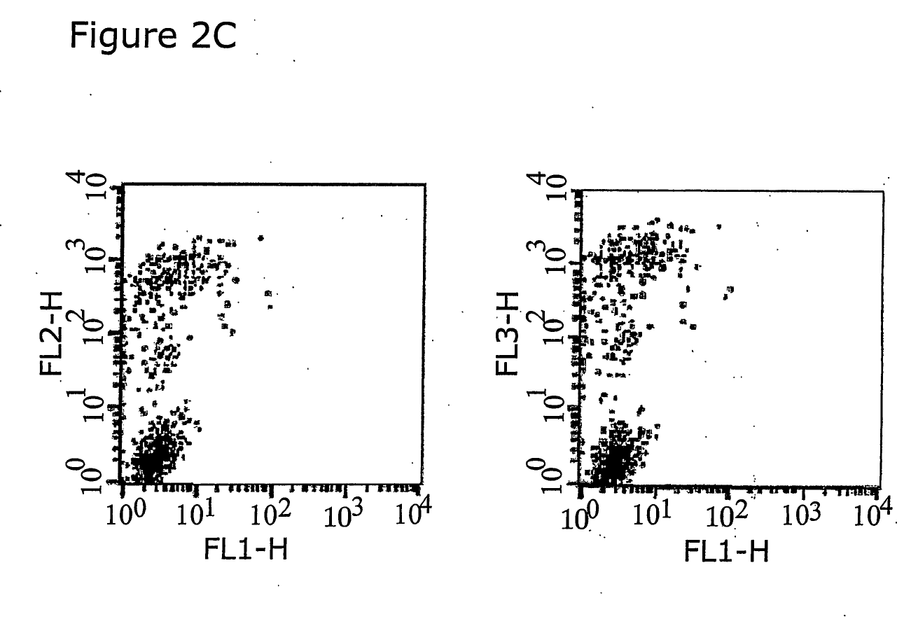

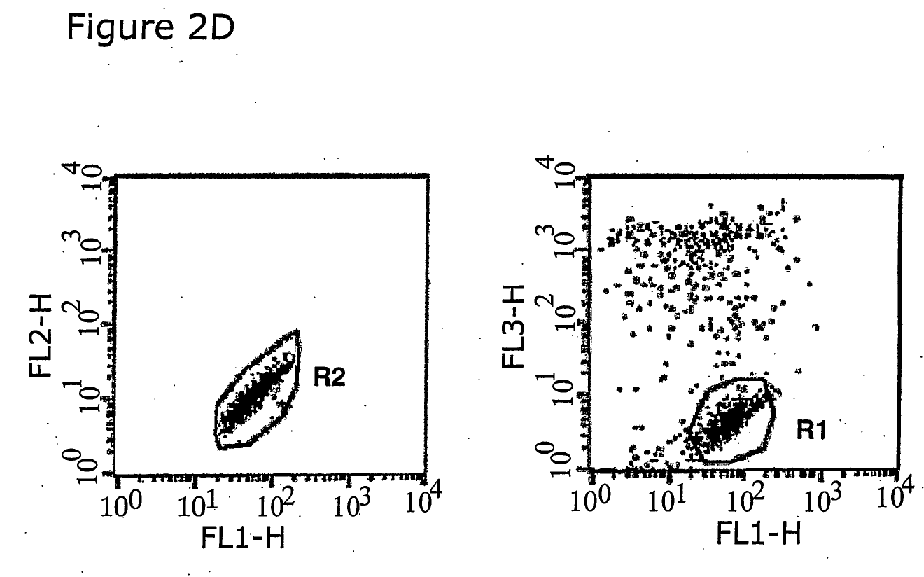

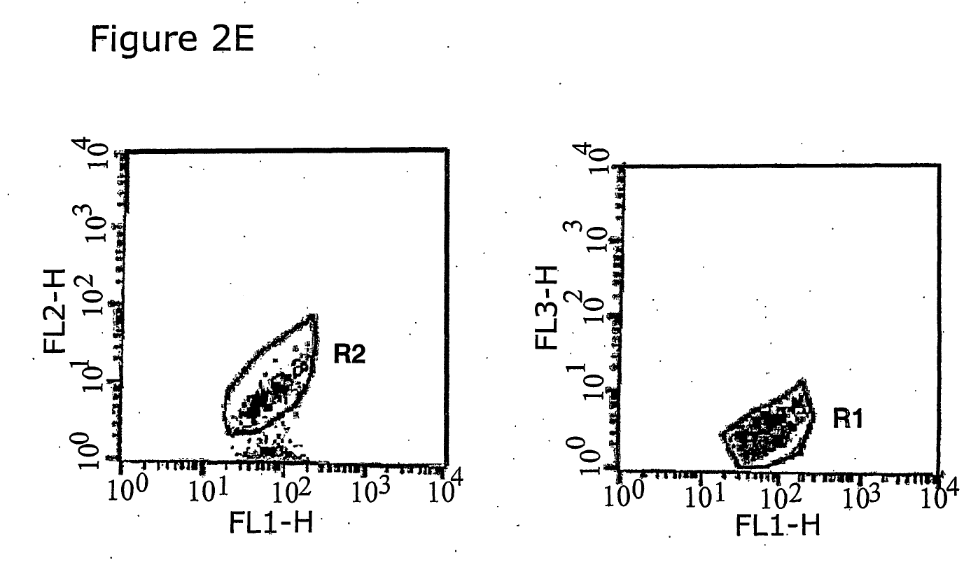

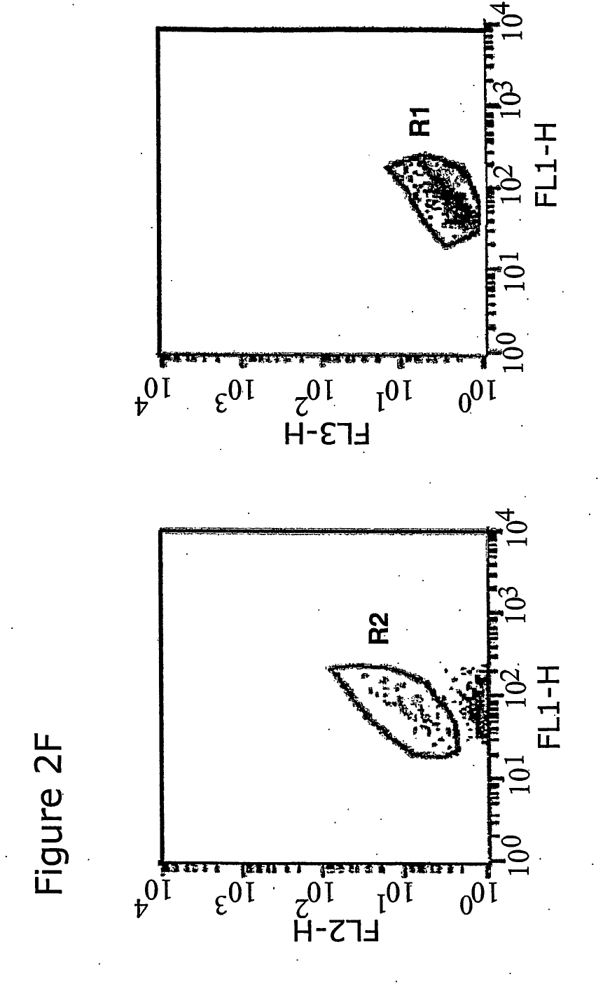

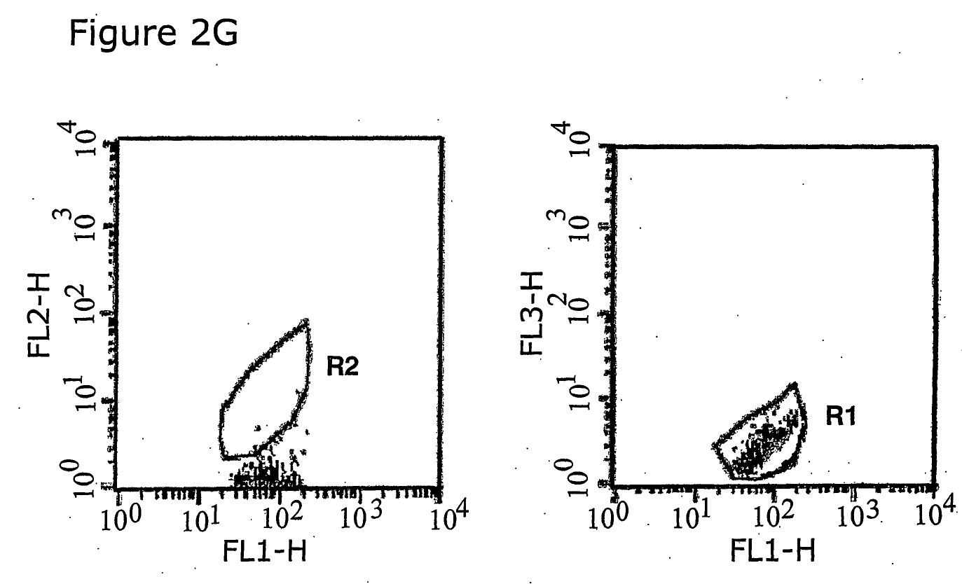

FIG. 2:

FACS images showing the selection of single B cells from a mouse B cell line mixture using FRET. A) 8.18C5 mouse cells stained with donor-fluorochrome fluorescein anti IgG FITC and propidium iodide (PI), amplification FL2: 490. B) 8.18C5 mouse cells stained with anti IgG FITC and MOG Fc Alexa Fluor 546 and PI showed real FRET signal, amplification FL2: 490. C) 8.18C5 mouse cells stained with MOG Fc Alexa Fluor 546 and PI as FL2 control, amplification FL2: 490. D) 8.18C5 mouse cells stained with anti IgG FITC and MOG Fc Alexa Fluor 546 and PI with increased FL2 amplification of 500. E) 8.18C5 (160 μl) and A20 mouse cells mixed and both double stained with anti IgG FITC and MOG Fc Alexa Fluor 546 and PI, amplification FL2: 500. F) as E, but with lowered mixing ratio 8.18C5 (40 μl) to A20. G) A20 double stained mouse cells anti IgG FITC and MOG Fc Alexa Fluor 546 and PI, amplification FL2: 500. R1: gate on living Ig positive (FL1 positive) cells; R2: FRET gate on living cells.

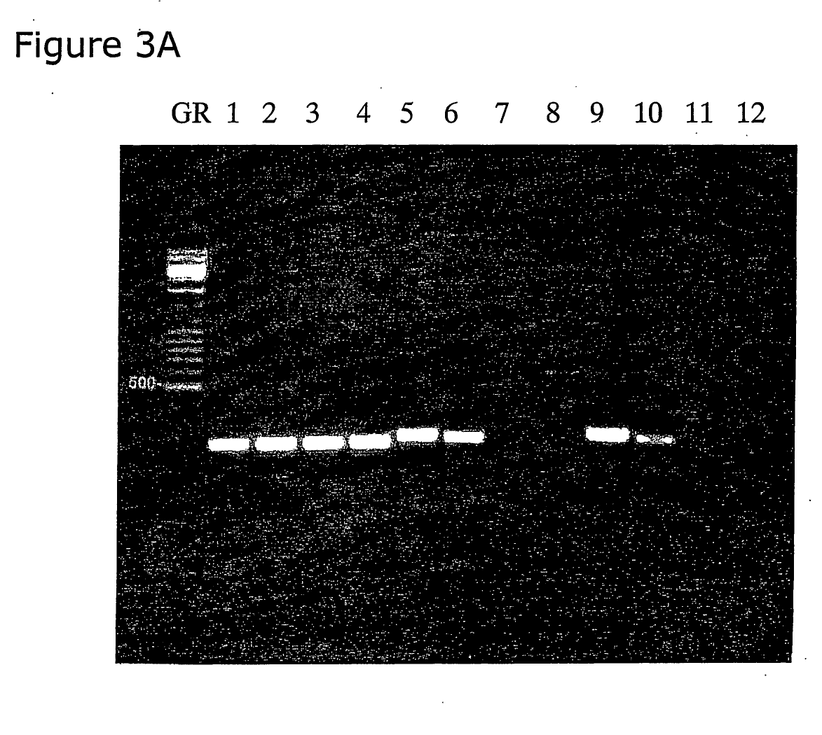

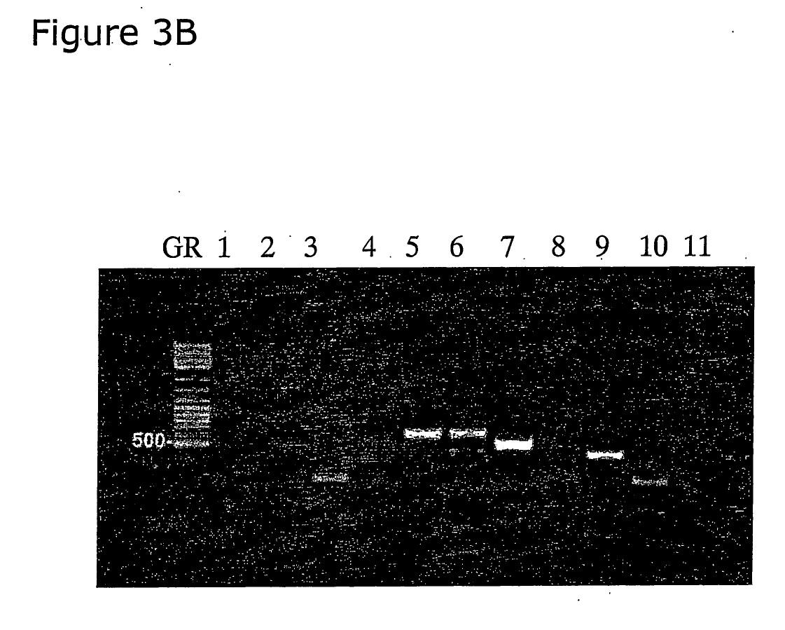

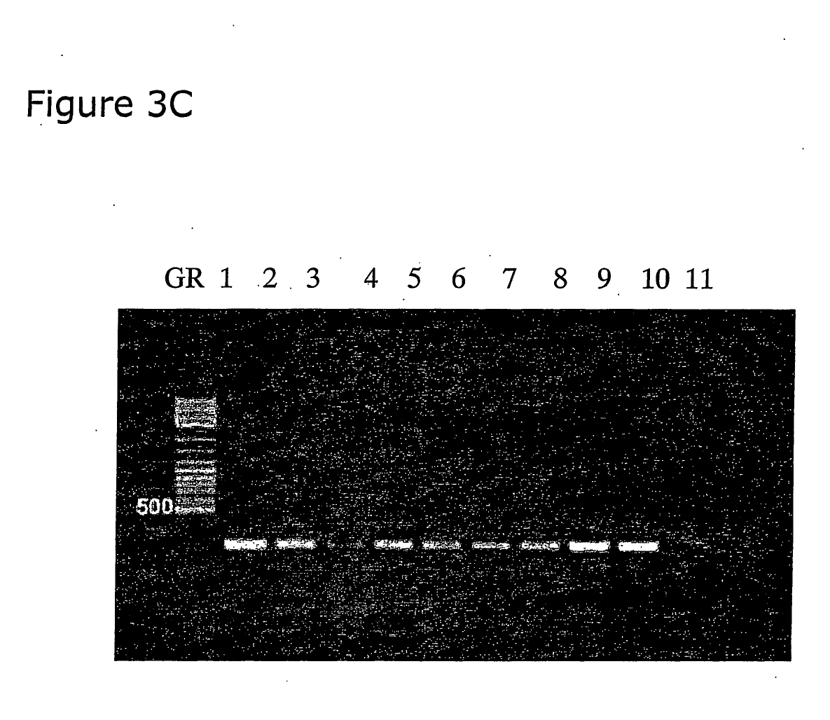

FIG. 3:

A) Analytical agarose gel electrophoresis to test A20- and MOG-primers specificity. GR) Size standard marker (GeneRuler™ DNA ladder Mix, MBI Fermentas, St. Leon-Rot, Germany). 1) CDNA A20 cells preparation 1 plus 5′ muB-actin primer and 3′ muB-actin primer. 2) cDNA A20 cells preparation 2 plus 5′ muB-actin primer and 3′ muB-actin primer. 3) CDNA MOG cells preparation 1 plus 5′ muB-actin primer and 3′ muB-actin primer. 4) cDNA MOG cells preparation 2 plus 5′ muB-actin primer and 3′ muB-actin primer. 5) cDNA A20 cells preparation 1 plus 5′ VH-A20-outside primer and 3′ VH-A20-outside primer. 6) cDNA A20 cells preparation 1 plus 5′ VH-A20-inside primer and 3′ VH-A20-inside primer. 7) CDNA A20 cells preparation 1 plus 5′ VH-MOG-outside primer and 3′ VH-MOG-outside primer. 8) cDNA A20 cells preparation 1 plus 5′ VH-MOG-inside primer and 3′ VH-MOG-inside primer. 9) cDNA MOG cells preparation 1 plus 5′ VH-MOG-outside primer and 3′ VH-MOG-outside primer. 10) cDNA MOG cells preparation 1 plus 5′ VH-MOG-inside primer and 3′ VH-MOG-inside primer. 11) cDNA MOG cells preparation 1 plus 5′ VH-A20-outside primer and 3′ VH-A20-outside primer. 12) cDNA MOG cells preparation 1 plus 5′ VH-A20-inside primer and 3′ VH-A20-inside primer. B) Analytical agarose gel electrophoresis of nested PCR products for selected mouse B cell clones using the A20-primer pairs. GR) (GeneRuler™ DNA ladder Mix, MBI Fermentas, St. Leon-Rot, Germany), lanes 1-8) were nested PCR products from 8 different single selected cells, 9) was a pool of 200 MOG cells used as negative control, 10) was a pool of 200 A20 cells used as positive control, 11) was used as reagent control. C) Analytical agarose gel electrophoresis of nested PCR products for selected mouse B cell clones using the MOG-primer pairs. GR) (GeneRuler™ DNA ladder Mix, MBI Fermentas, St. Leon-Rot, Germany), lanes 1-8) were nested PCR products from 8 different single selected cells, 9) was a pool of 200 MOG cells used as positive control, 10) was a pool of 200 A20 cells used as negative control, 11) was used as reagent control.

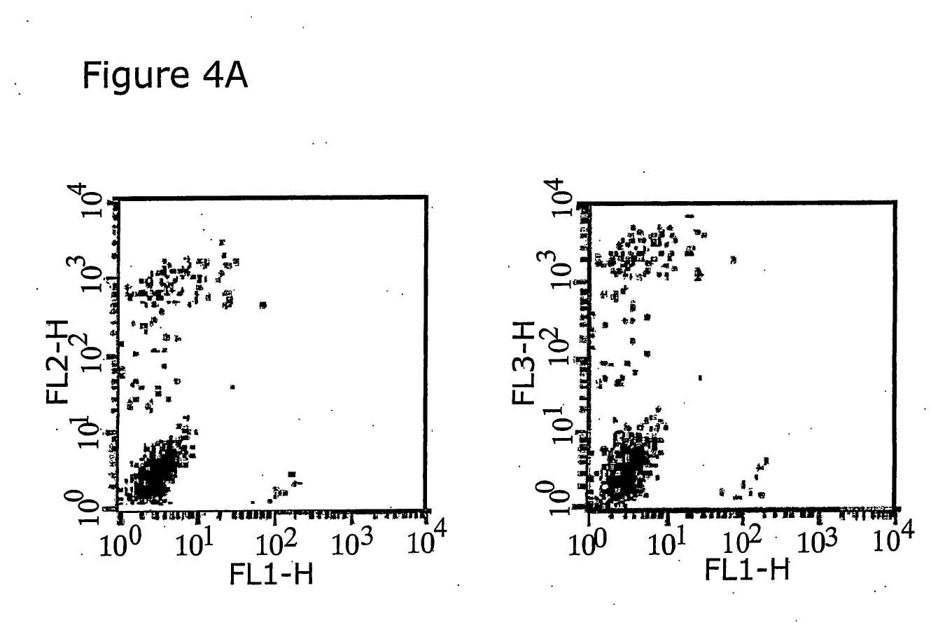

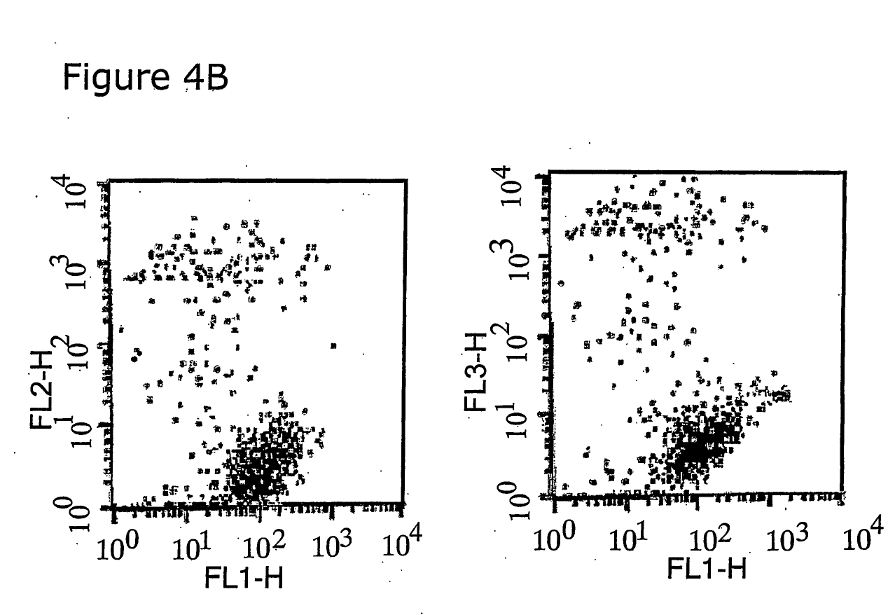

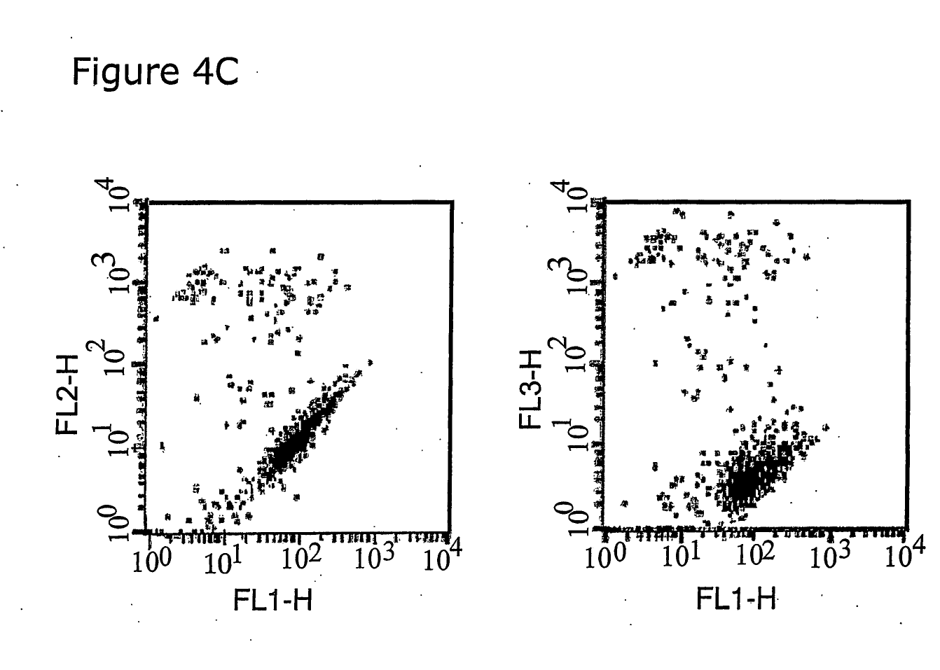

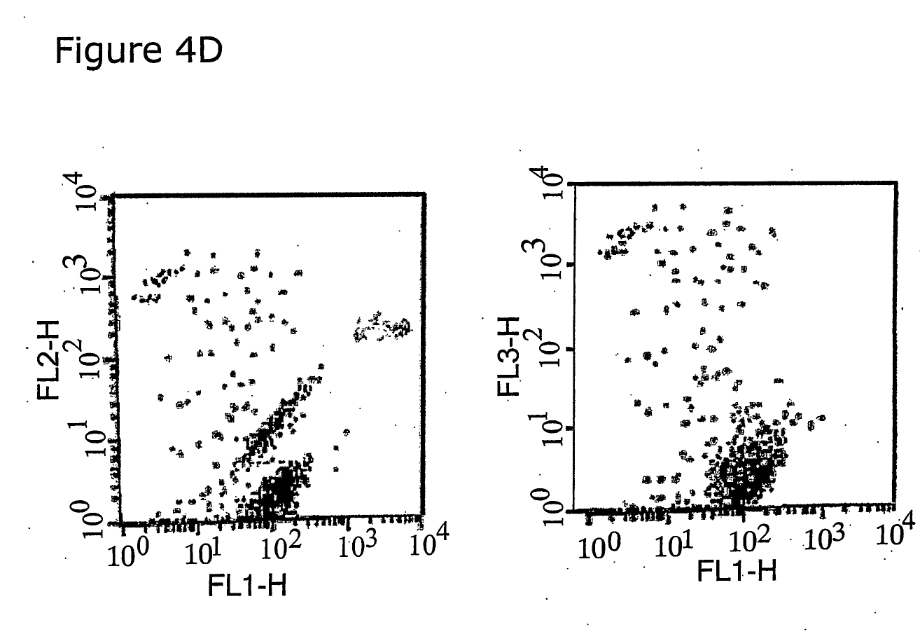

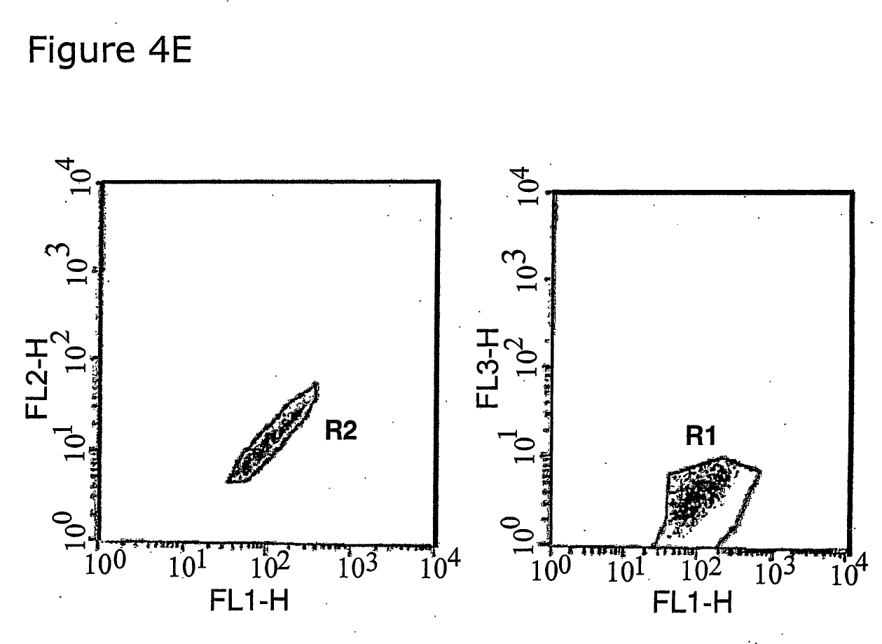

FIG. 4:

FACS images of dilution series experiments. A) 8.18C5 mouse B cells stained with MOG-Fc Alexa Fluor 546 as control reaction (labeling reaction 5). B) 8.18C5 mouse B cells stained with IgG-fluorescein as control reaction (labeling reaction 6). C) 8.18C5 mouse B cells double stained with MOG-Fc Alexa Fluor 546 and IgG-fluorescein as FRET positive control reaction (labeling reaction 7). D) Mixture of A20 mouse B cells and 8.18C5 mouse B cells double stained with MOG-Fc Alexa Fluor 546 and IgG-fluorescein 50%/50% dilution (labeling reaction 8). E) see D plus gating: gate 1=R1 on living Ig positive (FL1 positive) cells without FRET signal; gate 2=R2: FRET gate on living cells with FRET signal.

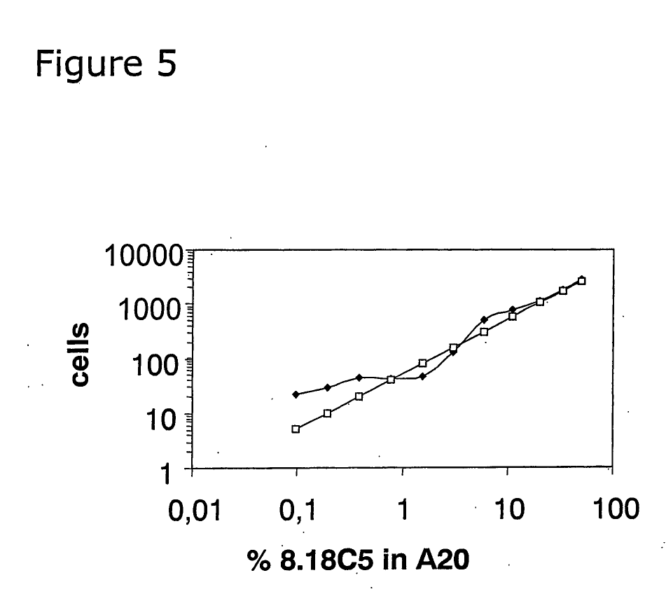

FIG. 5:

Excel graph of dilution series experiment (labeling reactions 8-18) showing the specificity of FRET selection. Squares: expected cell count. Filled diamonds: actually counted cells in FRET gate.

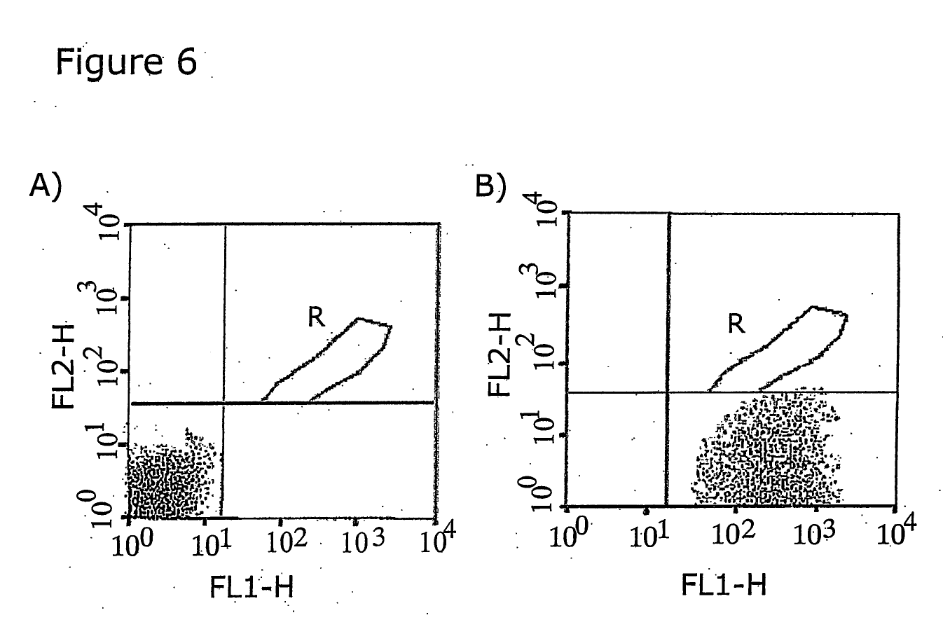

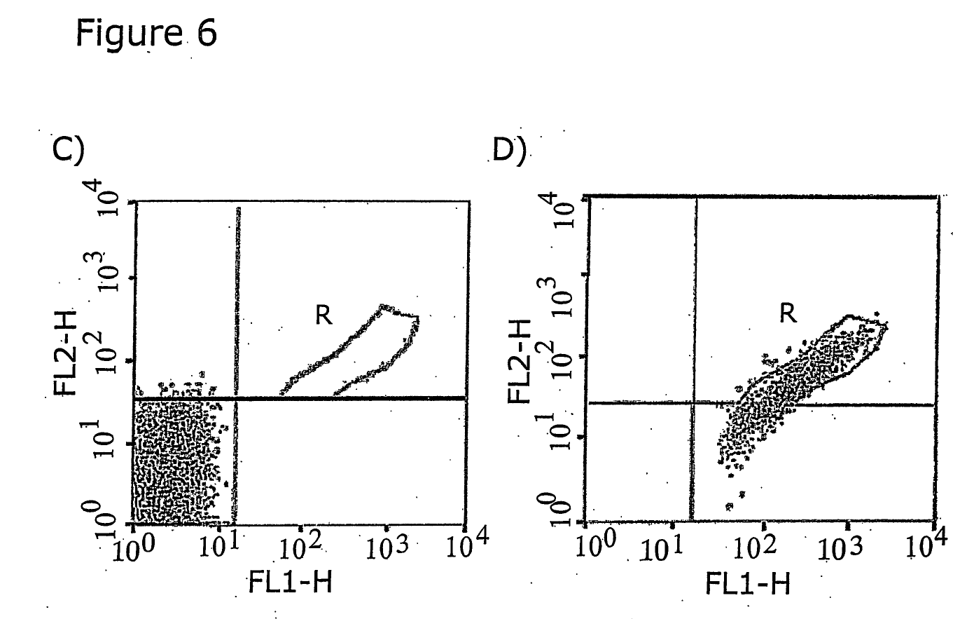

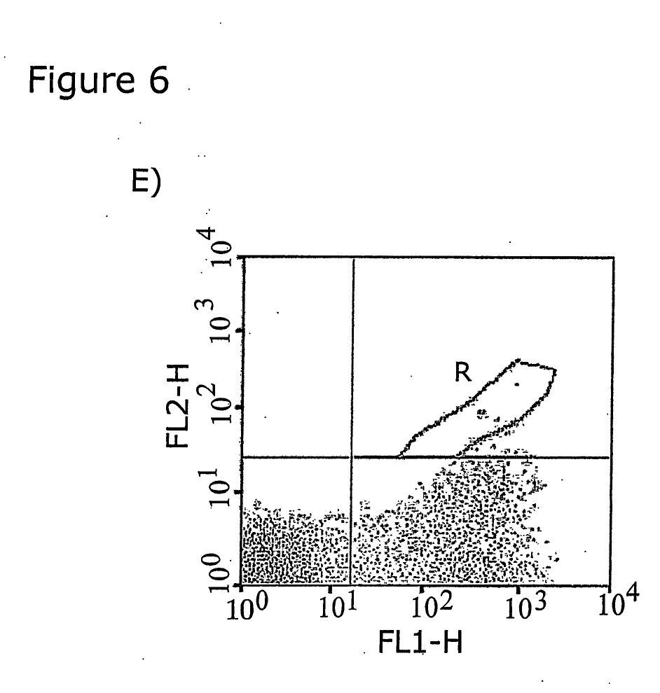

FIG. 6:

FACS images of single B cell selection from human blood using FRET. A) Unstained cells, B) cells stained with anti human IgD fluorescein, C) cells stained with anti human IgD Alexa Fluor 546, D) cells double stained with anti human IgD fluorescein and anti human IgD Alexa Fluor 546 as positive gate setting control, E) cells double stained with anti human IgD fluorescein and rCD28 Alexa Fluor 546 for real sorting. R=FRET gate.

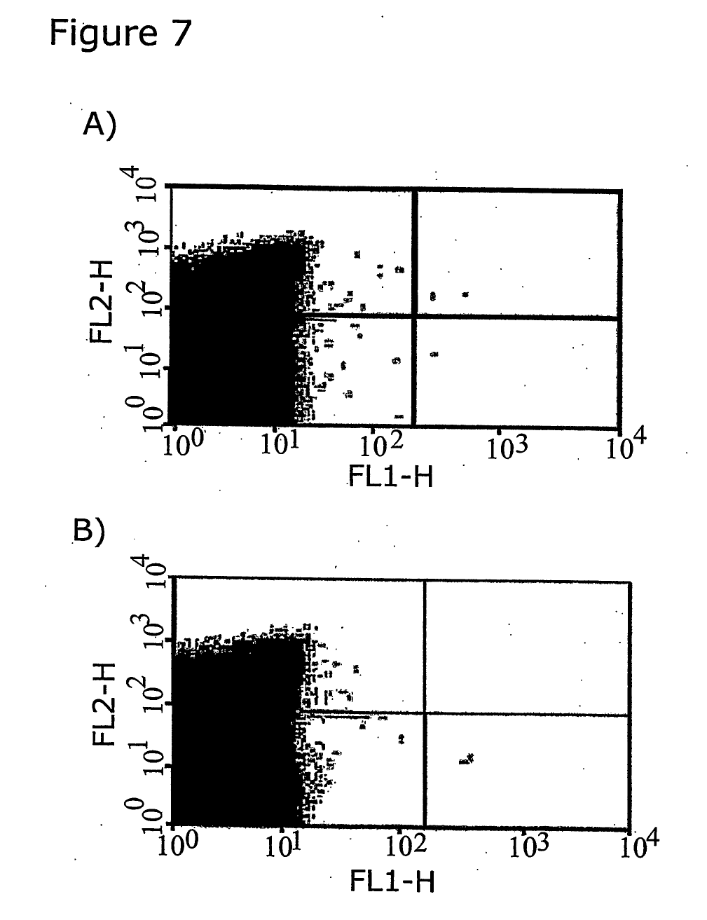

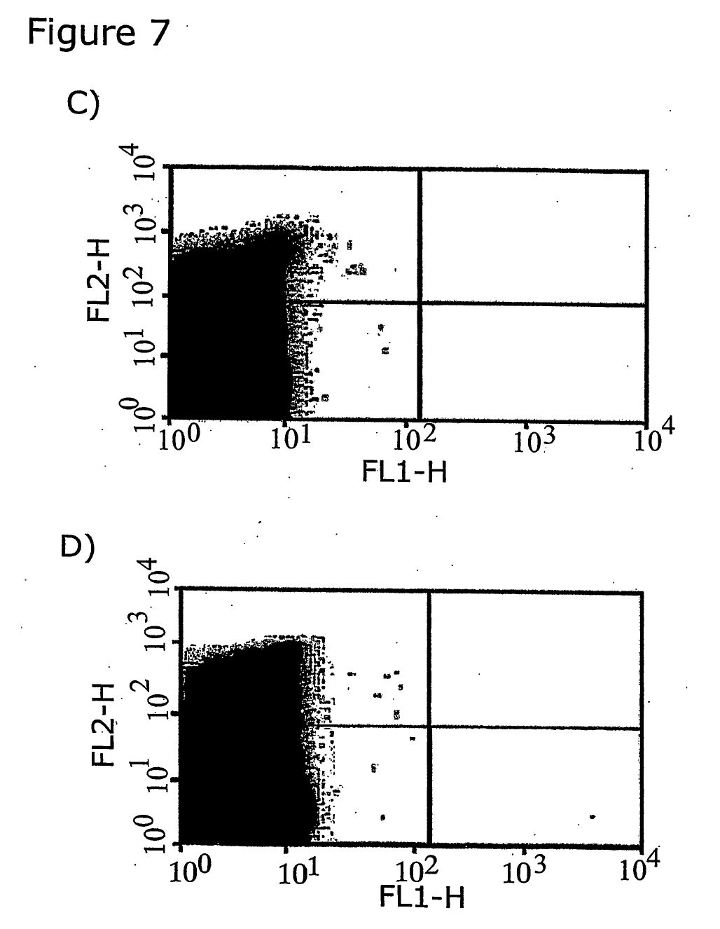

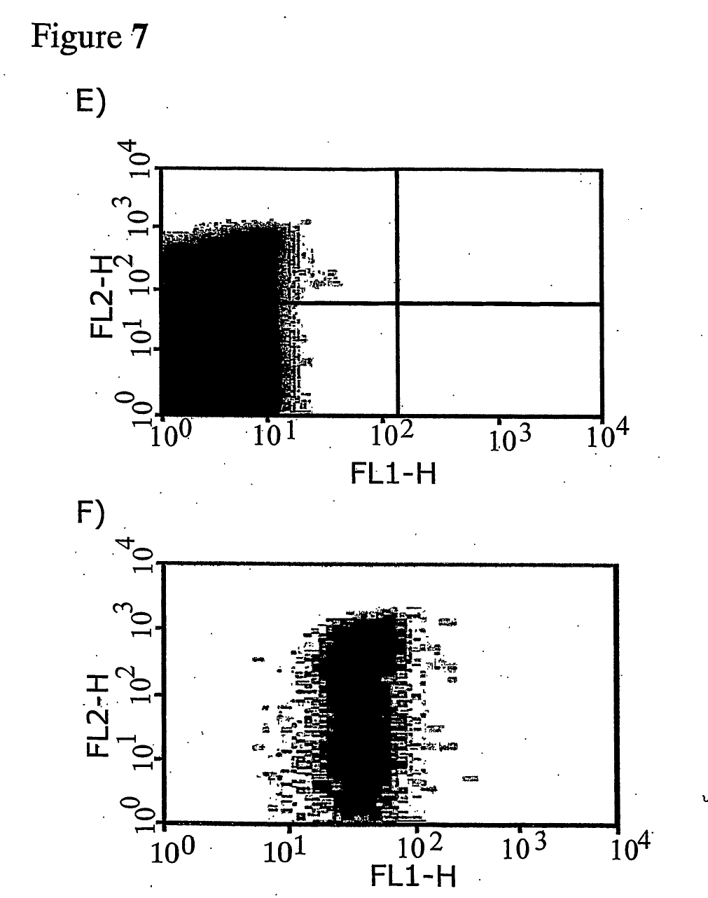

FIG. 7:

FACS images of multicolor sort of human B cells. A) Cells labeled with Cy2-EpCAM antigen 5.00 μg/ml, B) cells labeled with Cy2-EpCAM antigen 2.50 μg/ml, C) cells labeled with Cy2-EpCAM antigen 1.25 μg/ml, D) cells labeled with Cy2-EpCAM antigen 0.63 μg/ml, E) cells labeled with Cy2-EpCAM antigen 0.31 μg/ml, F) double stained cells with anti CD45-FITC and anti IgD-PE.

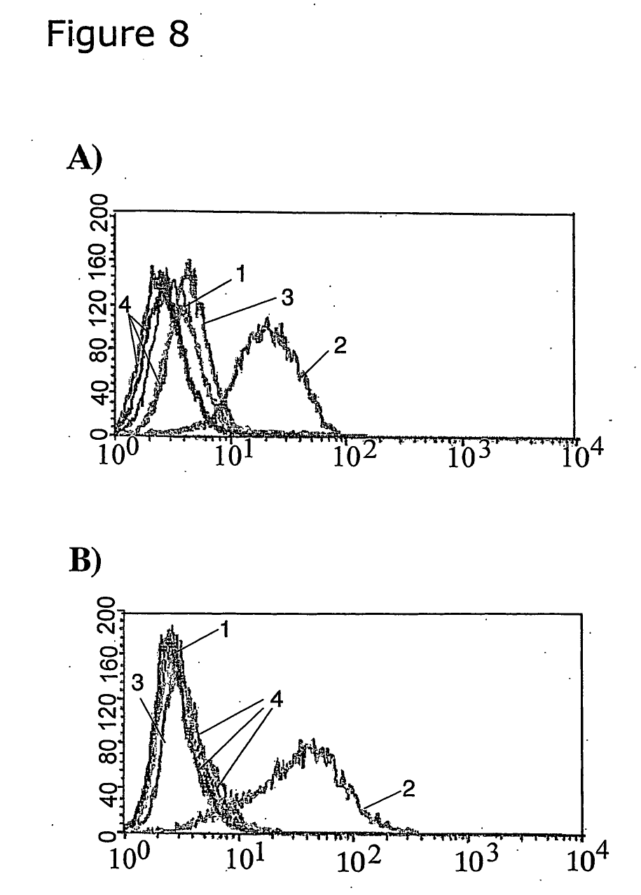

FIG. 8:

FACS-based anti-EpCAM binding assay using A) KATOIII cells as EpCAM positive cells and B) CHO 17-1A transfected cells as EpCAM positive cells. Line No. 1: anti-His tag antibody binding as negative control, Line No. 2: bispecific scFv anti EpCAM×anti CD3 binding as positive control (Mack, 1995, PNAS 92, 7021-7025), Line No. 3: anti-EGFR antibody binding, Lines No. 4: three different supernatants of anti-EpCAM scFv clones selected using multicolor FACS sorting (as described in the reference example).

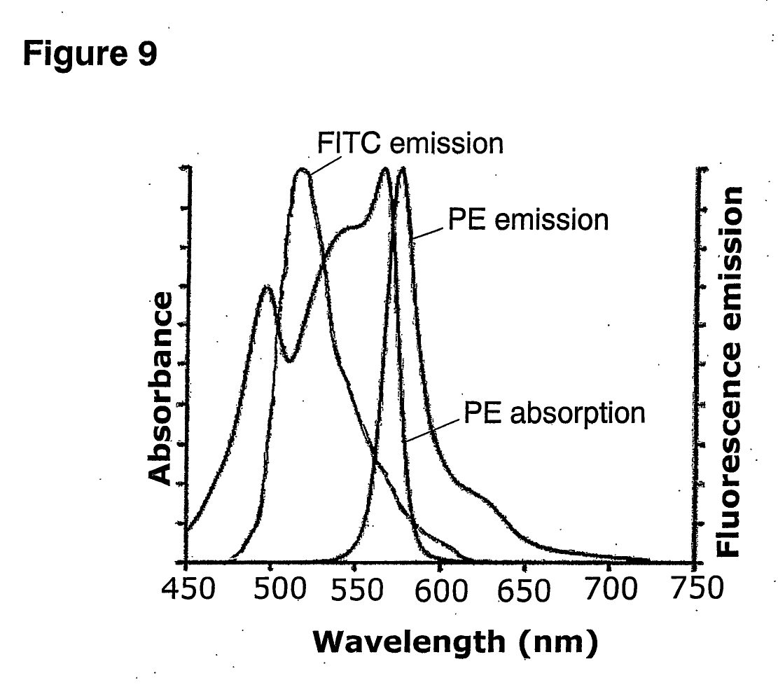

FIG. 9:

Spectrum overlay of phycoerythrin (PE) and FITC (modified from Molecular Probes online catalogue, Eugene, Oreg., USA).

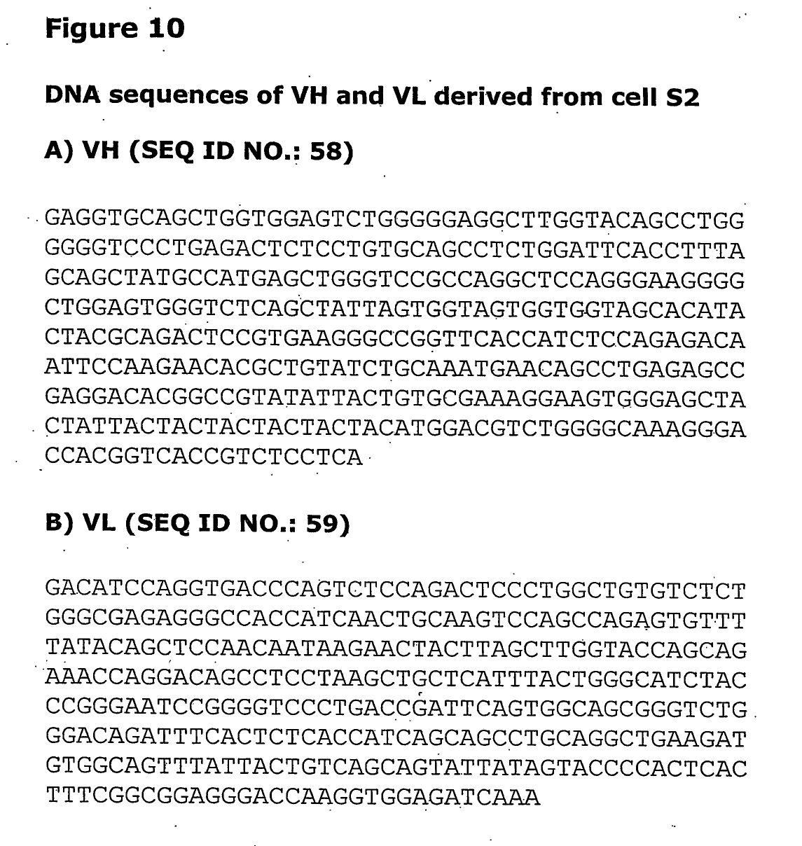

FIG. 10:

Nucleic acid sequences of VH (A) and VL (B) derived from isolated cell S2 (SEQ ID NO.: 58 and 59 respectively).

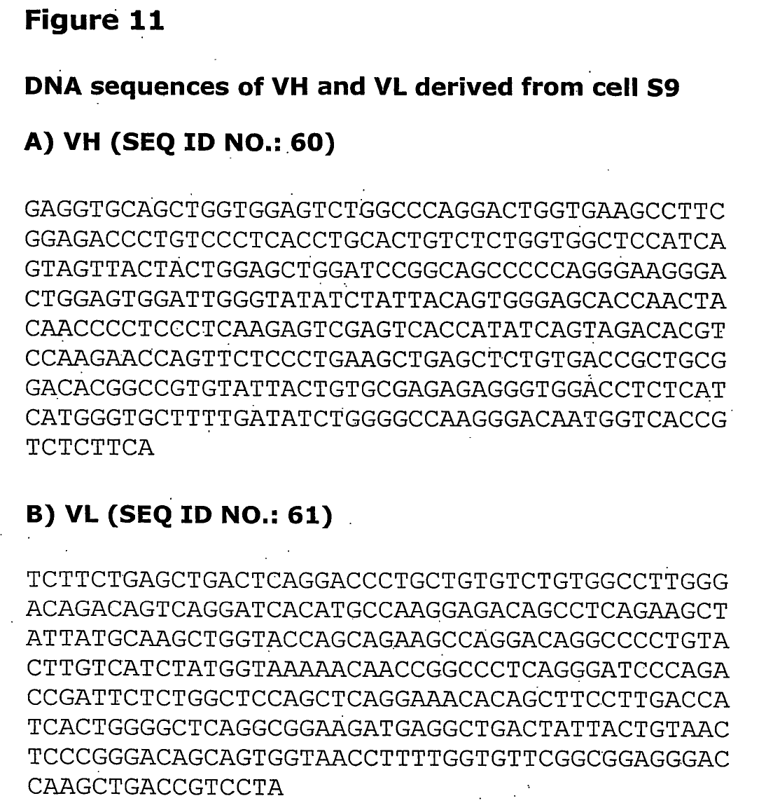

FIG. 11:

Nucleic acid sequences of VH (A) and VL (B) derived from isolated cell S9 (SEQ ID NO.: 60 and 61 respectively).

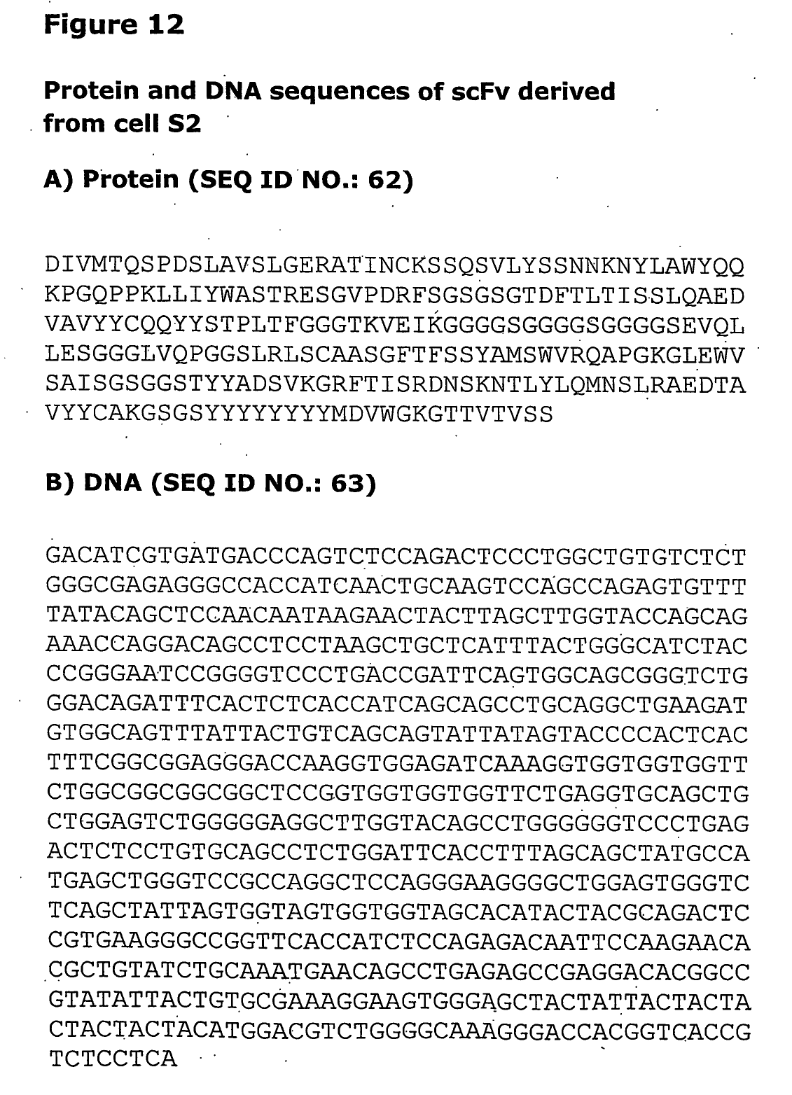

FIG. 12:

Amino acid sequence (A) and nucleic acid sequence (B) of scFv VL-VH derived from isolated cell S2 (SEQ ID NO.: 62 and 63 respectively).

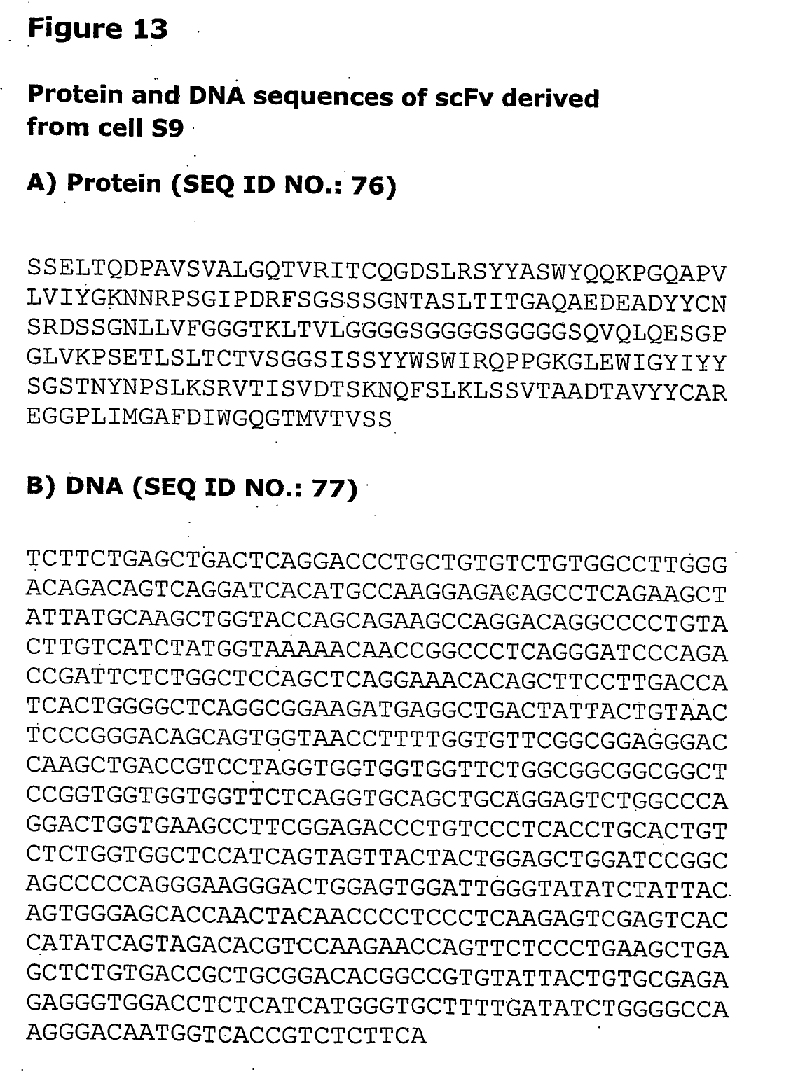

FIG. 13:

Amino acid sequence (A) and nucleic acid sequence (B) of scFv VL-VH derived from isolated cell S9 (SEQ ID NO.: 76 and 77 respectively).

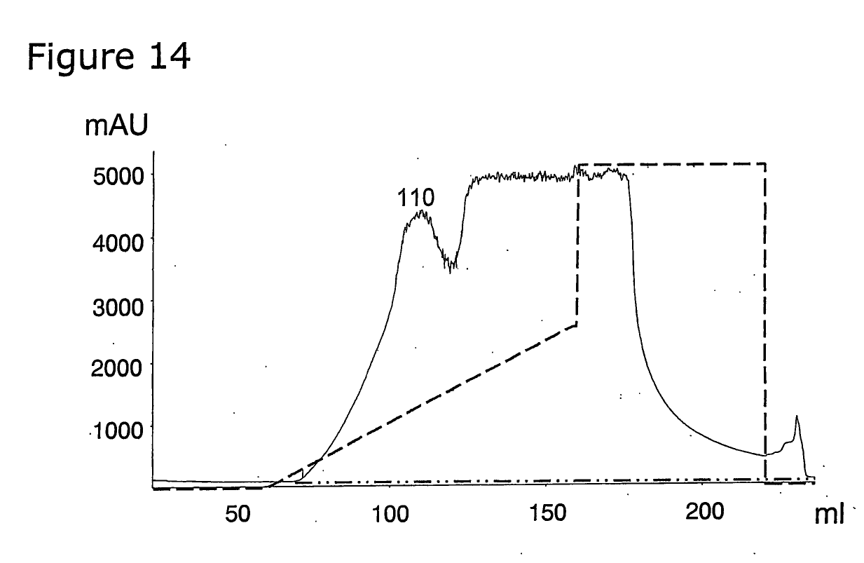

FIG. 14:

Typical elution pattern of anti-CD28 scFv containing protein from a cation exchange column measured in milli absorption units (mAU) at 280 nm. The dashed line shows the elubon gradient of buffer B. The irregularly dashed line parallel to the x-axis represents the edited baseline. The anti-CD28 scFv protein was eluted at 110 ml.

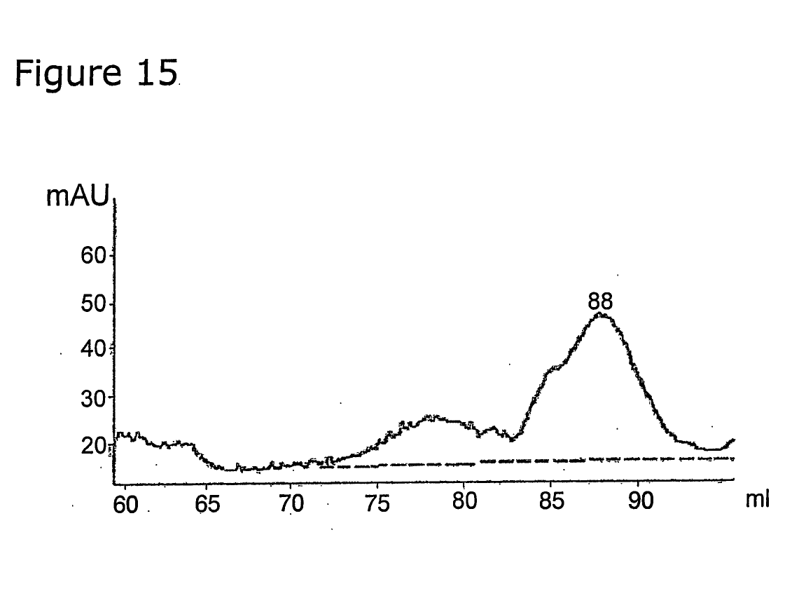

FIG. 15:

Anti-CD28 scFv protein elution pattern from a Sephadex S200 gelfiltration column. The protein peak at 88 ml corresponds to a molecular weight of approx. 27 kD and contains the anti-CD28 scFv. The dashed line parallel to the x-axis represents the edited baseline.

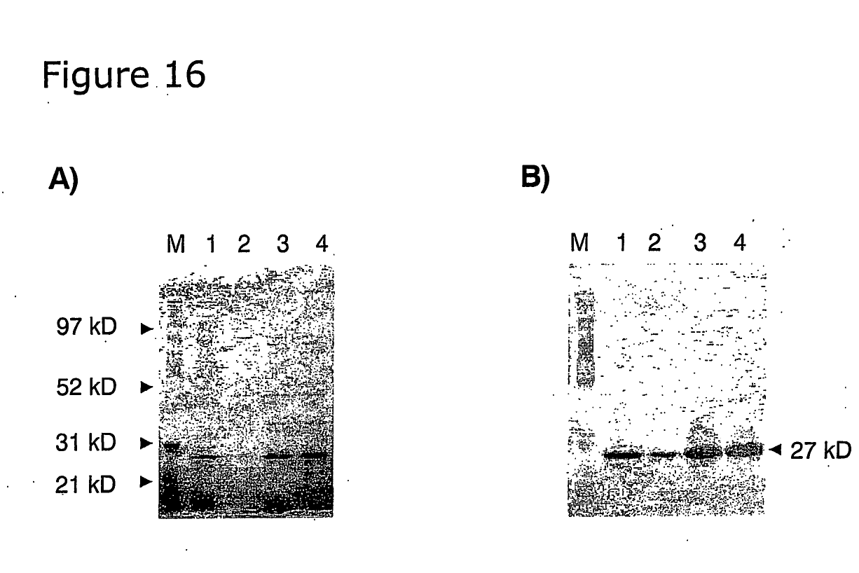

FIG. 16:

SDS-PAGE (A) and Western blot (B) analysis of anti-CD28 scFv protein from cation exchange chromatography as shown in FIG. 10. SDS-PAGE was stained with colloidal Coomassie and Western blot was incubated with Penta His antibody and goat anti-mouse antibody labeled with alkaline phosphatase. Lane 1: cell culture supernatant; lane 2: column flow trough; lane 3: anti-CD28 scFv eluate 0.2μ filtrated; lane 4: anti-CD28 scFv eluate unfiltrated, M=molecular weight marker

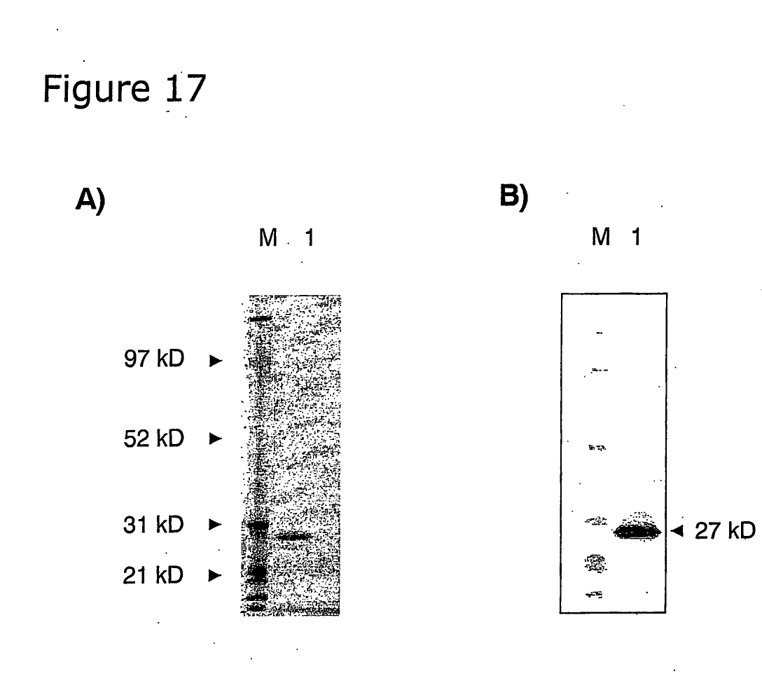

FIG. 17:

SDS-PAGE (A) and Western blot (B) analysis of anti-CD28 scFv protein from gelfiltration chromatography as shown in FIG. 11. SDS-PAGE was stained with colloidal Coomassie and Western blot was incubated with Penta His antibody and goat anti-mouse antibody labeled with alkaline phosphatase. Lane 1: purified anti-CD28 scFv; M=molecular weight marker

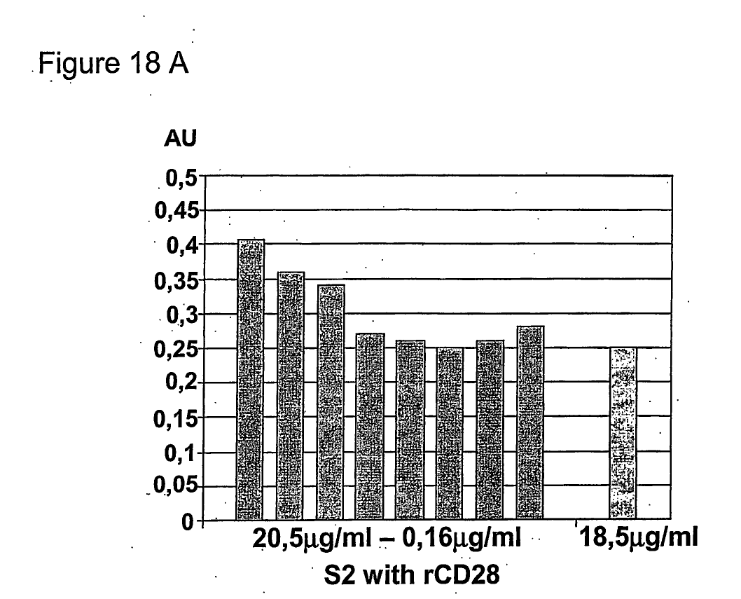

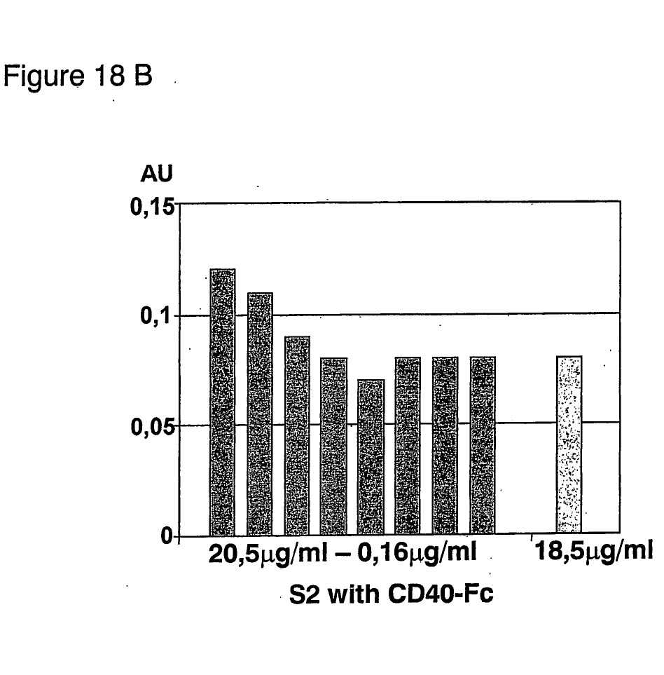

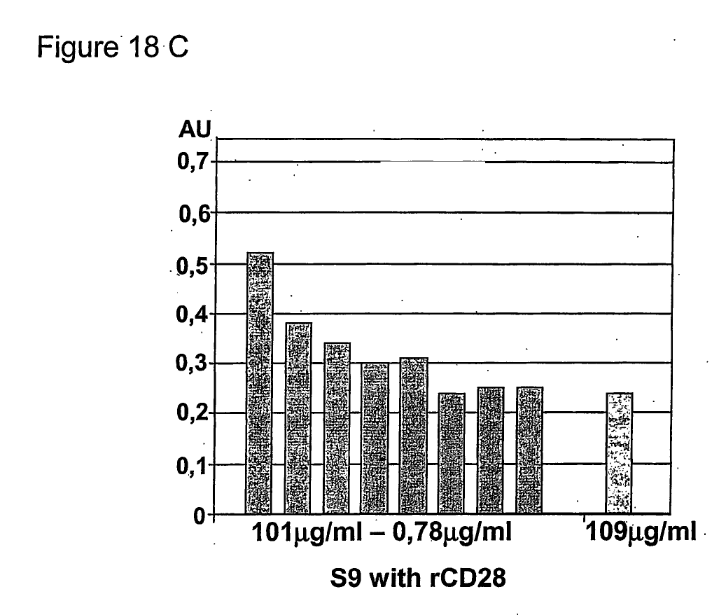

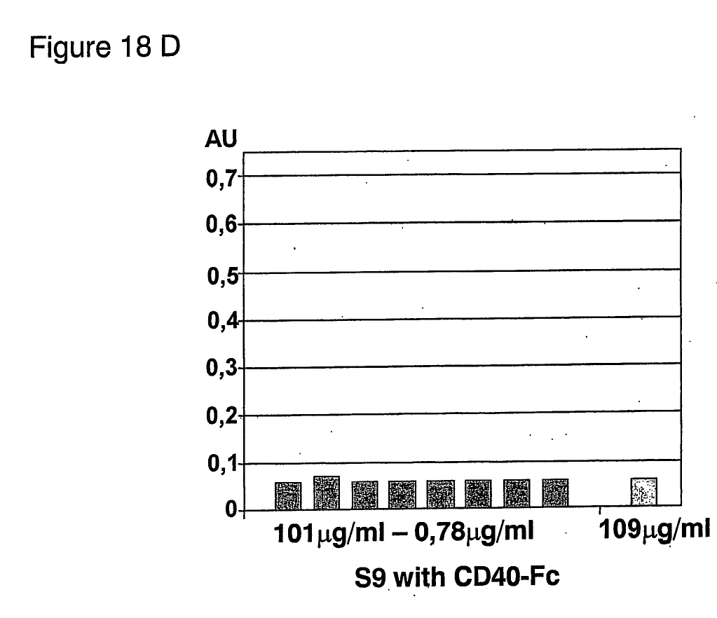

FIG. 18

ELISA binding assays. (A) Binding of the scFv antibody cloned from cell S2 to the recombinant fusion protein of human CD28-murine Ig (rCD28). The graph depicts absorption values (AU) for the scFv antibody preparation in serial twofold dilutions with concentrations in a range from 20.5 μg/ml to 0.16 μg/ml (dark grey). As a control the preparation of the scFv antibody cloned from another cell S3 lacking binding activity was used at a concentration of 18.5 μg/ml (light grey). (B) Binding of the scFv antibody cloned from cell S2 to the recombinant fusion protein of human CD40-murine Ig (CD40-Fc). The graph depicts absorption values (AU) for the scFv antibody preparation in serial twofold dilutions with concentrations in a range from 20.5 μg/ml to 0.16 μg/ml. As a control the preparation of the scFv antibody cloned from another cell S3 lacking binding activity was used at a concentration of 18.5 μg/ml. (C) Binding of the scFv antibody cloned from cell S9 to CD28-Fc. The graph depicts absorption values (AU) for the scFv antibody preparation in serial twofold dilutions with concentrations in a range from 101 μg/ml to 0.78 μg/ml (dark grey). As a control the preparation of the scFv antibody cloned from another cell S4 lacking binding activity was used at a concentration of 109 μg/ml (light grey). (D) Lack of binding of the scFv antibody cloned from cell S9 to CD40-Fc. The graph depicts absorption values (AU) for the scFv antibody preparation in serial twofold dilutions with concentrations in a range from 101 μg/ml to 0.78 μg/ml (dark grey). As a control the preparation of the scFv antibody cloned from another cell S4 lacking binding activity was used at a concentration of 109 μg/ml (light grey).

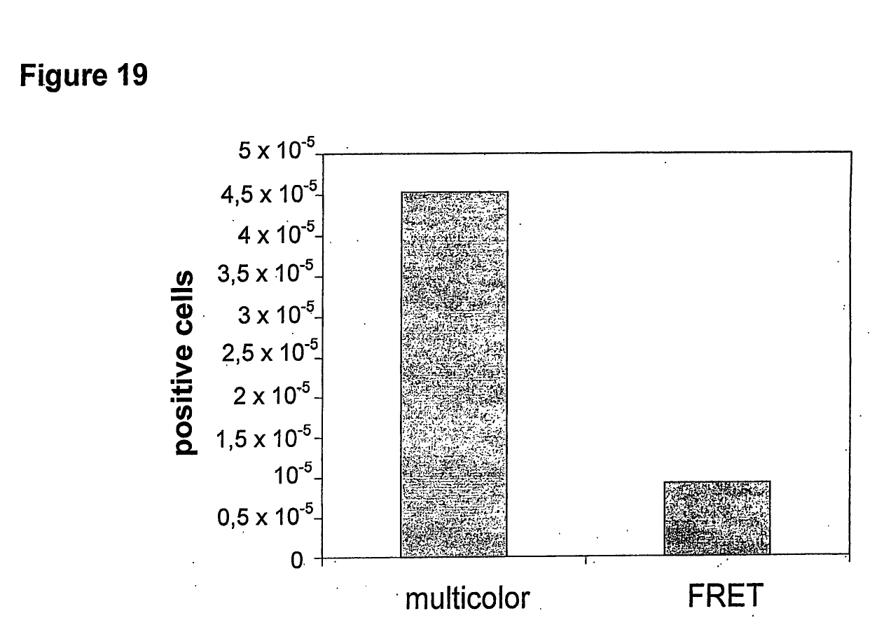

FIG. 19

Comparison of the FRET selection method with a multicolor FACS. The bar plot indicates the percentages of recombinant human CD28-mulg-specific naïve B cells selected by the FRET method (right bar) and by multicolor FACS (left bar).

The examples illustrate the invention.

### EXAMPLE 1

Selection of Single B Cells From a Mixture of Mouse B Cell Lines Using FRET

A) Description of Mouse B Cell Lines Used

Two mouse B cell lines were used to establish and determine the feasibility of FACS based B cell selection using fluorescence resonance energy transfer (FRET) as selection principle. Two mouse B cell lines with different antigen-specificity were chosen:
A20 cells: undetermined antigen-specificity: The A20 cell line is a BALB/c B cell lymphoma line derived from a spontaneous reticulum cell neoplasm found in an old BALB/cAnN mouse (Kim K J et al., 1979, J. Immunol. 122, 549-554).
The cells express little surface immunoglobulin when grown in Click's medium; however, they express large amounts when grown in RPMI 1640 medium.
The cells can present both alloantigens and protein antigens (Glimcher L H et al., 1982, J. Exp. Med. 155, 445-459),
8.18C5 cells with MOG-Fc antigen-specificity: Litzenburger et al., 1998, J. Exp. Med. 188(1), 169-180 generated a transgenic mouse strain with an anti-MOG heavy chain variable region, derived from the anti-MOG mAb 8.18-C5 “knocked in” for the germline J_{H} locus. Such mice exclusively express the 8.18-C5 anti-MOG heavy chain, resulting in generation of approximately 30% MOG-reactivity in the B-cell pool, as assessed by binding to recombinant MOG. Whole lymphocytes from transgenic knock-in mice were prepared from spleen.

Both B cell lines have surface IgG. Therefore, an anti mouse IgG-fluorescein conjugate is supposed to bind to both cell types. The fluorescein dye is the donor dye in the FRET assay. It appears in the FL1 channel of the FACS device. The MOG-Fc-Alexa Fluor 546 conjugate is supposed to accept the fluorescent energy transmitted by fluorescein. This red fluorescence appears in the FL2 channel of the FACS device. However, this energy transfer event only occurs when both dyes are in close proximity towards each other (within the “Foerster distance”). In case the MOG-Fc-Alexa Fluor 546 conjugate binds unspecifically to the surface of the B cell, there can be no signal due to the distance of donor and acceptor dye.

B) Mouse B Cell Staining

An amount of 200 000 cells from each B cell line was placed into the wells of a 96 well V-shape micro

liter plate and centrifuged at 600 g at 20° C. for 3 min. The supernatant was discarded and antibody solution added consisting of 50 μl FACS buffer plus 5 μl antibody (either anti mouse IgG-fluorescein or MOG-Fc-Alexa Fluor 546).

| Labeling | | | |
|---|---|---|---|
| reaction | Cell line | Anti Maus IgG Fluor. | MOG-Fc Alexa 564 |
| 1 | A20 | − | − |
| 2 | A20 | + | − |
| 3 | A20 | − | + |
| 4 | A20 | + | + |
| 5 | 8.18C5 | − | − |
| 6 | 8.18C5 | + | − |
| 7 | 8.18C5 | − | + |
| 8 | 8.18C5 | + | + |

The labeling reactions were incubated for 30 min at 4° C. Subsequently, the wells were filled up to 200 μl using FACS buffer. Cells were centrifuged as above, the supernatant was discarded. The washing procedure was repeated and cells resuspended in 200 μl FACS buffer containing 0.5 μg/ml propidium iodide as a death marker. Propidium iodide enters cells with membrane damage (dead cells) and marks them by binding to their DNA. The propidium iodide appears in the FL3 channel of the FACS device.

C) FACS Settings and Measurements

A FACS sorter (Becton Dickinson, US) was used with the following settings for 8.18C5 cells: FCS E00 1.0, SSC 396, FL1 468 log, FL2 489 log, FL3 495 log.

Labeling reaction 6 (see example 1B) containing only the donor-fluorochrome fluorescein displayed a fluorescent signal between 10² und 10³ in channel FL1. The compensation used for reaction 6 was FL2-FL1 24,6%, amplification was FL2: 490 (FIG. 2A). To see true FRET signal reaction 8 was measured. A strong shift in FL2 could be seen (FIG. 2B). Another labeling reaction was measured, reaction 7, to control for unspecific signal in FL2 (MOG-FC-Alexa Fluor 546 conjugate only), amplification FL2 490. With reaction 7 no FL2 shift could be observed (FIG. 2C). When the amplification of FL2 was increased to 500, the FRET signal was detected more clearly (FIG. 2D). As most crucial experiment reactions 4 and 8 were mixed to determine, if the two mouse B cell populations really could be separated by the FRET measurements (FIG. 2E). Both populations are detectable in FL1/FL2 as well as in FSC/SSC. According to the amount of 8.18C5 added the FRET gate appears fuller and fuller (compare FIG. 2F and 2E). As a direct negative control for the specificity of FRET selection in this mixing experiment reaction 4 was measured (only A20 cells stained). Thereby, no cells could be detected in the FRET gate (FIG. 2G).

As a result of these FACS staining experiments it could be shown that two mouse B cell lines could clearly be separated from each other depending on their antigen-specificity using FRET as the selection method. No FRET signal was generated in non antigen-specific, FRET-negative B cells (FIG. 2G). This is possible due to the specific characteristics of the fluorochromes chosen. Alexa Fluor 546 is not excitable at 488 nm. It needs the fluorescence resonance energy transfer from fluorescein.

### EXAMPLE 2

Verification of Single B Cell Identity By Nested PCR

A) A20- and MOG-primers Were Tested for Their Specificity.

RNA was isolated from 1.0×10⁷ cells each (Rneasy Mini Kit, Qiagen, Hilden, Germany). Complementary DNA was synthesized (Omniscript RT Kit, Qiagen, Hilden, Germany) using poly (dT)₁₅ primers (Roche, Penzberg, Germany). Subsequently to the reverse transcription reaction a nested PCR was performed using the following primer pairs:

| | | |
|---|---|---|
| 5′ β-actin mouse | ACC TTC AAC ACC | (SEQ ID NO.: 1) |
| | CCA GCC ATG | |
| 3′ β-actin mouse | GCT CGG TCA GGA | (SEQ ID NO.: 2) |
| | TCT TCA TGA GG | |
| 5′ VH-MOG-outside | GCT ACA CAT TCA | (SEQ ID NO.: 3) |
| | GTA GCT TC | |
| 3′ VH-MOG-outside | GTA TGG CAT GTT | (SEQ ID NO.: 4) |
| | TAC CAT CG | |
| 5′ VH-MOG-inside | TCA GTA GCT TCT | (SEQ ID NO.: 5) |
| | GGA TAG AG | |
| 3′ VH-MOG-inside | GTA TGG CAT GTT | (SEQ ID NO.: 6) |
| | TAC CAT CGT ATT | |
| | AC | |
| 5′ VH-A20-outside | GTT ACA ATT TCT | (SEQ ID NO.: 7) |
| | CCG ACA AG | |
| 3′ VH-A20-outside | GTC GCA GGC GGA | (SEQ ID NO.: 8) |
| | ATA ATC AC | |
| 5′ VH-A20-inside | TCT CCG ACA AGT | (SEQ ID NO.: 9) |
| | GGA TTC AC | |
| 3′ VH-A20-inside | GCA GGC GGA ATA | (SEQ ID NO.: 10) |
| | ATC ACC CG | |

Gene sequences of A20 were kindly provided by Dr. Ralph Mocikat, GSF München and Michael Hallek), MOG gene sequences provided by Marcel Zocher.

PCR was performed (30 cycles 0.5 min DNA synthesis each cycle and 55° C. annealing temperature; Robocycler^{R}, Stratagen, La Jolla, USA). Each PCR reaction contained 1 μl cDNA, 1 μl forward primer (from stock 10 μM), 1 μl reverse primer (from stock 10 μM), 2 μl dNTPs (from stock 2 mM each), 2 μl 10× TAQ Puffer (Sigma-Aldrich Chemie GmbH Munich, Germany), 0.2 μl TAQ-Polymerase (conc. 5 U/μl, Sigma-Aldrich Chemie GmbH Munich, Germany), 12.8 μl H₂O. The DNA amplification results were analysed on a standard analytical agarose gel. The result of this nested PCR was that the MOG primers amplified specifically just DNA from MOG cells, not from A20 cells and the A20 primers amplified specifically just DNA from A20 cells, not from MOG cells (FIG. 3A). As a positive control β-actin was amplified to test the quality of the CDNA and the PCR reactions.

B) RNA Isolation and Reverse Transcription from Single B Cells

Sorted mouse B cells from Example 1 were tested using MOG- and A20-specific nested PCR. A number of 8 sorted B cells was used for this test. Each of these 8 B cells was contained in 160 μl FACS buffer. The cells were lysed by adding 480 μl lysis-/binding buffer (Dynal Biotech GmbH, Hamburg, Germany, Dynabeads mRNA direct micro kit). At this point cells were stored at −20° C. As positive controls a pool of 200 A20 cells and another pool of 200 MOG cells were lysed in parallel with the sorted single cells.

In the next step the RNA of the lysed cells was coupled to magnetic beads (Dynal Biotech GmbH, Hamburg, Germany, Dynabeads mRNA direct micro kit). Before coupling beads were washed: 10×20 μl Dynabeads were washed in 200 μl lysis buffer. Beads were magnetically separated and the supernatant was discarded. This washing procedure was repeated two more times. Finally, beads were resuspended in 11 μl lysis buffer each. Subsequently 10 μl washed Dynabeads were added to each of the 10 RNA samples. The RNA and the beads were incubated under mixing for 10 min at room temperature. Subsequently, magnetic separation was performed, the supernatant was removed and the beads were washed two times with 100 μl washing buffer A (Dynal Biotech GmbH, Hamburg, Germany, Dynabeads mRNA direct micro kit) and one time with 100 μl WBI-buffer (50 mM Tris/HCL pH 8.0, 75 mM KCL, 10 mM DTT, 0.25% IGEPAL). After the last washing step the beads were resuspended with 7 μl H₂O for eluton of RNA from the beads.

The isolated RNA was subsequently subjected to a CDNA synthesis step. In contrast to the test experiment (see A above) a mixture of the outer primers was used for the CDNA synthesis from single cells: 1) 0.5 μl/sample 3′-VH A20-outside (10 μM stock); 2) 0.5 μl sample 3′-VH MOG-outside (10 μM stock). An amount of 1 μl primer mixture was added to each RNA probe. To allow primer annealing, samples were denatured for 3 min at 65° C. Samples were placed on ice for 5 min immediately after the annealing step. Reverse transcription was carried out using Sensiscript RT Kit, Qiagen, Hilden, Germany (2 μl 10× Sensiscript RT-buffer, 2 μl dNTPs 5 mM each, 1 μl Sensiscript-Reverse Transcriptase, 7 μl H₂O). Reverse transcription was performed at 37° C. for 60 min followed by a denaturation step at 95° C. for 5 min. Samples were stored on ice.

C) Nested PCR with Single Sorted Mouse B Cells

For DNA amplification the following nested PCR was carried out for each sample: First round of DNA amplification was performed using the outer primer pairs (cycler and program see above A but with 40 cycles instead of 30 cycles). Two reactions were performed for each sample. Reaction 1 contained the 5′VH MOG outside and the 3′VH MOG outside primer pair and a reaction 2 contained the 5′VH A20 outside and the 3′VH A20 outside primer pair.

A second round of PCR amplification was performed using the inner primer pairs (cycler and program see above A with 40 cycles instead of 30 cycles) and 3 μl of the first round PCR as DNA template. Again, for each sample two reactions were performed. Reaction 1 contained the 5′VH MOG inside and the 3′VH MOG inside primer pair and a second reaction contained the 5′VH A20 inside and the 3′VH A20 inside primer pair. The DNA amplification results were analysed on a standard analytical agarose gel. The result of this nested PCR amplification is seen in FIG. 3B and 3C. All eight cells were MOG positive as well as the MOG cell control containing the MOG 200 cells pool. As expected, the A20 200 cells pool didn't show any signal in the MOG nested PCR neither did the water control. The results of the A20 nested PCR showed no specific PCR amplified signal in 7 out of the 8 single sorted cells tested. The sample containing cell number 3 showed MOG as well as A20 PCR amplified signal. It is likely that sample number three actually contained 2 cells, one MOG cell and a contaminating A20 cell. This might well be the result of manual sample collection.

### EXAMPLE 3

Specificity of FRET Selection, Dilution Series Experiments

This experiment employs a dilution series of IgG positive, MOG-Fc specific 8.18C5 mouse B cells in IgG positive but antigen unspecific A20 mouse B cells to determine the specificity of the FRET selection method. First the FRET gate was set using a double stained 8.18C5 B cell population. Thereafter, dilutions of double stained 8.18C5 B cells in A20 cells were measured. The ratio of double stained 8.18C5 B cells used and cells measured in the FRET gate reflects the specificity of the FRET method. Additional, A20 cells were used to control the. FRET gate. Labeling reactions were performed in a 96 well plate format. A number of 200 000 cells was added to each well. Reactions 1-7 were used as controls to determine the FACS gate settings. Labeling reaction 2 for unstained A20 cells contained 48.5 μl A20 cells (fresh from cell culture, 4.12×10⁶/ml) and 200 μl FACS buffer (1% BSA, 0.05% sodium azid), set up four times. Labeling reaction 2 for unstained 8.18C5 cells contained 83 μl 8.18C5 cells (fresh from cell culture, 2.4×10⁶/ml) and 50 μl FACS buffer. Labeling reaction 3 for IgG stained A20 cells as control of overshining (first compensation Fl2-F1) contained 48.5 μl A20 cells and 200 μl FACS buffer and 10 μl goat anti mouse IgG-fluorescein 0.48 mg/ml (200 μl polyclonal goat anti mouse IgG 1 mg/ml, Dianova, Hamburg, Germany+10 μl fluorescein-NHS 1.3 mg/ml, Fluka, Riedel-de Haen, Sigma-Aldrich, Seelze, Germany, incubated for 1 hr at room temperature (Micromet Lot 12.07.01, Munich)). Labeling reaction 4 for double stained A20 cells as control for unspecific MOG-binding contained 48.5 μl A20 cells and 250 μl FACS buffer and 25 μl goat anti mouse IgG-fluorescein and 25 μl MOG-Fc Alexa Fluor 564, 0.527 mg/ml (100 μl MOG-Fc (prepared as described in Marcel Zocher, PhD thesis)+5 μl Alexa 546-NHS, 1.5 mg/ml, Molecular Probes, Eugene, Oreg., USA, incubated for 1 hr at room temperature (Micromet Lot PH2024, Munich)). Labeling reaction 5 for MOG-Fc single stained 8.18C5 cells as control for FL2 by residual excitement of Alexa Fluor 546 contained 83 μl 8.18C5 cells and 50 μl FACS-Puffer and 5 μl MOG-Fc Alexa Fluor 546. Labeling reaction 6 for IgG single stained 8.18C5 cells with setting of the amplification (FL2-FL1/FL3-FL2) contained 83 μl 8.18C5 cells and 100 μl FACS-Puffer and 10 μl goat anti mouse IgG-fluorescein. Labeling reaction 7 for double stained 8.18C5 cells with settings for the FRET region contained 83 μl 8.18C5 cells and 200 μl FACS-Puffer and 20 μl goat anti mouse IgG-fluorescein and 20 μl MOG-Fc Alexa Fluor 546.

Reactions 8-18 were set up as dilution series (see table). All reactions contained 48.5 μl (=200 000) A20 cells (fresh from cell culture, 4.12×10⁶/ml), 20 μl goat anti mouse IgG-fluorescein and 20 μl MOG-Fc Alexa Fluor 546. Additionally, the reactions contained decreasing numbers of 8.18C5 cells (from 200 000 cells in 1:2 dilution steps down to 195 cells). Therefore, 83 μl, 41.5 μl, 20.75 μl, 10.4 μl, 5.2 μl, and 2.6 μl of 8.18C5 cells (fresh from cell culture, 2.4×10⁶/ml) were added to reactions 8-13 respectively. Further, 13 μl, 6.5 μl, 3.24 μl of 1:10 diluted 8.18C5 cells were added to reactions 14 to 16, respectively. Finally, 16.2 μl and 8.1 μl of 1:100 diluted 8.18C5 cells were added to reactions 17-18, respectively.

The FACS control reactions 5, 6 and 7 are shown in FIG. 4A, B and C. The separation of the double stained A20 and the 8.18C5 B cells is shown in FIG. 4D. FIG. 4E shows the same separation of double stained A20 and 8.18C5 B cells as FIG. 4D. FIG. 4E served as positive control for FRET gate setting: gate RI on living FL1 positive cells and gate R2 on FRET positive living cells. The graph of all dilution reactions is shown in FIG. 5. The table below summarises the results of the dilution experiment. The values of the strongest dilutions (reactions 14-18) deviate somewhat from the expected values due to low IgG signal on the 8.18C5 cells.

| | | | | ² % | ³ cells | ⁴ sorted |
|---|---|---|---|---|---|---|
| | cells | | | 8.18C5 | 8.18C5 | 8.18C5 |
| reaction | in R1 | ¹ A20 | 8.18C5 | expected | expected | cells in R2 |
| 8 | 5245 | 200.000 | 200.000 | 50.0 | 2623 | 2858 |
| 9 | 5188 | 200.000 | 100.000 | 33.3 | 1729 | 1755 |
| 10 | 5183 | 200.000 | 50.000 | 20.0 | 1037 | 1111 |
| 11 | 5180 | 200.000 | 25.000 | 11.1 | 576 | 760 |
| 12 | 5183 | 200.000 | 12.500 | 5.9 | 305 | 512 |
| 13 | 5183 | 200.000 | 6.250 | 3.0 | 157 | 128 |
| 14 | 5183 | 200.000 | 3.125 | 1.5 | 80 | 47 |
| 15 | 5180 | 200.000 | 1.563 | 0.8 | 40 | 43 |
| 16 | 5198 | 200.000 | 781 | 0.4 | 20 | 44 |
| 17 | 5161 | 200,000 | 391 | 0.2 | 10 | 29 |
| 18 | 5203 | 200.000 | 195 | 0.1 | 5 | 22 |

¹ Number of cells mixed;
² Theoretical percentage of 8.18C5 cells;
³ Theoretical number of sorted 8.18C5 cells;
⁴ Number of actually sorted 8.18C5 cells measured within the FRET gate R2.
The dilution experiment as performed here demonstrates the high specificity of the FRET selection method.

### EXAMPLE 4

Selection of Single B Cells from Human Blood Using FRET

A) Isolation of PBMCs

To isolate human peripheral blood mononuclear cells (PBMCs) 500 ml heparinized blood were collected from a healthy donor. The blood was diluted 1:1 with PBS (Phosphate Bufferd Saline). The resupended blood cells were separated using a Ficoll density gradient (Ficoll-Paque from Amersham Biosciences Europe GmbH, Freiburg, Germany, density 1.077 g/ml). A centrifuge tube was filled with 15 ml Ficoll solution and gently overlaid with 30 ml of the blood/PBS mixture. The gradient was centrifuged at 600 gₐᵥ for 30 min at 20 ° C. PBMCs were removed from the gradient and transferred to a fresh tube. A volume of 45 ml FACS-buffer (1% FCS in PBS) was added to the PBMCs to wash the cells. Cells were spun down at 600 gₐᵥ for 10 min, the supernatant discarded and cells resuspended in further 15 ml of FACS-buffer (1% FCS in PBS, no azid). PBMCs were counted using a Neubauer chamber.

B cells were isolated from PBMCs using Miltenyi purification. The B Cell Isolation Kit is an indirect magnetic labeling system which is used to obtain untouched B cells from peripheral blood by the magnetic depletion of T cells, NK cells, monocytes, granulocytes, platelets and erythroid precursor cells. A cocktail of hapten-modified CD2, anti-IgE, CD4, CD11b, CD16 and CD36 antibodies is used for labeling non-B cells. In a second step, non-B cells are magnetically labeled using MACS MicroBeads coupled to an anti-hapten antibody (Bauer et al., 1999, Immunol. 97, 699-705). The protocol was performed as described (Milteny B cell isolation kit, Milteny, Auburn, Calif.). Cells were counted and resuspended in 10% FCS/PBS no azid (MACS buffer) at a concentration of 4.25×10⁶ cells/ml.

B) Labeling of The Detection molecules rCD28 Antigen and Anti Human IgD Antibody

Before the actual labeling of cells the fluorophores fluorescein and Alexa Fluor 546 were attached (coupled) to polyclonal rabbit anti human IgD antibody, preferably 1 mg/ml in TRIS-buffer, (DAKO, Hamburg, Germany) and recombinant human CD28-murine Ig (recCD28-mulg/rCD28), preferably 0.5 mg/ml in phosphat/potassium-buffer, (Ancell Corp., Bayport, USA) respectively. Human CD28 is an important costimulatory molecule found on all CD4+ T cells and on about half of the CD8+T cells. T cell activities attributed to CD28 include prevention of anergy, induction of cytokine gene transcription, stabilization of cytokine mRNAs and activation of CD8+ cytotoxic T lymphocytes. rCD28 is a soluble fusion protein consisting of the extracellular (134 aa) domain of human CD28 fused to murine IgG2a Fc (232 aa). Anti human IgD antibody and rCD28 antigen were dialyzed against borate buffer pH 8.5 (0.1 M NaCl, 0.05 M Borate, H2O Ampuwa) for 3×1 h in dialysis tubing (Visking, MWCO 10.000, Roth, Karlsruhe, Germany). The protein amount after dialysis was measured (Bradford Reagent, Bio-Rad Laboratories Inc., Hempstead, UK) using a bovine IgG protein standard 2 mg/ml in PBS (Pierce 23212, Pierce Biotechnology, Rockford, Ill., USA) and anti human IgD antibody and rCD28 antigen concentrations were calculated. The concentrations of anti human IgD antibody and rCD28 after dialysis was 1.445 mg/ml and 1.064 mg/ml, respectively.

The fluorochromes fluorescein-NHS, preferably 1.1 mg/ml in DMSO (Fluka, Riedel-de Haen, Sigma-Aldrich, Seelze, Germany) and Alexa Fluor 546 NHS, preferably 5 mg/ml in DMSO (Molecular Probes, Eugene, Oreg., USA) were subsequently conjugated to anti human IgD antibody and rCD28, respectively. For each molecule two conjugation reactions were carried out: one having a 10fold molar excess of the fluorochrome and the other one having a 5fold molar excess of the fluorochrome.

In a first reaction, i.e. for coupling with 10fold molar excess, 100 μl anti human IgD antibody and 5 μl fluorescein-NHS, 1.1 mg/ml in DMSO were mixed and incubated for 1 h on the Vortex mixer (IKA Technologies, Germany). In a second reaction, i.e. for coupling with 5fold molar excess, 100 μl anti human IgD antibody and 2.5 μl fluorescein-NHS, 1.1 mg/ml in DMSO and 2.5 μl DMSO (HPLC grade, Sigma-Aldrich, Seelze, Germany) were incubated for 1 h on the Vortex mixer. In a third reaction 100 μl rCD28 and 1.44 μl Alexa Fluor 546 NHS, 5 mg/ml in DMSO (Molecular Probes, Eugene, Oreg., USA) and 3.56 μl DMSO were incubated for 1 h on the Vortex (IKA Technologies, Germany).

In a fourth reaction 100 μl rCD28 and 0.72 μl Alexa Fluor 546 NHS, 5 mg/ml, in DMSO (Molecular Probes, Eugene, Oreg., USA) and 4.28 μl DMSO were incubated for 1 h on the Vortex (IKA Technologies, Germany).

The conjugates were purified using 2 ml P60 gel each equilibrated with PBS, 0.05% sodium azid. The product obtained from reaction 1, i.e. the anti human IgD antibody coupled to fluorescein-NHS by 10 fold molar excess, was used for B cell selection.

C) Labeling of the B Cells

The isolated cells from A) were divided up into four small labeling reactions used as controls and into one big labeling reaction used for the sort.

The first labeling reaction contained unstained cells. Therefore, 400 000 cells were diluted into 100 μl FACS buffer (=1% heat-inactivated FCS in PBS without calcium and magnesium, pH 7.4).

The second labeling reaction contained single stained cells with the green fluorescence donor fluorescein. Therefore, 400 000 cells were diluted into 100 μl FACS buffer and 10 μl of rabbit anti human IgD-fluorescein (Micromet Lot. PH2006, Munich) were added.

The third labeling reaction contained a control for auto-fluorescence of the acceptor fluorochrome Alexa Fluor 546 (25% anti human IgD-Alexa Fluor 546 and 75% non fluorescently marked rabbit anti human IgD antibody). Therefore, 2.5 μl of rabbit anti human IgD antibody, preferably 1 mg/ml in borate buffer pH 8.5, (DAKO, Hamburg, Germany) and 2.5 μl of rabbit anti human IgD-Alexa Fluor 546 (ca. 0.3 mg/ml, 200 μl anti human IgD, 1 mg/ml, DAKO, Hamburg, Germany+5 μl Alexa 546-NHS, 1.5 mg/ml, Molecular Probes, Eugene, Oreg., USA, incubated for 1 hr at room temperature (Micromet Lot. PH2006, Munich)) were mixed and then added to 400 000 cells diluted in 100 μl FACS buffer.

The fourth labeling reaction contained an IgD double staining as positive control and guidance for the gate setting. Therefore, 15 μl of rabbit anti human IgD-fluorescein (Micromet Lot. PH2006, Munich) and 5 μl of rabbit anti human IgD-Alexa Fluor 546, (ca. 0.3 mg/ml) (Micromet Lot. PH2006, Munich, obtained as described above) were mixed and then added to 400 000 cells diluted in 100 μl FACS buffer (1% heat-inactivated FCS in PBS without calcium or magnesium, pH 7.4).

A large labeling reaction used for actual sorting contained all remaining cells diluted in 15 ml FACS buffer. Therefore, 100 μl of rabbit anti human IgD-fluorescein (0.3 mg/ml) (Micromet Lot. PH2006, Munich) and 100 μl of rCD28-Alexa Fluor 546 (0.21 mg/ml in PBS containing 0.05% sodium azid) (Micromet Lot PH2006, Munich) were mixed and added to the cells.

The labeling reactions was incubated for 30 min at 4° C., then washed twice with FACS buffer. Each of the four control reactions was resuspended in 400 μl FACS buffer containing 0.5 μg/ml propidium iodide as a death marker. The sorting reaction was resuspended in 400 μl FACS buffer containing 0.5 μg/ml propidium iodide as a death marker.

D) FACS Sort Preparations

Before FACS sorting was started the FACS-flow containers were rinsed with PBS pH 7.4 diluted from stock with Ampuwa H₂O. Subsequently, the FACS-flow container was filled with PBS containing no azid and the probe collection tube filled with Ampuwa H₂O was placed at the collection position. The control panels of the FACS liquid system were set to run and the acquisition control was set to acquire. The whole system was washed for 5 min. After that the machinery was kept at standby.

E) FACS Settings

The labeling reactions from C) were used to adjust FACS settings, select compensation and finally choose appropriate settings. This was achieved by performing several measurement steps. For the first labeling reaction (unstained cells) the compensation was set to 0, FL1-FL3 to 10⁰-10¹. For the second labeling reaction (single stain with anti IgD-fluorescein) the compensation was set to FL2-FL1 ca. 25%, compensation for FL3-FL2 ca. 4%. For the third labeling reaction (25% anti IgD-Alexa546 and 75% non-fluorescent anti-IgD) the gridlock setting was set to highest FL2. For the fourth labeling reaction (anti IgD-fluorescein and anti IgD-Alexa546) the Gate settings for fluorescence resonance energy transfer (FRET) were set above Alexa Fluor 546 auto-fluorescence. For the large labeling reaction for FACS sort (anti IgD-fluorescein and rCD28-Alexa Fluor 546) the gate settings were the same as from labeling reaction four.

The gate for selection of living cells represented a combination of three criteria. On one hand the gate restricted the selected cells to the FSC/SSC living population (low granularity) on the other hand only FL1 positive cells (=IgD+) were selected and as third criterium only FL3 negative cells (no propidium iodide staining) were selected.

F) FACS Sort Procedure

Just before the actual sorting process, the FACS sort machine was washed again with PBS at high flow rate. Immediately after this, the actual flow sort was initiated by switching to a low flow rate of 12 μl/sec. This flow rate equals an analysis of 1500 cells/sec. Cells were diluted 1:2 in MACs-buffer just before sorting. For sorting results see FIG. 6A-D. FIG. 6A showed FACS signals of unstained cell. FIG. 6B showed the background caused by auto-fluorescence. FIG. 6C showed the background of Alexa Fluor 546 acceptor dye staining. The double stained cells from labeling reaction four (see example 4C) were used as guidance to set the gate for selection (FIG. 6D).

Autoreactive B cells having bound rCD28-Alexa Fluor 546 antigen and anti IgD-fluorescein simultaneously could be identified using FACS sort due to the high stringency of fluorescence resonance energy transfer (FRET) signals. The sort results showed that a minute number of cells was selected as FRET-positive (FIG. 6E). For further validation of these selected B cells single clones were analyzed using VH and VL PCR cloning.

### EXAMPLE 5

Cloning of VH and VL Regions from Isolated B-cells

A) RNA Isolation and cDNA Synthesis

The VH and VL antibody chains were cloned from several isolated cells. The single B cells were collected in a volume of 160 μl FACS buffer, lysed with 480 μl lysis/binding buffer (Dynal Biotechnology GmbH, Hamburg, Germany) and stored at −20 ° C. The washing of the Dynabeads and the RNA extraction procedure was performed as described above (example 2B). For cDNA synthesis a primer mix was generated containing four different 3′-primers. Each primer binds to the constant region:

| | | |
|---|---|---|
| M For 1: | TGG CAG ATG AGC | (SEQ ID NO.: 11) |
| | TTG GAC TTG | |
| K For: | ACA CTC TCC CCT | (SEQ ID NO.: 12) |
| | GTT GAA GCT | |
| L For: | GTG CTC CCT TCA | (SEQ ID NO.: 13) |
| | TGC GTG AC | |
| Hu, β-actin For 3: | ACT CGT CAT ACT | (SEQ ID NO.: 14) |
| | CCT GCT TGC | |

Reactions contained 0.5 μl/Probe heavy chain primer M For 1 (10 μM stock), 0.5 μl/Probe light chain kappa primer K For (10 μM stock), 0.5 μl/Probe light chain lambda primer L For (10 μM stock), 0.5 μl/Probe β-actin primer hu. β-actin For 3 (10 μM stock). Annealing of 2 μl primer mix to each sample was performed at 65° C. for 3 min. Samples were placed on ice for 5 min immediately after the annealing step. Reverse transcription was carried out using Sensiscript RT Kit (Qiagen, Hilden, Germany) (2 μl 10× Sensiscript RT-buffer, 2 μl dNTPs 5 mM each, 1 μl Sensiscript-Reverse Transcriptase, 6 μl H₂O). Reverse transcription was performed at 37° C. for 60 min followed by a denaturation step at 95° C. for 5 min. Samples were stored on ice.

B) Half Nested PCR Amplification of VH and VL Regions

In the half nested PCR two rounds of DNA amplification are performed as with nested PCR. However, during half nested PCR the 5′ primers stayed constant for both PCR amplification steps and only the 3′ primers were shifted inside during the second round of PCR.

For the first round PCR the following primers were used:

| | | | | |
|---|---|---|---|---|
| M1 | heavy chain | Primer: hu CH 1 M For | + | hu VH back MIX |
| K1 | light chain | Primer: K For 2 | + | hu VK back MIX |
| | kappa | | | |
| L1 | light chain | Primer: L For 2 | + | hu VI back MIX |
| | lambda | | | |
| B1 | β-actin chain | Primer: hu β-actin For 2 | + | hu β-actin back1 |

| | | |
|---|---|---|
| HUCH1MFOR: | TGG AAG AGG CAC GTT | (SEQ ID NO.: 15) |
| | CTT TTC TTT | |
| KFOR2: | AGT TAC CCG ATT GGA | (SEQ ID NO.: 16) |
| | GGG CG | |
| LFOR2: | CCT TCC AGG CCA CTG | (SEQ ID NO.: 17) |
| | TCA C | |
| HUBACTINBACK1: | GTG GGG CGC CCC AGG | (SEQ ID NO.: 18) |
| | CAC CA | |
| HUBACTINFOR2: | GAT GGA GGC GGC GAT | (SEQ ID NO.: 19) |
| | CCA CAC GG | |

hu VH back MIX:

| | | |
|---|---|---|
| HUVHBACK1: | CAG RTG CAG CTG GTG | (SEQ ID NO.: 20) |
| | CAR TCT GG | |
| HUVHBACK2: | SAG GTC CAG CTG GTR | (SEQ ID NO.: 21) |
| | CAG TCT GG | |
| HUVHBACK3: | CAG GTC CAG CTT GTA | (SEQ ID NO.: 22) |
| | CAG TCT GG | |
| HUVHBACK4: | SAG RTC ACC TTG AAG | (SEQ ID NO.: 23) |
| | GAG TCT GG | |
| HUVHBACK5: | SAG GTG CAG CTG GTG | (SEQ ID NO.: 24) |
| | GAR TCT GG | |
| HUVHBACK6: | GAG GTG CAG CTG KTG | (SEQ ID NO.: 25) |
| | GAG WCY GG | |
| HUVHBACK7: | CAG CTG CAG CTA CAG | (SEQ ID NO.: 26) |
| | CAG TGG GG | |
| HUVHBACK8: | CAG STG CAG CTG CAG | (SEQ ID NO.: 27) |
| | GAG TCS GG | |
| HUVHBACK9: | GAR GTG CAG CTG GTG | (SEQ ID NO.: 28) |
| | CAG TCT GG | |
| HUVHBACK10: | CAG GTA CAG CTG CAG | (SEQ ID NO.: 29) |
| | CAG TCA GG | |

hu VK back MIX:

| | | |
|---|---|---|
| HUVκBACK1: | GAC ATC CRG DTG ACC | (SEQ ID NO.: 30) |
| | CAG TCT CC | |
| HUVκBACK2: | GAA ATT GTR WTG ACR | (SEQ ID NO.: 31) |
| | CAG TCT CC | |
| HUVκBACK3: | GAT ATT GTG MTG ACB | (SEQ ID NO.: 32) |
| | CAG WCT CC | |
| HUVκBACK4: | GAA ACG ACA CTC ACG | (SEQ ID NO.: 33) |
| | CAG TCT CC | |
| HUVκBACK5: | GAT GTT GTG ATG ACT | (SEQ ID NO.: 34) |
| | CAG TCT CC | |
| HUVκBACK6: | GAT ATT GTG ATG ACC | (SEQ ID NO.: 35) |
| | CAC ACT CC | |
| HUVκBACK7: | GAA ATT GTG CTG ACT | (SEQ ID NO.: 36) |
| | CAG TCT CC | |

hu VL back MIX:

| | | |
|---|---|---|
| HUVλ BACK1: | CAG TCT GTS BTG ACG | (SEQ ID NO.: 37) |
| | CAG CCG CC | |
| HUVλ BACK2: | TCC TAT GWG CTG ACW | (SEQ ID NO.: 38) |
| | CAG CCA C | |
| HUVλ BACK3: | TCC TAT GAG CTG AYR | (SEQ ID NO.: 39) |
| | CAG CYA CC | |
| HUVλ BACK4: | CAG CCT GTG CTG ACT | (SEQ ID NO.: 40) |
| | CAR YC | |
| HUVλ BACK5: | CAG DCT GTG GTG ACY | (SEQ ID NO.: 41) |
| | CAG GAG CC | |
| HUVλ BACK6: | CAG CCW GKG CTG ACT | (SEQ ID NO.: 42) |
| | CAG CCM CC | |
| HUVλ BACK7: | TCC TCT GAG CTG AST | (SEQ ID NO.: 43) |
| | CAG GAS CC | |
| HUVλ BACK8: | CAG TCT GYY CTG AYT | (SEQ ID NO.: 44) |
| | CAG CCT | |
| HUVλ BACK9: | AAT TTT ATG CTG ACT | (SEQ ID NO.: 45) |
| | CAG CCC C | |
| HUVλ BACK10: | CAG TCT GTG CTG ACT | (SEQ ID NO.: 46) |
| | CAG CCA CC | |
| HUVλ BACK11: | CAA TCT GCC CTG ACT | (SEQ ID NO.: 47) |
| | CAG CCT | |
| HUVλ BACK12: | TCT TCT GAG CTG ACT | (SEQ ID NO.: 48) |
| | CAG GAC CC | |
| HUVλ BACK13: | CAC GTT ATA CTG ACT | (SEQ ID NO.: 49) |
| | CAA CCG CC | |
| HUVλ BACK14: | CAG GCT GTG CTG ACT | (SEQ ID NO.: 50) |
| | CAG CCG TC | |
| HUVλ BACK15: | CWG CCT GTG CTG ACT | (SEQ ID NO.: 51) |
| | CAG CCM CC | |

Sequence symbols are: A (adenin), C (cytosine), G (guanosin), T (thymidin), I (inosin), U (uracil). Wobble IUPAC-IUB symbols are: R (A or G), Y (C or T), M (A or C), K(G or T), S (G or C), W (A or T), H (A or C or T), B (G or T or C), V (G or C or A), D (G or T or A), N (G or A or T or C).

Primers were slightly modified according to de Haard et al, 1999, JBC 274, 18218-18230; Sblattero and Bradbury, 1998, Immunotechnology 3, 271-278.

Subsequent PCR was performed (HotStar TAQ, QIAGEN, Hilden, Germany) on a RoboCycler^{R}(Stratagen, La Jolla, USA) using 40 cycles of DNA synthesis 0.5 min each cycle at 55° C. annealing temperature.

A second round of PCR amplification was performed using the following primers and 3μl of the first round PCR as DNA template (cycler and program as above):

| | | | | |
|---|---|---|---|---|
| M2 | heavy chain | Primer: IgM For | + | hu VH back MIX |
| K2 | light chain | Primer: IgK For | + | hu VK back MIX |
| | lambda | | | |
| L2 | light chain | Primer: IgL For | + | hu VL back MIX |
| | kappa | | | |
| B2 | β-actin chain | Primer: hu β-actin For1 | + | hu β-actin back1 |

| | | |
|---|---|---|
| IGMFOR: | GGT TGG GGC GGA TGC | (SEQ ID NO.: 52) |
| | ACT CC | |
| IGKFOR: | GAT GGT GCA GCC ACA | (SEQ ID NO.: 53) |
| | GTT CG | |
| IGLFOR: | GGA GGG YGG GAA CAG | (SEQ ID NO.: 54) |
| | AGT GAC | |
| HUBACTINFOR1: | CTC CTT AAT GTC ACG | (SEQ ID NO.: 55) |
| | CAC GAT TTC | |

The DNA amplification results were analysed on a standard 1.5% agarose gel. The half nested PCR results in several of the cells tested showed amplification of both V chains, VH and VL. A few cells immediately tested positive for VH and VL.

C) Subcloning of VH and VL Regions

The bands of VH and VL amplification fragments from example 5B were excised and isolated from the agarose gel. The isolated DNA fragments were subcloned into pCR2.1-TOPO (Invitrogen GmbH, Karlsruhe, Germany), clones were picked, plasmid DNA isolated and sequenced.

For each single cell one VH and one VL sequence was selected, which displayed the complete sequence including leader sequence and all functional sequence, the CDRs 1-3 and corresponding framework, which had no stop-codon mutation, nor frame shift and were clearly germ line sequences.

D) Fusion PCR for Generation of scFvs from Different VH and VL Regions

The cloned VH and VL sequences were fused together employing a fusion PCR technique. For selected clones linker primers were designed containing specific V sequence from the clone and additional linker sequence. VL and VH were fused together in the order of VL-VH, whereby the first linker primer was a 3′ linker for the VL clone plus linker sequence and the second linker primer was a 5′ linker for the VH clone plus linker sequence. The following principle sequence was used for the fusion primers:

| | | |
|---|---|---|
| VL 3′ linker primer: | GGA GCC GCC | (SEQ ID NO.: 56) |
| | GCC GCC AGA | |
| | ACC ACC ACC | |
| | ACC (X)ₙ | |
| VH 5′ linker primer: | TCT GGC GGC | (SEQ ID NO.: 57) |
| | GGC GGC TCC | |
| | GGT GGT GGT | |
| | GGT TCT | |
| | (X)ₙ | |

(X)ₙ denotes a variable number of nucleotides which are part of the sequence of specific VL or VH clones. The length of VL or VH specific sequence incorporated within these fusion primers depends upon the GC content of the sequence. The length of the matching sequence was optimized according to standard protocols for a melting temperature, which is favourable for PCR amplification. Preferably, the primer design allows to achieve an approximate overall oligonucleotide melting temperature of 68° C.

A first PCR amplification was performed using a VL sequence specific 5′ forward primer and the VL 3′ linker primer (RoboCycler^{R} Stratagene, La Jolla, USA, 30 cycles 1 min DNA synthesis and 55° C. annealing temperature). A second PCR amplification was performed under the same conditions using the VH 5′ linker primer and a VH sequence specific 3′ primer. The specific 5′ forward and 5′ linker primers were chosen according to the published germline sequences to remove primer derived deviations from the germline sequence generated in the initial amplification of the V-region sequences with degenerated 5′ primers.

The amplified PCR products were purified on a 1.5% agarose gel and, subsequently, the VL and VH specific bands were cut out and isolated from the gel (Qiaex kit, Qiagen, Hilden, Germany). Each DNA was resuspended in 50 μl H₂O. Thereof, 3 μl were used for further fusion PCR amplification. In the fusion PCR amplification the previously amplified and isolated VL and VH chains were mixed together and amplified using the outer VL and VH specific primers already used in the first amplifications (3 μl of each V chain template DNA, 3 μl of each primer, 6 μl dNTPs (10 μM stock), 6 μl 10× buffer from Sigma-Aldrich, 0.6 μl Taq polymerase from Qiagen, 35.4 μl H₂O, RoboCycler^{R} Stratagene, La Jolla, USA, 10 cycles with 1.5 min DNA synthesis at 55° C. annealing temperature). Due to the overlap in the linker sequences one continuous VL-linker-VH fusion product was amplified. This PCR fusion product was purified on a 1.5% agarose gel and isolated as described above. The resuspended isolation products were cut enzymatically to create the appropriate 5′ and 3′ overhangs for subcloning into a vector for example Bluescript (Sambrook & Russel: Molecular Cloning: A Laboratory Manual, third edition 2001, Cold Spring Harbor Laboratory Press, Cold Spring Harbor, N.Y.). The created plasmid was transformed into competent cells for example XL-1-blue cells (Stratagene, La Jolla, USA). Several of those transformed clones were picked, cultivated, plasmid DNA isolated from those cultivated cells and their identity verified by means of analytical enzymatical digest and sequencing or the like.

E) Expression of Cloned scFvs and Binding Assays

Sequence verified scFv clones were used for further subcloning into an expression vector system like pEF DHFR (InvitrogenGmbH, Karlsruhe, Germany). The scFv clones created had the structure: Leader- VL- (G₄S)₃- VH-Flag. Other structure orientations may be achieved by using a different fusion strategy. Transfected CHO cells transiently expressed the scFv constructs using standard protocols (Sambrook & Russel: Molecular Cloning: A Laboratory Manual, third edition 2001, Cold Spring Harbor Laboratory Press, Cold Spring Harbor, N.Y.). For further preservation of the clones, stable expressing CHO transfectants were selected for each scFv also according to the state of the art.

To confirm the specificity of the scFv clone the culture supernatant of scFv expressing CHO cells was used in an ELISA binding assay.

The isolated and verified VH and VL sequences could further be used to generate a variety of antibody constructs comprising single chain antibodies, bispecific antibody constructs and complete immunoglobulin formats.

### REFERENCE EXAMPLE 1

Multicolor Sort of Human B Cells for EpCAM Binders

A) Isolation of CD19⁺ Cells

Peripheral human blood mononuclear cells (PBMCs) were isolated using a Ficoll density gradient (Ficoll-Paque, Amersham Biosciences Europe GmbH, Freiburg, Germany, density 1,077 g/ml) according to manufacturer's protocol. Subsequently, 200×10⁶ cells were incubated with 100 μl CD19 beads for 15 min at 4° C. to isolate CD19⁺ cells. After washing and filtering 6.9×10⁶ cells were counted.

B) Labeling and FACS Detection of Cells

The Isolated cells were divided up into seven reaction tubes (˜1×10⁶ cells per 1 ml) and incubated with different amounts of Cy2-labeled EpCAM antigen (5.00 μg/ml, 2.50 μg/ml, 1.25 μg/ml, 0.63 μg/ml, 0.31 μg/ml). Another reaction contained unstained cells. A control reaction tube contained Cy2 labeled anti-CD45. All tubes were incubated at 4° C. for 30 min. Cells then were incubated with a goat anti human IgD polyclonal antiserum, which had been labeled with phycoerythrin (PE). Subsequently performed FACS sorting results are shown in FIG. 7A-F. In comparison to the anti-CD45 control (FIG. 7F), which showed a fluorescent shift (FL1-H) of nearly all B cells, the EpCAM stained cells showed hardly any shifted cells (FIG. 7A-E). Nevertheless, single cells with high fluorescent signals on both channels (FL2-H and FL1-H) were sorted by FACS.

C) Binding Assay with EpCAM Isolated scFvs

The RNA from single FACS sorted cells was isolated and the VH and VL regions were cloned via RT-PCR as described in example 2B and 5. After subcloning, supernatant from three subclones scEpCAM20-5, scEpCAM20-6 and scEpCAM20-7 were tested in FACS-based binding assays using KATOIII cells and CHO 17-1A cells (FIG. 8) as EpCAM positive cell lines. In both assays, bispecific scFv anti-EpCAM×anti-CD3, a known single chain antibody having anti-EpCAM specificity was used as positive control (FIG. 8 green line). Anti-His tag antibody and anti-EGFR antibody were used as negative control (FIG. 8 black line and pink line respectively). While bispecific scFv anti-EpCAM×anti-CD3 showed binding in both assays, none of the other substances showed any EpCAM binding activity. The multicolor sort of human B cells for EpCAM binders using Cy2 (similar to fluorescein) and phycoerythrin (PE) fluorescent labels resulted in no specific binders. Several reasons attributed to this result. Fluorescein works as a donor dye for phycoerythrin (PE). PE is a big protein molecule that has a molecular weight of 240 kD and its spectrum largely overlaps with fluorescein (FIG. 9). So in the described method, an IgD bound antigen would be surrounded by several polyclonal anti IgD-PE conjugates. This would cause a partial decrease of fluorescein signal due to PE size and spectrum overlap, since both fluorochromes are very close together. Furthermore, the PE conjugates like the one of the rabbit anti-fluorescein/Oregon Green IgG antibody (A-21250, Anti-Fluorescein/Oregon Green Antibodies and Conjugates) have the unique characteristics of both shifting the green-fluorescence emission of fluorescein-labeled probes to longer wavelengths and greatly intensifying the long-wavelength signal (www.probes.com/handbook/sections/0704.html). Additionally, it has been reported (Szaba et al., 1992, Biophys. J. 61(3), 661-70) that the energy transfer efficiency was down to approximately 20% between the CD4 epitopes OKT4-FITC and Leu-3a-PE as well as between OKT4E-FITC and OKT4-PE due to photobleaching. Between OKT4E-FITC and Leu4-PE energy transfer efficiency was down even further to 8% and barely detectable between OKT4-FITC to Leu-5b-PE.

### EXAMPLE 6

The VH and VL antibody chains were cloned from several isolated cells as described in example 5A-C. Sequencing was performed by SequiServe—Dr. Willi Metzger, Vaterstetten, Germany._For each single cell one VH and one VL sequence was selected, which displayed the complete sequence from the putative signal peptide cleavage site to the beginning of the constant regions including all functional sequence, the CDRs 1-3 and corresponding framework, which had no stop-codon mutation, nor frame shift and were clearly germ line sequences (except for scarce, presumably PCR derived, mutations). Nucleotide sequences of VH and VL derived from cell S2 are shown in FIG. 10 and in the sequence listing (SEQ ID NO.: 58 and 59 respectively). Nucleotide sequences of VH and VL derived from cell S9 are shown in FIG. 11 and in the sequence listing (SEQ ID NO.: 60 and 61 respectively). The generation of scFvs from isolated VH and VL sequences was performed as described in example 5D. Protein and nucleotide sequences of scFv VL-VH derived from cell S2 are shown in FIG. 12 and in the sequence listing (SEQ ID NO.: 62 and 63 respectively). The complementary determining regions (CDRs) comprised within the scFv derived from cell S2 as shown in FIG. 12 are listed in the sequence listing (SEQ ID NO.: 64 to 75). Protein and nucleotide sequences of scFv VL-VH derived from cell S9 are shown in FIG. 13 and in the sequence listing (SEQ ID NO.: 76 and 77 respectively). The complementary determining regions (CDRs) comprised within the scFv derived from cell S9 as shown in FIG. 13 are listed in the sequence listing (SEQ ID NO.: 78 to 89).

The isolated and verified VH and VL sequences could further be used to generate a variety of antibody constructs comprising single chain antibodies, bispecific antibody constructs and complete immunoglobulin formats.

### EXAMPLE 7

Expression and Purification of Cloned scFvs

Transfected CHO cells were grown in roller bottles with HyQ PF CHO modified DMEM medium (HyClone Corp., Logan, Utah, USA) for 7 days. At harvest cells were removed by centrifugation and the supernatant, containing the expressed protein, was stored at −20° C. The anti CD28 scFv proteins were isolated in two chromatographic purification steps. Herefore, Äkta FPLC System (Pharmacia, Tennenlohe, Germany) and Unicom Software were used for chromatography. All chemicals were of research grade and purchased from Sigma (Deisenhofen, Germany) or Merck (Darmstadt, Germany).

In a first step cation exchange chromatography (FIG. 14) was performed, using a XK 16/10 column (Amersham Biosciences Europe GmbH, Freiburg, Germany) that was loaded with Q Sepharose according to the manufacturers protocol. The column was equilibrated with buffer A (20 mM tris pH 7.5,) the cell culture supernatant (50 ml) was diluted 1:3 with buffer A and was applied to the column (10 ml) with a flow rate of 3 ml/min. After washing with buffer A, bound protein was eluted using a linear 0-50% gradient of buffer B (20 mM Tris pH 7.5, 1 M NaCl), followed by a step of 100% buffer B. The scFv protein eluted from the linear gradient at approx. 110 ml (FIG. 14) and was analyzed on SDS-Page and Western Blot (FIG. 16) for product content and was used for further purification.

In a second step gelfiltration chromatography (FIG. 15) was performed on a 16/60 HiPrep column with Superdex 200 (Amersham Biosciences Europe GmbH, Freiburg, Germany) equilibrated with PBS (Gibco Invitrogen Corp., Carlsbad, USA). Eluted protein samples (flow rate 1 ml/min) were subjected to SDS-Page and Western Blot for detection of the product (FIG. 17). The column was previously calibrated for molecular weight determination (molecular weight marker kit, Sigma MW GF-200, Sigma-Aldrich Chemie GmbH Munich, Germany).

The anti-CD28 scFv had a molecular weight of approx. 27 kD under native conditions as determined by size exclusion chromatography in PBS. The purity of all isolated scFv proteins was >95% as determined by SDS-PAGE (FIG. 17). SDS PAGE was performed under reducing conditions with precast 4-12% Bis Tris gels (Invitrogen GmbH, Karlsruhe, Germany) according to the manufacturers protocol. The molecular weight was determined with MultiMark protein standard (Invitrogen GmbH, Karlsruhe, Germany). The gel was stained with colloidal Coomassie according to Invitrogen protocol.

The anti-CD28 scFv protein was specifically detected by Western Blot (FIG. 16 and 17) at a molecular weight of 27 kD. Western Blot was performed with a Biotrace NT membrane (Pall Gelman GmbH, Dreieich, Germany) and the Invitrogen Blot Module according to the manufacturers protocol. The antibodies used were Penta His (Qiagen, Hilden, Germany) and goat-anti-mouse labeled with alkaline phosphatase (Sigma-Aldrich Chemie GmbH Munich, Germany), the staining solution was BCIP/NBT liquid (Sigma-Aldrich Chemie GmbH Munich, Germany).

Protein concentrations were determined using protein assay dye (MicroBCA, Pierce Biotechnology, Rockford, Ill., USA) and IgG (Bio-Rad Laboratories Inc., Hempstead, UK) as standard protein.

### EXAMPLE 8

Binding Assays To confirm the specificity of the scFv clones the purified scFv constructs were used in an ELISA binding assay.

Each well of a Maxisorp surface immunoplate (NUNC, Kamstrupvej, Denmark) was coated at 4° C. over night with 100 μl of a 10 μg/ml anti-Flag M2 antibody (Sigma-Aldrich Chemie GmbH Munich, Germany). The remaining free binding sites were blocked using 1% bovine serum albumin (Sigma-Aldrich Chemie GmbH Munich, Germany). Subsequently, wells were washed using 0.05% Tween in PBS. For measuring the binding affinity of the purified anti-CD28 scFvs of previous example 7 dilution series were performed in a range from 20.5 μg/ml to 0.16 μg/ml (FIG. 14A and B) and 101 μg/ml to 0.78 μg/ml (FIG. 14C and D). 1% BSA in PBS (Sigma-Aldrich Chemie GmbH Munich, Germany) served as control. Wells were washed again using 0.05% Tween in PBS. For the detection of the specific binding activity of the scFvs 100 μl of a 1 μg/ml recombinant fusion protein of human CD28-murine Ig conjugated to biotin (Ancell Corp., Bayport, USA; for description of the recombinant fusion protein see example 4B) in PBS with 1% BSA was added to each well and incubated for 1 hr at room temperature. As control biotin-conjugated recombinant human CD40-murine Ig (Ancell Corp., Bayport, USA) was used in parallel under the same conditions for the detection of murine Ig-specific binding.

After washing with 0.05% Tween in PBS. 100 μl of a 1:500 diluted streptavidin horse radish peroxidase-conjugate (Jackson Immuno Research Laboratories Inc., West Grove, Pa., USA) were added for 1 hr at room temperature. After washing, 100 μl of the substrate ABTS (Sigma-Aldrich Chemie GmbH Munich, Germany) was added to each well for 30 minutes at room temperature. The binding activity of the scFvs was measured at 405 nm using a PowerWaveX microplate spectrophotometer (Bio-Tek Instruments Inc., Winooski, Vt., USA).

The results of the ELISA assay shown in FIG. 18A and FIG. 18B demonstrate binding of the scFv antibody derived from cell S2 to the recombinant human CD28-murine Ig and to the recombinant human CD40-murine Ig antigens as compared to the scFv antibody derived from cell S3. The selection process was therefore successful in the case of the cell S2 although the binding of the antibody was directed to the Fc part of murine Ig, which is part of both recombinant fusion proteins (recombinant human CD28-murine Ig and recombinant human CD40-murine Ig).

The results of the ELISA assay shown in FIG. 18C and FIG. 18D demonstrate binding of the scFv antibody derived from cell S9 to the recombinant human CD28-murine Ig antigen as compared to the scFv antibody derived from cell S4. No binding was observed to recombinant human CD40-murine Ig antigen. The FRET-based selection process was therefore also successful in the case of the cell S9. The selected antibody was specific for the CD28 part of the antigen and not for the mulg part.

### EXAMPLE 9

FRET Versus “Multicolor” FACS

The goal of this experiment was to compare the method of the present invention (FRET) with a conventional multicolor FACS experiment. For this purpose human B cells were isolated from 250 ml heparinized blood of a healthy donor (for procedure see example 4A) and anti-CD28 positive B cells were determined by both methods.

A) Fluorescence Resonance Energy Transfer (FRET) Staining

For the FRET staining six labeling reactions were prepared. The first labeling reaction contained 400 000 unstained cells resuspended in a volume of 400 μl in 10% FCS/PBS. These cells were used for the calibration of the FACS machine. A second reaction contained 400 000 cells and was labeled with 2.5 μg/ml polyclonal goat anti-human IgD-fluorescein conjugate. This reaction was used to determine the proportion of naïve B cells within the cell population and for FL2-FL1 compensation. The third reaction contained 400 000 cells and was labeled with 2.5 μg/ml anti-CD19 phycoerythrin (PE) conjugate (Pharmingen/Becton Dickinson, Franklin Lakes, N.J., USA) to determine the purity of the B cells and to compensate FL1-FL2 and FL3-FL2. A fourth reaction contained 400 000 cells labeled with 2.5 μg/ml polyclonal goat anti-human IgD-fluorescein conjugate and 2.5 μg/ml anti-IgD-Alexa 546 conjugate. These cells were used as a positive control and for the selection of appropriate FRET signal settings (gating). A fifth reaction contained also 400 000 cells and was labeled with 2.5 μg/ml polyclonal goat anti-human IgD-Fluorescein conjugate and 2.5 μg/ml anti-CD19 PE conjugate. Those cells were used to determine a B cell specific quadrant containing CD19/IgD expressing naive B cells. The sixth reaction contained 5×10⁶ cells in a volume of 1 ml 10% FCS in PBS and was labeled with 2.5 μg/ml polyclonal goat anti-human IgD-fluorescein conjugate and 2.5 μg/ml rCD28-Alexa 546 conjugate. Fluorescein served as donor dye and Alexa 546 as acceptor dye. This FRET labeling reaction was used to identify recCD28-mulg-specific B cells from within the isolated B cell population. All described labeling reactions were incubated for 30 minutes at 4° C., then washed twice in FACS buffer (1% FCS, 0.05% sodium azid), and finally resuspended in 200 μl FACS buffer (reactions 1-5) or 1 ml FACS buffer (reaction 6). The reactions were stored at 4° C. in the dark.

B) Multicolor Staining

For the multicolor staining another six labeling reactions were prepared reactions 7-12). Labeling reaction seven contained 400 000 cells resuspended in a volume of 400 μl in 10% FCS in PBS labeled with 2.5 μg/ml anti-human IgD-biotin conjugate (200 μl anti human IgD 1 mg/ml, Dako, Hamburg, Germany+10 μl biotin-LC-LC-NHS 1.5 mg/ml, Pierce, Perbio, Bonn, Germany, incubated for 1 hr at room temperature). After 30 min, cells were washed with FACS buffer and resuspended in 200 μl FACS buffer. Subsequently, further labeling with 10 μl streptavidin-APC (Pharmingen/Becton Dickinson, Franklin Lakes, N.J., USA) was performed according to manufacturer's instructions. Cells were incubated for 30 min and washed with FACS buffer. These APC labeled cells were used as control for FL4 signal resulting from streptavidin APC and for FL3-FL4 compensation. In reaction number eight 400 000 cells were incubated with 10 μl streptavidin-APC according to manufacturer's instructions for control of unspecific binding of streptavidin-APC. The ninth reaction contained 400 000 cells labeled with 4 μl anti-human cytokeratin mouse IgG1 A45 B/B3-LC-LC-Biotin (0.5 mg/ml, 200 μl monoclonal mouse anti cytokeratin IgG 1 mg/ml, R002A, Micromet AG, Munich, Germany+10 μl Biotin-LC-LC-NHS 1.5 mg/ml, Pierce, Perbio, Bonn, Germany, incubated for 1 hr at room temperature). This reaction was incubated for 30 min, washed with FACS buffer and resuspended in 200μl FACS buffer. Subsequently, 10 μl streptavidin-APC were added according to manufacturer's instructions, the reaction incubated for 30 min and washed with FACS buffer. Reaction number nine was used as a control for unspecific staining of biotinylated isotype control antibody. The tenth reaction contained 5×10⁶ cells in a volume of 1 ml and was labeled with 2.5 μg/ml anti-human IgD-biotin, incubated for 30 min, washed with FACS buffer, and resuspended in 1 ml FACS buffer. The following was added to this preincubated reaction: 20 μl of streptavidin-APC, 38 μl of recCD28-mulg-fluorEscein (0.065 mg/ml), 38 μl of recCD28-mulg-Alexa 647 (0.065 mg/ml), 40 μl of anti-human CD3 PE (Pharmingen/Becton Dickinson, Franklin Lakes, N.J., USA) according to manufacturer's instructions. The reaction was incubated for 30 min and washed with FACS buffer. Reaction number ten was used to quantify the number of recCD28-mulg-specific naïve B cells identifiable by multicolor staining. The eleventh reaction contained 5×10⁶ cells and was labeled with 38 μl of recCD28-mulg-fluorescein (0.065 mg/ml, 100 μl rec CD28 mulg 0.5 mg/ml, Ancell Corp., Bayport, USA+5 μl fluorescein-NHS 1.3 mg/ml Fluka, Riedel-de Haen, Sigma-Aldrich, Seelze, Germany, incubated for 1 hr at room temperature), 38 μl of recCD28-mulg-Alexa 647 (0.065 mg/ml), 40 μl of anti-human CD3 PE according to manufacturer's instructions, and 20 μl of streptavidin-APC. Reaction number eleven served as control for unspecific streptavidin APC binding. In a twelfth reaction 5×10⁶ cells were preincubated with 10 μl A45 B/B3-LC-LC-Biotin (0.5 mg/ml) for 30 min, washed with FACS buffer, resuspended in 2001 μl FACS buffer and labeled with 10 μl streptavidin-APC according to manufacturer's instructions, 38 μl of recCD28-mulg-fluorescein (0.065 mg/ml), 38 μl of recCD28-mulg-Alexa 647 (0.065 mg/ml, 100 μl rCD28 0.5 mg/ml Ancell Corp., Bayport, USA+5 μl Alexa 647 NHS 1.5 mg/ml Molecular Probes, Eugene, Oreg., USA, incubated for 1 hr at room temperature), 40 μl of anti-human CD3 PE for 30 min and washed with FACS buffer. Reaction number twelve served as isotype control of reaction ten. The reactions were stored at 4° C. in the dark.

C) FACS Results

The FACS measurements for multicolor staining and FRET staining were performed using a two laser FACS Calibur (Beckton Dickinson, Franklin Lakes, N.J., USA). The fluorochromes fluorescein and PE were excited by a first laser at 488 nm. The fluorochromes Alexa 647 and APC were excited by the second laser at 630 nm. For the FRET measurements only the first laser at 488 nm was used. The measurement was performed in the presence of propidium iodide (FL3). All FL3 negative cells (=living cells) were selected via gating. Measurement of FRET labeling reaction two containing anti-IgD-fluorescein conjugate (FL1) allowed selection of living IgD positive B cells (FL1 positive, FL3 negative) in a region R1. Since it is known that false positive cells outside the B cell population scatter broadly in the FSC/SSC R1 was additionally gated back into the FSC/SSC window, which resulted in a new region R3.

To identify the FL1 positive plus FL2 positive cell population during FRET measurement, only those cells were selected, which were contained in R1 and R3. The setting of the FRET region (R4) within these parameters was determined by measurement of FRET labeling reaction four, which contained IgD double labeled cells (anti-IgD-fluorescein plus anti-IgD Alexa 546).

When FRET labeling reaction six was measured to detect rCD28 specific naïve B cells, a total number of 1 845 945 cells was analysed. From this number only 17 cells were identified within the FRET region R4. This equals a proportion of 0.000929%.

The multicolor FACS was performed using both lasers. The measurement could not be performed in the presence of propidium iodide, since all four filters of the FACS were in use for the optimal selection. IgD positive cells were detected using anti-IgD-biotin/streptavidin-APC in FL4. To eliminate false positive cells possibly originating from unspecific streptavidin-APC binding reaction eleven was used as control for out-gating. As isotype control (reaction twelve) a biotinylated anti-human cytokeratin antibody (A45 B/B3) was used. The rCD28 specific naive B cells in the multicolor FACS were expected to be fluorescein (FL1, recCD28-mulg-fluorescein) and Alexa 647 (FL3, recCD28-mulg-Alexa647) positive as well as APC (FL4, anti-IgD-biotin/streptavidin-APC) positive, but PE (Fl3, anti-CD3 PE) negative. These cells were identified in the FACS by setting the appropriate gates during measurement of labeling reaction ten.

A number of 1 487 595 cells from reaction ten were analyzed, whereby 312 cells appeared positive, which corresponds to 0.0210%. FACS measurements with the isotype control reaction twelve resulted in 285 positive cells out of 1730700 isolated cells. This corresponds to 0.016%. The difference between these two percentages indicates the “real” number of positive cells: 0.0045%.

These results show that a lower percentage of positive cells was detected with the FRET method according to the invention compared to multicolor FACS. These cells detected by the FRET method represent a B-cell population with anti-CD28 specificity occurring at extremely low frequency in human blood. The higher percentage of positive cells detected with multicolor FACS as shown in FIG. 19 is due to several technical features of this method. Unspecific staining and binding contributes to false positive results (U.S. Pat. No. 5,326,696). In contrast to multicolor FACS only the donor is excited by a specific wavelength in the FRET method leading to a low background noise and high sensitivity and specificity of this method. As already shown in Reference Example 1 human B cells with anti-EpCAM specificity were not detected by multicolor FACS. For those reasons the lower percentage of anti-CD28 positive B cells isolated by the FRET method relates to the higher specificity and sensitivity of this method compared to multicolor FACS. This higher selectivity amplifies to a considerable reduction of further screening efforts. Furthermore, the complexity of the experiment and the FACS device used was advantageously simplified with the FRET method in comparison to a conventional multi color FACS.

1. A method of identifying a B cell carrying a surface immunoglobulin molecule having a binding site for an antigen of interest comprising
(a) contacting a sample putatively containing said B cell
(aa) with the antigen of interest wherein said antigen is labeled with a first label; and
(ab) with a receptor specifically binding to said surface immunoglobulin molecule wherein said receptor is labeled with a second label; and wherein said first label, when being brought into a spatial proximity of between 10 and 100 Angstrom with said second label emits a detectable signal upon activation of said second label by an external source; and
(b) assessing the presence of said detectable signal, wherein said presence is, in turn, indicative of the B cell carrying a surface molecule having a binding site for the antigen of interest.

2. The method of claim 1, wherein said B cell is an autoreactive B cell.

3. The method of claim 1 wherein said surface immunoglobulin molecule is an IgD, an IgE, an IgM or an IgG.

4. The method of claim 1, wherein said B-cell is a naive, IgD-positive B-cell.

5. The method of claim 1, wherein said antigen of interest is selected from the group consisting of
(a) auto-antigens
(b) allergens; and
(c) immunoglobulins

6. The method of claim 1, wherein said sample is a sample of essentially purified B cells.

7. The method of claim 1, wherein said first label is a fluorophore or fluorochrome.

8. The method of claim 1, wherein said second label is a flurophore or fluorochrome.

9. The method of claim 1, wherein said receptor is an antibody or a fragment or derivative thereof.

10. The method of claim 9, wherein said antibody is directed against the Fc-part of the surface immunoglobulin molecule.

11. The method of claim 10, wherein said antibody is an anti-idiotypic antibody, wherein said anti-idiotypic antibody does not interfere with the binding site to the antigen.

12. The method of claim 1, further comprising the step of isolating identified B-cells.

13. The method of claim 12, further comprising the step of cloning VH- and VL-domains from identified B cells.

14. The method of claim 13, further comprising the steps of
(a) introducing mutations in the sequences encoding said VH- and/or VL-domain/s of at least one of said B cells, wherein said mutations result in amino acid substitutions and wherein the number of mutations ranges from one to thirty, such that one or more modified antibodies is/are obtained; and/or
(b) shuffling a repertoire of V-domains to the VH- or VL-domains of said B cells, such that one or more modified antibodies is/are obtained; and/or
(c) grafting at least one CDR of at least one of the cloned VH- and/or VL-domain/s of said B cells into the corresponding position/s of the variable regions of a first antibody library, such that a second antibody library is obtained; and
(d) subjecting the resulting modified antibody/ies and/or antibody library to further selection on the antigen or parts thereof using a biological display system.

15. The method of claim 13, further comprising the step of expressing at least one of said V-domains in an expression system.

16. The method of claim 13, further comprising the step of generating antibodies or fragments or derivatives from said V-domains.

17. The method of claim 16, further comprising the steps of rearranging possible combinations of different VH and VL domains.

18. The method of claim 13, wherein the VH and VL domains are specific for CD28.

19. The method of claim 18, wherein the VH- and/or VL-domains
(a) comprise an amino acid sequence selected from the group consisting of SEQ ID Nos: 78, 80, 82, 84, 86 and 88; or
(b) are encoded by a nucleic acid sequence comprising a sequence selected from the group consisting of SEQ ID NOs: 60, 61, 79, 81, 83, 85, 87 and 89.

20. The method of claim 16, wherein said derivatives are bispecific antibody constructs or single chain antibodies.

21. The method of claim 16, wherein said derivatives comprise at least one binding site specific for CD28.

22. The method of claim 21, wherein said derivatives
(a) comprise the amino acid sequence as set forth in SEQ ID NO: 76; or
(b) are encoded by a nucleic acid sequence comprising the sequence as set forth in SEQ ID NO: 77.

23. The method of claim 1, further comprising an assay for antibody evaluation.

24. The method of claim 23, wherein said evaluation assay is a binding assay.

25. The method of claim 24, wherein said binding assay is an ELISA or a FACS based binding assay.

26. An antibody generated by the method of claim 1, which is specific for human CD28.

27. An antibody generated by the method of claim 1, wherein said antibody
(a) comprises an amino acid sequence selected from the group consisting of SEQ ID NOs: 76, 78, 80, 82, 84, 86 and 88; or
b) is encoded by a nucleic acid sequence comprising a sequence selected from the group consisting of the SEQ ID NOs: 60, 61, 77, 79, 81, 83, 85, 87 and 89.

28. A device for assessing the presence of a detectable signal as defined in claim 1, wherein said device comprises a closed system for the detection laser-beam and a catcher tube, and wherein the B cell of interest can be collected as a single cell by means of an electrochemical device which is triggered by an electric signal generated by the FACS device, wherein the electrochemical device moves the nozzle of the steady catcher tube liquid stream for a programmed time over a collecting tube, microtiter plate or other container after a B cell is sorted.


SEQUENCE LISTING (89 sequences)

SEQ ID NO: 1 - DNA, 21 bp, artificial sequence
  mus musculus primer
accttcaacaccccagccatg

SEQ ID NO: 2 - DNA, 23 bp, artificial sequence
  mus musculus primer
gctcggtcaggatcttcatgagg

SEQ ID NO: 3 - DNA, 20 bp, artificial sequence
  mus musculus primer
gctacacattcagtagcttc

SEQ ID NO: 4 - DNA, 20 bp, artificial sequence
  mus musculus primer
gtatggcatgtttaccatcg

SEQ ID NO: 5 - DNA, 20 bp, artificial sequence
  mus musculus primer
tcagtagcttctggatagag

SEQ ID NO: 6 - DNA, 26 bp, artificial sequence
  mus musculus primer
gtatggcatgtttaccatcgtattac

SEQ ID NO: 7 - DNA, 20 bp, artificial sequence
  mus musculus primer
gttacaatttctccgacaag

SEQ ID NO: 8 - DNA, 20 bp, artificial sequence
  mus musculus primer
gtcgcaggcggaataatcac

SEQ ID NO: 9 - DNA, 20 bp, artificial sequence
  mus musculus primer
tctccgacaagtggattcac

SEQ ID NO: 10 - DNA, 20 bp, artificial sequence
  mus musculus primer
gcaggcggaataatcacccg

SEQ ID NO: 11 - DNA, 21 bp, artificial sequence
  homo sapiens primer
tggcagatgagcttggacttg

SEQ ID NO: 12 - DNA, 21 bp, artificial sequence
  homo sapiens primer
acactctcccctgttgaagct

SEQ ID NO: 13 - DNA, 20 bp, artificial sequence
  homo sapiens primer
gtgctcccttcatgcgtgac

SEQ ID NO: 14 - DNA, 21 bp, artificial sequence
  homo sapiens primer
actcgtcatactcctgcttgc

SEQ ID NO: 15 - DNA, 24 bp, artificial sequence
  homo sapiens primer
tggaagaggcacgttcttttcttt

SEQ ID NO: 16 - DNA, 20 bp, artificial sequence
  homo sapiens primer
agttacccgattggagggcg

SEQ ID NO: 17 - DNA, 19 bp, artificial sequence
  homo sapiens primer
ccttccaggccactgtcac

SEQ ID NO: 18 - DNA, 20 bp, artificial sequence
  homo sapiens primer
gtggggcgccccaggcacca

SEQ ID NO: 19 - DNA, 23 bp, artificial sequence
  homo sapiens primer
gatggaggcggcgatccacacgg

SEQ ID NO: 20 - DNA, 23 bp, artificial sequence
  homo sapiens primer
cagrtgcagctggtgcartctgg

SEQ ID NO: 21 - DNA, 23 bp, artificial sequence
  homo sapiens primer
saggtccagctggtrcagtctgg

SEQ ID NO: 22 - DNA, 23 bp, artificial sequence
  homo sapiens primer
caggtccagcttgtacagtctgg

SEQ ID NO: 23 - DNA, 23 bp, artificial sequence
  homo sapiens primer
sagrtcaccttgaaggagtctgg

SEQ ID NO: 24 - DNA, 23 bp, artificial sequence
  homo sapiens primer
saggtgcagctggtggartctgg

SEQ ID NO: 25 - DNA, 23 bp, artificial sequence
  homo sapiens primer
gaggtgcagctgktggagwcygg

SEQ ID NO: 26 - DNA, 23 bp, artificial sequence
  homo sapiens primer
cagctgcagctacagcagtgggg

SEQ ID NO: 27 - DNA, 23 bp, artificial sequence
  homo sapiens primer
cagstgcagctgcaggagtcsgg

SEQ ID NO: 28 - DNA, 23 bp, artificial sequence
  homo sapiens primer
gargtgcagctggtgcagtctgg

SEQ ID NO: 29 - DNA, 23 bp, artificial sequence
  homo sapiens primer
caggtacagctgcagcagtcagg

SEQ ID NO: 30 - DNA, 23 bp, artificial sequence
  homo sapiens primer
gacatccrgdtgacccagtctcc

SEQ ID NO: 31 - DNA, 23 bp, artificial sequence
  homo sapiens primer
gaaattgtrwtgacrcagtctcc

SEQ ID NO: 32 - DNA, 23 bp, artificial sequence
  homo sapiens primer
gatattgtgmtgacbcagwctcc

SEQ ID NO: 33 - DNA, 23 bp, artificial sequence
  homo sapiens primer
gaaacgacactcacgcagtctcc

SEQ ID NO: 34 - DNA, 23 bp, artificial sequence
  homo sapiens primer
gatgttgtgatgactcagtctcc

SEQ ID NO: 35 - DNA, 23 bp, artificial sequence
  homo sapiens primer
gatattgtgatgacccacactcc

SEQ ID NO: 36 - DNA, 23 bp, artificial sequence
  homo sapiens primer
gaaattgtgctgactcagtctcc

SEQ ID NO: 37 - DNA, 23 bp, artificial sequence
  homo sapiens primer
cagtctgtsbtgacgcagccgcc

SEQ ID NO: 38 - DNA, 22 bp, artificial sequence
  homo sapiens primer
tcctatgwgctgacwcagccac

SEQ ID NO: 39 - DNA, 23 bp, artificial sequence
  homo sapiens primer
tcctatgagctgayrcagcyacc

SEQ ID NO: 40 - DNA, 20 bp, artificial sequence
  homo sapiens primer
cagcctgtgctgactcaryc

SEQ ID NO: 41 - DNA, 23 bp, artificial sequence
  homo sapiens primer
cagdctgtggtgacycaggagcc

SEQ ID NO: 42 - DNA, 23 bp, Artificial Sequence
  homo sapiens primer
cagccwgkgctgactcagccmcc

SEQ ID NO: 43 - DNA, 23 bp, artificial sequence
  homo sapiens primer
tcctctgagctgastcaggascc

SEQ ID NO: 44 - DNA, 21 bp, artificial sequence
  homo sapiens primer
cagtctgyyctgaytcagcct

SEQ ID NO: 45 - DNA, 22 bp, artificial sequence
  homo sapiens primer
aattttatgctgactcagcccc

SEQ ID NO: 46 - DNA, 23 bp, artificial sequence
  homo sapiens primer
cagtctgtgctgactcagccacc

SEQ ID NO: 47 - DNA, 21 bp, artificial sequence
  homo sapiens primer
caatctgccctgactcagcct

SEQ ID NO: 48 - DNA, 23 bp, artificial sequence
  homo sapiens primer
tcttctgagctgactcaggaccc

SEQ ID NO: 49 - DNA, 23 bp, artificial sequence
  homo sapiens primer
cacgttatactgactcaaccgcc

SEQ ID NO: 50 - DNA, 23 bp, artificial sequence
  homo sapiens primer
caggctgtgctgactcagccgtc

SEQ ID NO: 51 - DNA, 23 bp, artificial sequence
  homo sapiens primer
cwgcctgtgctgactcagccmcc

SEQ ID NO: 52 - DNA, 20 bp, artificial sequence
  homo sapiens primer
ggttggggcggatgcactcc

SEQ ID NO: 53 - DNA, 20 bp, artificial sequence
  homo sapiens primer
gatggtgcagccacagttcg

SEQ ID NO: 54 - DNA, 21 bp, artificial sequence
  homo sapiens primer
ggagggygggaacagagtgac

SEQ ID NO: 55 - DNA, 24 bp, artificial sequence
  homo sapiens primer
ctccttaatgtcacgcacgatttc

SEQ ID NO: 56 - DNA, 31 bp, artificial sequence
  homo sapiens primer
  misc_feature (31)..(31): n = a, c, g, or t/u
ggagccgccgccgccagaaccaccaccaccn

SEQ ID NO: 57 - DNA, 34 bp, artificial sequence
  homo sapiens primer
  misc_feature (34)..(34): n denotes a variable number of nucleotides which are part of the sequence of specific VH clones
tctggcggcggcggctccggtggtggtggttctn

SEQ ID NO: 58 - DNA, 372 bp, Homo sapiens
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggaagtgggagctactattactactactactactacatggacgtctggggcaaagggaccacggtcaccgtctcctca

SEQ ID NO: 59 - DNA, 339 bp, Homo sapiens
gacatccaggtgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagtattatagtaccccactcactttcggcggagggaccaaggtggagatcaaa

SEQ ID NO: 60 - DNA, 360 bp, Homo sapiens
gaggtgcagctggtggagtctggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagagggtggacctctcatcatgggtgcttttgatatctggggccaagggacaatggtcaccgtctcttca

SEQ ID NO: 61 - DNA, 324 bp, Homo sapiens
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccttttggtgttcggcggagggaccaagctgaccgtccta

SEQ ID NO: 62 - protein, 252 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIKGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGSYYYYYYYYMDVWGKGTTVTVSS

SEQ ID NO: 63 - DNA, 756 bp, Homo sapiens
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcagtattatagtaccccactcactttcggcggagggaccaaggtggagatcaaaggtggtggtggttctggcggcggcggctccggtggtggtggttctgaggtgcagctgctggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggaagtgggagctactattactactactactactacatggacgtctggggcaaagggaccacggtcaccgtctcctca

SEQ ID NO: 64 - protein, 17 aa, Homo sapiens
KSSQSVLYSSNNKNYLA

SEQ ID NO: 65 - DNA, 51 bp, Homo sapiens
aagtccagccagagtgttttatacagctccaacaataagaactacttagct

SEQ ID NO: 66 - protein, 7 aa, Homo sapiens
WASTRES

SEQ ID NO: 67 - DNA, 21 bp, Homo sapiens
tgggcatctacccgggaatcc

SEQ ID NO: 68 - protein, 9 aa, Homo sapiens
QQYYSTPLT

SEQ ID NO: 69 - DNA, 27 bp, Homo sapiens
cagcagtattatagtaccccactcact

SEQ ID NO: 70 - protein, 10 aa, Homo sapiens
GFTFSSYAMS

SEQ ID NO: 71 - DNA, 30 bp, Homo sapiens
ggattcacctttagcagctatgccatgagc

SEQ ID NO: 72 - protein, 17 aa, Homo sapiens
AISGSGGSTYYADSVKG

SEQ ID NO: 73 - DNA, 51 bp, Homo sapiens
gctattagtggtagtggtggtagcacatactacgcagactccgtgaagggc

SEQ ID NO: 74 - protein, 15 aa, Homo sapiens
GSGSYYYYYYYYMDV

SEQ ID NO: 75 - DNA, 45 bp, Homo sapiens
ggaagtgggagctactattactactactactactacatggacgtc

SEQ ID NO: 76 - protein, 243 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNLLVFGGGTKLTVLGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAREGGPLIMGAFDIWGQGTMVTVSS

SEQ ID NO: 77 - DNA, 729 bp, Homo sapiens
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccttttggtgttcggcggagggaccaagctgaccgtcctaggtggtggtggttctggcggcggcggctccggtggtggtggttctcaggtgcagctgcaggagtctggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagagggtggacctctcatcatgggtgcttttgatatctggggccaagggacaatggtcaccgtctcttca

SEQ ID NO: 78 - protein, 11 aa, Homo sapiens
QGDSLRSYYAS

SEQ ID NO: 79 - DNA, 33 bp, Homo sapiens
caaggagacagcctcagaagctattatgcaagc

SEQ ID NO: 80 - protein, 7 aa, Homo sapiens
GKNNRPS

SEQ ID NO: 81 - DNA, 21 bp, Homo sapiens
ggtaaaaacaaccggccctca

SEQ ID NO: 82 - protein, 11 aa, Homo sapiens
NSRDSSGNLLV

SEQ ID NO: 83 - DNA, 33 bp, Homo sapiens
aactcccgggacagcagtggtaaccttttggtg

SEQ ID NO: 84 - protein, 10 aa, Homo sapiens
GGSISSYYWS

SEQ ID NO: 85 - DNA, 30 bp, Homo sapiens
ggtggctccatcagtagttactactggagc

SEQ ID NO: 86 - protein, 16 aa, Homo sapiens
YIYYSGSTNYNPSLKS

SEQ ID NO: 87 - DNA, 48 bp, Homo sapiens
tatatctattacagtgggagcaccaactacaacccctccctcaagagt

SEQ ID NO: 88 - protein, 12 aa, Homo sapiens
EGGPLIMGAFDI

SEQ ID NO: 89 - DNA, 36 bp, Homo sapiens
gagggtggacctctcatcatgggtgcttttgatatc